(19) United States
(12) Patent Application Publication
(10) Pub. No.: US 2025/0299771 A1
(43) Pub. Date: Sep. 25, 2025

(54) SYSTEMS AND METHODS FOR GENERATING CHIMERIC MAJOR HISTOCOMPATIBILITY COMPLEX (MHC) MOLECULES WITH DESIRED PEPTIDE-BINDING SPECIFICITIES

(71) Applicant: The Children's Hospital of Philadelphia, Philadelphia, PA (US)

(72) Inventors: Nikolaos SGOURAKIS, Philadelpha, PA (US); Omar ANI, Philadelphia, PA (US)

(21) Appl. No.: 18/840,413

(22) PCT Filed: Feb. 21, 2023
(86) PCT No.: PCT/US2023/062915

§ 371 Date: Aug. 21, 2024

Related U.S. Application Data
(60) Provisional application No. 63/312,584, filed on Feb. 22, 2022.
(60) Provisional application No. 63/269,962, filed on Mar. 25, 2022.

(51) Int. Cl. G16B 15/30; A61K 40/11; A61K 40/42; C07K 14/74; C12N 5/0783; G01N 33/68; G16C 20/50

(52) U.S. Cl. CPC G16B 15/30; A61K 40/11; A61K 40/421; C07K 14/70539; C12N 5/0636; G01N 33/6848; G16C 20/50; C12N 2510/00; G01N 2333/70539; G01N 2570/00

(57) ABSTRACT

The present invention relates to engineering synthetic MHC molecules with novel peptide binding properties, by exploring combinations of groove specificities from naturally occurring MHC-I alleles using structure-guided modeling and design. The invention also relates to generating a chimera, each involving computer implementation, storage of data on a memory device, and the data including data set(s) for making comparisons and accepting or rejecting structures, and with each involving synthesis and expression, including as herein further discussed:

### RELATED APPLICATIONS AND INCORPORATION BY REFERENCE

This application is a National Stage of International Application No. PCT/US2023/062915, filed 21 Feb. 2023, which claims benefit of and priority to Ser. Nos. 63/269,962 filed 25 Mar. 2022 and 63/312,584 filed 22 Feb. 2022. Reference is also made to Ser. Nos. 63/373,932 filed 30 Aug. 2022, 63/368,069 filed 11 Jul. 2022, and 63/312,599 filed 22 Feb. 2022. Reference is made to US Patent Publication Nos. US 2021/0052695, US 2021/0155670, US 2021/0269503 and US 2021/0371498 A1; U.S. Pat. No. 10,816,543 and International Patent Publication Nos. WO 2019/145509, WO 2020/010261 and WO 2021/138688.

The foregoing patent publications and applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FEDERAL FUNDING LEGEND

This invention was made with government support under Grant Nos. AI143997 and GM125034 awarded by the National Institutes of Health. The government has certain rights in the invention.

### REFERENCE TO SEQUENCE LISTING SUBMITTED ELECTRONICALLY

This application contains a sequence listing, which is submitted electronically. The contents of the electronic sequence listing (074313.1US4 Sequence Listing.xml; Size 40,596 bytes; and Date of Creation: Mar. 26, 2025) is herein incorporated by reference in its entirety.

### FIELD OF THE INVENTION

The present invention relates to engineering synthetic major histocompatibility complex (MHC) molecules with novel peptide binding properties, by exploring combinations of groove specificities from naturally occurring MHC-I alleles using structure-guided modeling and design.

### BACKGROUND OF THE INVENTION

The immune system can respond to a plethora of continuously evolving intracellular threats, such as viruses, pathogenic bacteria, and cancerous cells. Immune surveillance at the cellular level is dependent on distinguishing self-proteins, which are expressed by the host's own genes and facilitate physiological cell function, from aberrantly expressed proteins, expressed by the virulent genes of infectious agents or by the host's mutated oncogenes. In jawed vertebrates, this surveillance process is made possible by a complex intracellular processing system, enabled by the proteins of the Major Histocompatibility Complex (MHC) (Blum et al., 2013).

Class I MHC (MHC-I) proteins are expressed in all nucleated cells, and they are implicated in aspects of most, if not all, adaptive immune responses. They function by detecting aberrantly expressed proteins and alerting the immune system to the presence of intracellular threats by interacting with specialized receptors on T cells and Natural Killer (NK) cells (Rossjohn et al., 2015; Thompson, 1995). In particular, the structure of MHC-I proteins contains a peptide binding groove which can capture short (8-15 amino acids, where 9-10 is the optimal length) peptide “barcodes” derived from all proteins that are synthesized inside the cell (Falk et al., 1991; Yewdell and Bennink, 1999). (See also FIG. 1 providing graphical depictions of binding properties as related to groove MHC, base MHC and chimera MHC.) As part of a homeostatic protein turnover mechanism called the endogenous antigen processing and presentation (APP) pathway, cellular proteins are eventually degraded by the proteasome, which breaks down linear amino acid sequences into shorter peptide fragments (FIG. 2) (Sijts and Kloetzel, 2011). Peptides of a specific length range (preferably from 8-16 amino acids) are then transported into the Endoplasmic Reticulum (ER) lumen through the Transporter Associated with antigen Processing (TAP), where they are further processed by aminopeptidases (ERAP1-2) (Saveanu et al., 2005), which trim the N-terminus of some peptides (Hammer et al., 2006), and then loaded onto nascent MHC-I molecules to create stable peptide-MHC (pMHC) protein complexes (Androlewicz, 1999). pMHC molecules egress to the cell surface, where they can be surveilled by immune cells, such as T cells and NK cells to drive self- vs non-self discrimination and adaptive immune responses (Rossjohn et al., 2015). Through this process, a pool of approximately 100,000 different peptide “barcodes” are continuously processed and displayed on the surface, as a means of signaling the cellular homeostatic state to the immune system. MHC-I proteins are therefore the cornerstone of immune monitoring, and de-regulation of the APP can lead to several diseased states, including immunodeficiencies (Zimmer et al., 2005), viral immune evasion and the establishment of latency (Koutsakos et al., 2019), autoimmunity (Riedhammer and Weissert, 2015), and cancer (Dhatchinamoorthy et al., 2021).

In humans, there are thousands of different allotypes of class I HLA (Human Leucocyte Antigen, the human MHC) proteins, encoded by the HLA locus found at the short arm of chromosome 6. Classical HLA genes are further classified in 3 sub-classes HLA-A, HLA-B and HLA-C (Vita et al., 2019). An individual's genotype therefore comprises 6 class-I HLA genes, which, given the highly polymorphic nature of the MHC-I peptide binding groove, can create an unlimited number of combinations at the population level, ensuring species adaptability to emerging pathogenic strains.

Despite being the most polymorphic proteins in the human genome, MHC-I (HLA) molecules share a conserved domain structure consisting of a polymorphic heavy chain, an invariant light chain or beta-2 microglobulin (β₂m), and bound peptide. The MHC-I heavy chain comprises immunoglobulin-like domains α₁, α₂ and α₃, where domains α₁ and α₂ define a peptide binding groove in the MHC-I 3D structure, while the α₃ domain stabilizes the molecule by creating an extensive binding interface with β₂m (FIG. 3). The peptide binding groove of MHC-I molecules is made up of adjacent “pockets” A-F, which accommodate the peptide in a mostly extended backbone conformation. Amino acid polymorphisms located along the peptide binding groove define a repertoire of 10⁴-10⁶ different peptide sequences which can be recognized by each MHC-I allotype, to ensure that a wide range of peptide “barcodes” can be sampled by the pathogen or cancer proteomes and displayed at the cell surface.

The repertoire of peptides which can bind to a given MHC-I allele is often represented in compact form as a sequence “logo”, where for each position P1 . . . P9 the relative frequency of different amino acid types is shown (FIG. 4) (Schneider and Stephens, 1990). In most HLA allotypes, such as HLA-A*02:01, a common allele across multiple ethnic groups, the primary stabilizing interactions between the peptide and MHC-I are contributed by pockets B and F, which anchor the 2^{nd} (P2) and 9^{th} (P9) residues of the peptide. The peptide binding groove of some HLA allotypes (e.g. HLA-B*08:01) forms strong, stabilizing interactions with additional peptide residues, and as a result the corresponding peptide sequence logos exhibit more than two conserved anchor positions (e.g. positions P3, P5 and P9 for HLA-B*08:01-FIG. 4) (Rasmussen et al., 2014; Smith et al., 2014). An in silico functional clustering and classification of 121 common HLA-A, -B and -C allotypes based on their corresponding peptide sequence logos was performed by Rassmusen et al. (FIG. 5) (Rasmussen et al., 2014). This analysis has shown a wide distribution of peptides which can be displayed by different HLA molecules including charged, polar or hydrophobic amino acids and their combinations at defined anchor positions, indicating that the MHC-I structure can accommodate a diverse set of peptide sequences. Notwithstanding, the peptide binding logos of known HLA alleles do not cover the entire range of 9mer peptide sequences, and as a result the displayed repertoire of peptides at the population level contains blind spots of “forbidden” peptides (Lee et al., 2021). In addition, there is a large pool of HLA sequences that have not yet been explored by evolutionary processes.

Citation or identification of any document in this application is not an admission that such document is available as prior art to the present invention.

### SUMMARY OF THE INVENTION

In MHC-I structures, the peptide binding groove comprises of approx. 37 residues which define a combinatorial space of up to 20³⁷(10⁴⁸) potential HLA sequences with distinct peptide binding properties. Many of these sequences may disrupt proper folding of the MHC-I structure to a global free energy minimum by introducing “frustration” at the folding energy landscape. However, a subset of such synthetic HLA sequences produce molecules with a properly conformed peptide binding groove of novel peptide binding specificities.

Exploring the space of HLA sequences using in silico protein design methods, followed by established protein refolding, stability measurements and peptide binding assays in vitro provide the means to test the origin of stability and peptide specificity of the MHC-I peptide binding groove, but can also have important biomedical ramifications. Expanding the peptide binding repertoire beyond that which is naturally sampled by classical HLA alleles through the design of synthetic molecules is relevant for several applications, including eliciting antitumor immunity by breaking T cell self-tolerance for specific peptide antigens (Parks et al., 2019), T cell vaccine development approaches to treat infections, autoimmunity and cancer, and the development of custom peptide binders for biosensing (summarized in FIG. 6).

As a first step towards this goal, Applicants outline a rational approach for engineering synthetic MHC molecules with novel peptide binding properties, by exploring combinations of groove specificities from naturally occurring MHC-I alleles (both human and non-human) using structure-guided modeling and design. Finally, Applicants provide proof-of-concept in vitro data for 3 synthetic MHC molecules which validate the approach.

The invention relates to a computer-assisted method for identifying or designing potential compounds to fit within or bind to an MHC chimera (“chimera”) or a functional portion thereof, or a computer-assisted method for identifying or designing a potential chimera or a functional portion thereof for binding to a desired compounds, or a computer-assisted method for identifying or designing a potential chimera of interest, optionally with regard to predicting area(s) of the chimera to be able to be manipulated, said method comprising using a computer system, optionally comprising one or more of a programmed computer comprising a processor, a data storage system, an input device, and an output device, and said method comprising steps comprising: (a) inputting into the programmed computer through said input device data comprising the three-dimensional co-ordinates of a subset of the atoms from or pertaining to MHC chimera crystal structure, thereby generating a data set; (b) comparing, using said processor, said data set to a computer database of structures stored in said computer data storage system, e.g., structures of compounds that bind or putatively bind or that are desired to bind to a chimera of the present invention or as to a chimera structure; (c) selecting from said database, using computer methods, structure(s), optionally comprising structure(s) of chimera(s) that may bind to desired structures, and/or desired structures that may bind to certain chimera(s) or portions thereof, and/or portions of the chimera(s) that may be manipulated; (d) constructing, using computer methods, a model of the selected structure(s); and (e) outputting to said output device the selected structure(s); and (f) optionally synthesizing one or more of the selected structure(s); and further (g) optionally testing said synthesized selected structure(s) as or in a chimera.

The invention relates to performing a combination of the steps summarized above according to a first embodiment for generation of chimera, a second embodiment for generation of chimera, or a third embodiment for generation of chimera.

The invention encompasses methods comprising storage of data on a memory device, and the data including learning data set(s) for making comparisons and accepting or rejecting structures.

In another embodiment, step (f), or steps (f) and (g) of the above method are performed.

In another embodiment, steps (f) or (f) and (g) include synthesis and expression, said expression optionally being via a vector, or in a cell, a mammalian cell, or a human cell, or a non-human primate cell, or a non-human mammal cell, or a bacterial cell or in E. coli.

In another embodiment, steps (f) or (f) and (g) include incubating the chimera with a sample containing a peptide of interest and optionally include binding of peptide to chimera promotes folding of the peptide/MHC/b2m protein complex.

In another embodiment, the method further comprises detecting folding via antibody-based analysis and optionally comprises ELISA and further optionally comprises contacting with antibody W6/32.

In another embodiment, the method further comprises purification and optionally comprises affinity-based purification, of pMHC proteins and elution of bound peptides resulting in a purified product.

In another embodiment, the method further comprises analysis of purified product, optionally comprising proteomics analysis and optionally comprises performing mass spectrometry).

In another embodiment, the method further comprises inputting data or results of performing steps into the data set(s) or the memory device or stored data thereon for being employed in further computer implementation of a herein disclosed method or any performance of any of the first embodiment for generation of chimera, or the second embodiment for generation of chimera, or third embodiment for generation of chimera.

In another embodiment, the method further comprises contacting T-cell(s) with the chimera to obtain modified T-cell(s) comprising T-cell(s) identified by recognition of a chimera peptide: MHC complex, and optionally further comprising expanding the T-cell(s) into a modified T-cell population, wherein the T-cell(s) used in the contacting can be isolated from a patient or subject, and optionally altered therefrom by having or introducing desired coding nucleic acid molecule(s) and/or by expressing desired product(s), optionally said introducing through a lentivirus system. The T cell(s) used in the contacting can having a particular TCR expressed, e.g., by genetic modification, or naturally, or a can be a CAR-T, e.g., a T cell altered to express certain antigen receptor(s), or the T-cell can be patient-derived such as a Tumor Infiltrating Lymphocyte. The chimera can be provided into the system as a selection marker, e.g., in a bead or tetramerized form.

The invention further relates to use of modified T-cells for, or a method for, antigen targeting, said use or method comprising contacting modified T-cell(s) or the modified T-cell population with a sample.

In another embodiment, the method comprises inputting data or results of performing steps of said claim(s) into the data set(s) or the memory device or stored data thereon for being employed in further computer implementation of a herein disclosed method or any performance of any of the first embodiment for generation of chimera, or the second embodiment for generation of chimera, or third embodiment for generation of chimera.

The invention also comprises genetically modifying a dendritic cell optionally comprising genetically modifying a dendritic cell via a CRISPR system optionally comprising a CRISPR-Cas9 system, whereby coding for the chimeric is inserted into the genome of the dendritic cell, whereby there is a genetically modified dendritic cell that contains DNA coding for and/or expresses the chimera; and optionally expanding the modified dendritic into a modified T-cell population that contains DNA coding for and/or expresses the chimera, whereby the modified T-cell can target an antigen of interest.

In one embodiment, the antigen of interest is on a cell (cell having the antigen of interest). In another embodiment, the cell having the antigen of interest is a cancer cell, optionally a solid tumor cell or cell of a solid cancer.

The invention also comprises use of modified T-cells for, or a method for, antigen targeting, said use or method comprising contacting modified T-cell(s) or a modified T-cell population, with a sample; optionally wherein the sample comprises a cell or a cancer cell or a solid tumor cell or a cell of a solid cancer.

In one embodiment, the use further comprises inputting data or results of performing steps of said claim(s) into the data set(s) or the memory device or stored data thereon for being employed in further computer implementation of a herein disclosed method or any performance of any of the first embodiment for generation of chimera, or the second embodiment for generation of chimera, or third embodiment for generation of chimera.

The invention further relates to a composition, optionally a pharmaceutical or veterinary composition, comprising a chimera or a dendritic cell or a T-cell or a population of T-cells and a diluent, carrier or excipient, optionally a pharmaceutically acceptable or veterinarily acceptable diluent, carrier or excipient.

The invention further encompasses a dendritic cell or a T-cell or a population of T-cells.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 illustrates engineering MHC chimeras to decompose peptide binding from receptor interactions. Top: The peptide (ELNRKMIYM; SEQ ID NO: 1) binds with high affinity to the Groove MHC (HLA-B*08:01), but does not bind to the base MHC (HLA-A*02:01). Applicants hypothesize that only a subset of the polymorphic sites between the groove MHC and the base MHC dictate peptide binding specificity. Mutating this subset of polymorphic positions in the base MHC sequence to match the corresponding groove MHC residues generates a chimeric MHC that has the surface structure of the Base MHC and binds the peptide presented by the Groove MHC. Bottom: The sequence logo of HLA-B*08:01 and HLA-A*02:01 from netMHCpan motif viewer, and the predicted sequence logo for the chimeric MHC which exactly matches the sequence logo of HLA-B*08:01 (Reynisson et al., 2020).

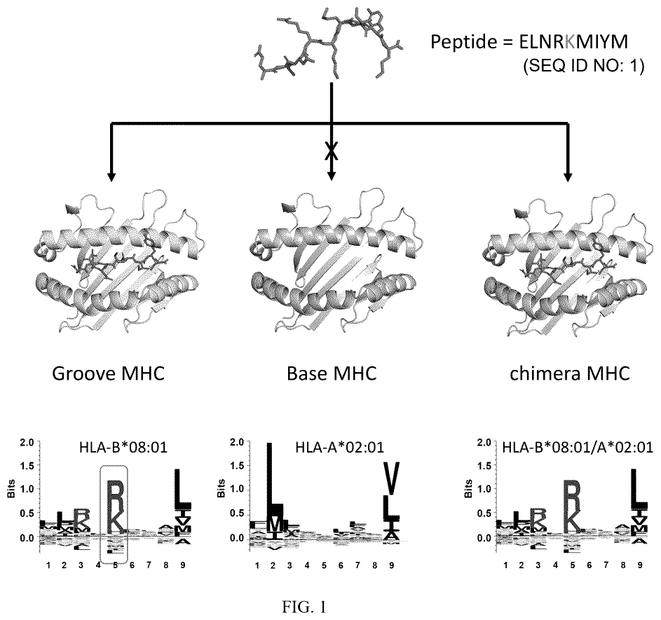

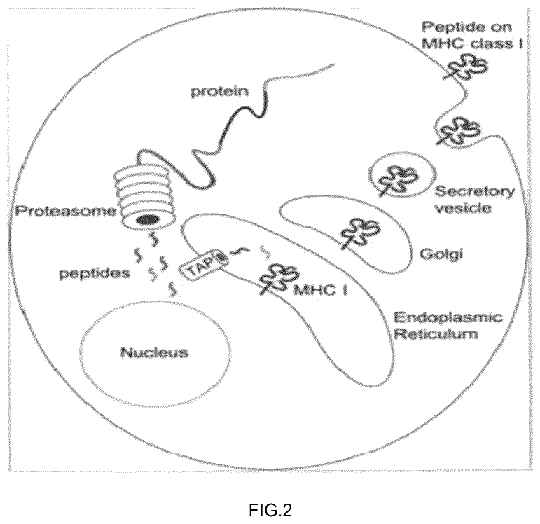

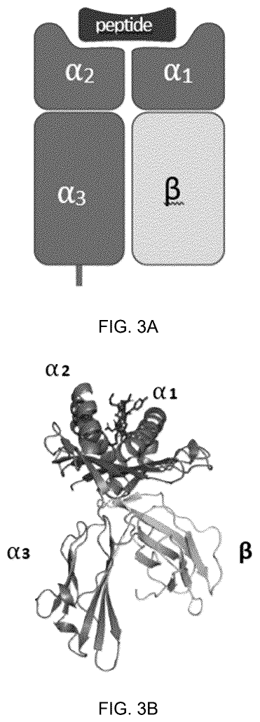

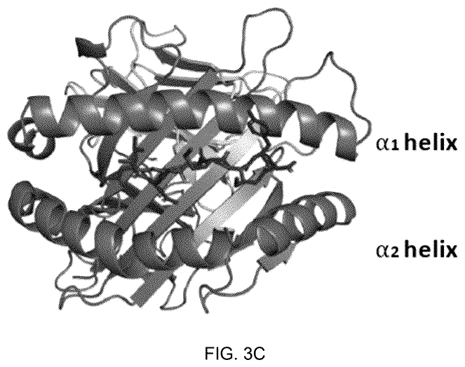

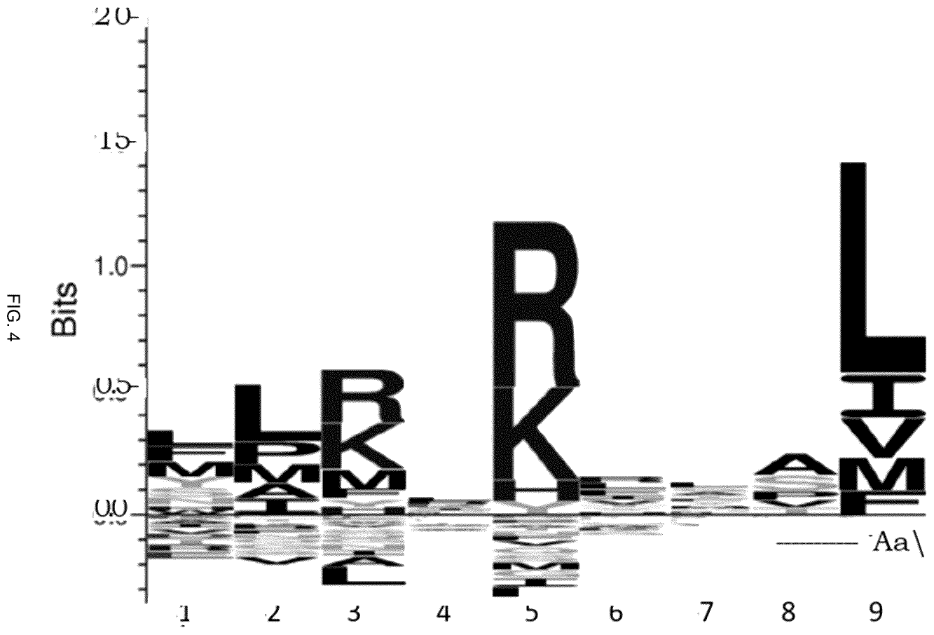

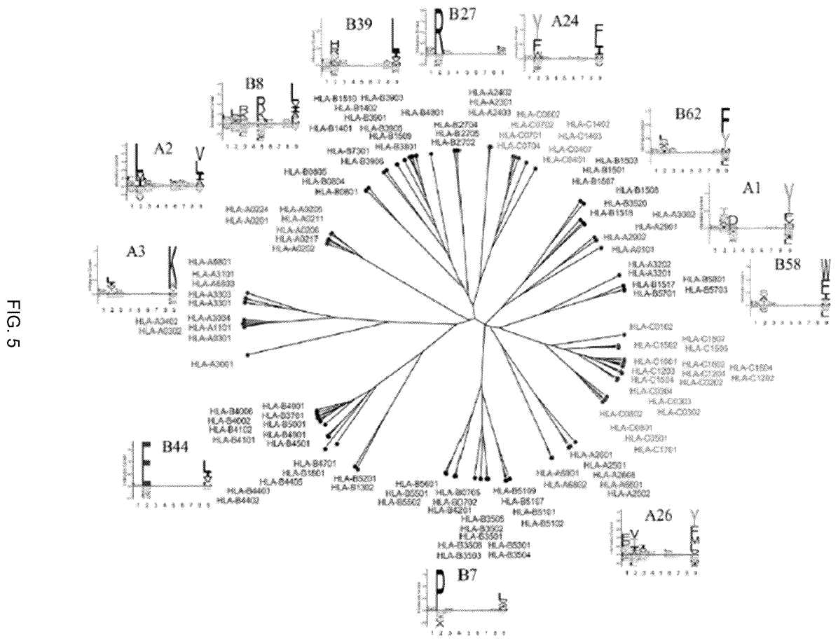

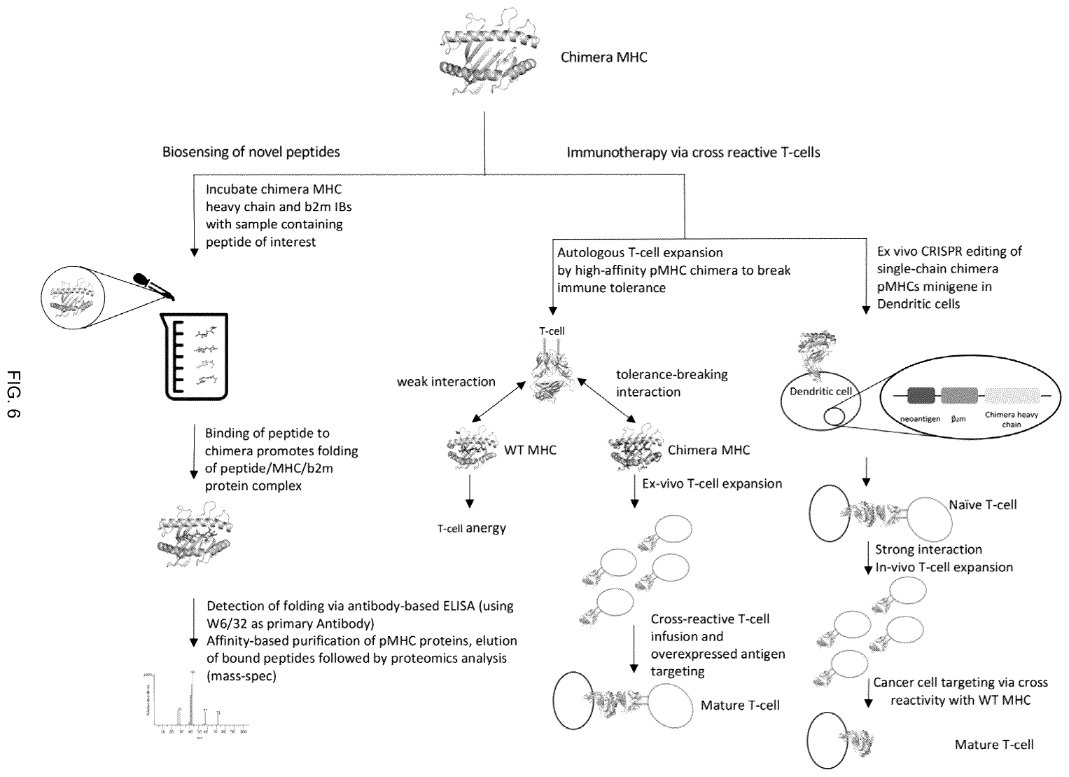

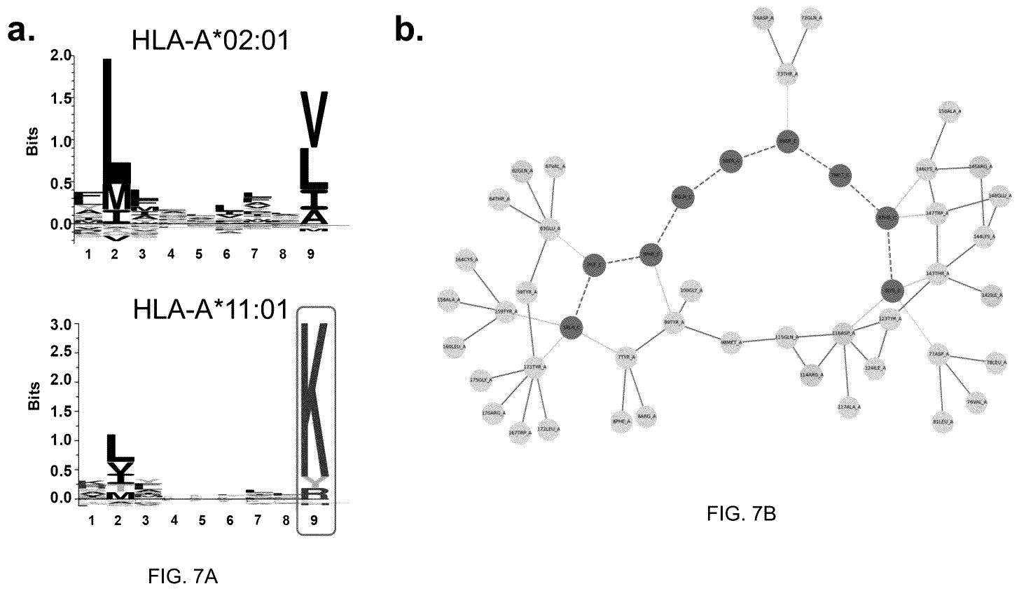

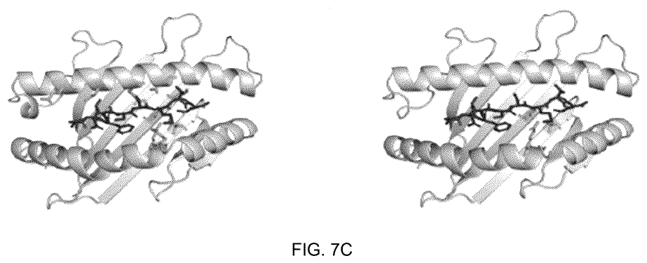

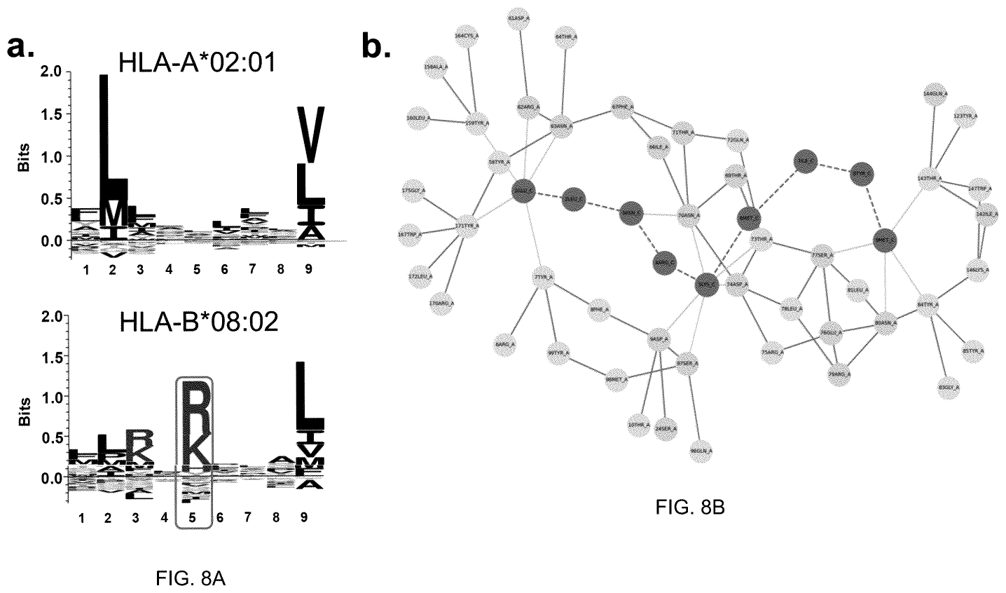

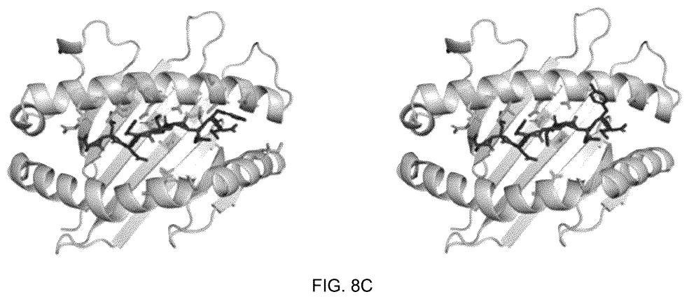

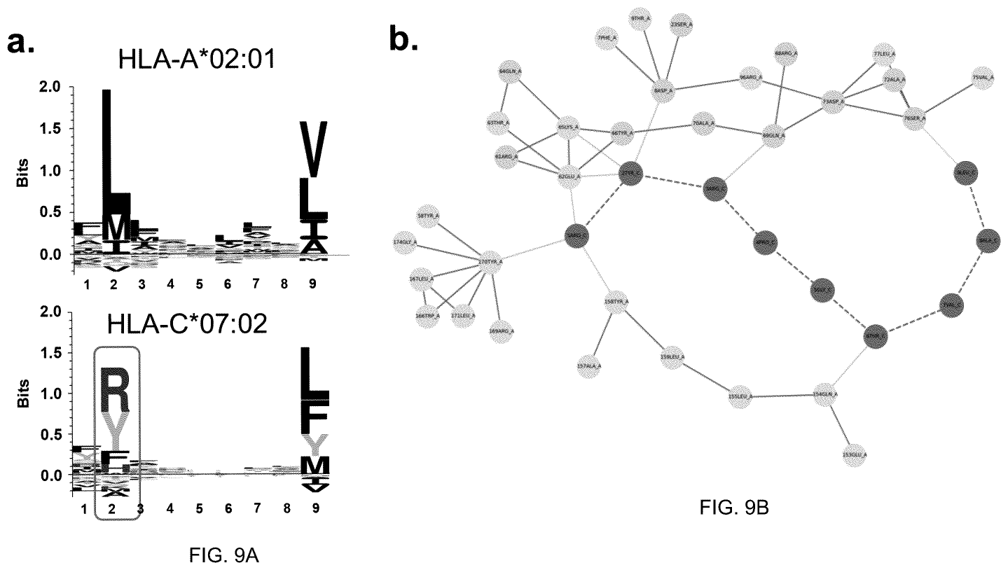

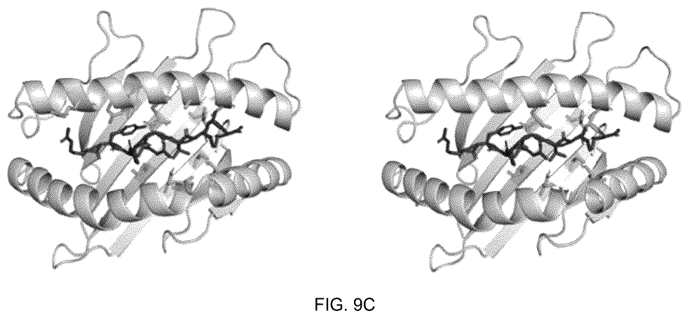

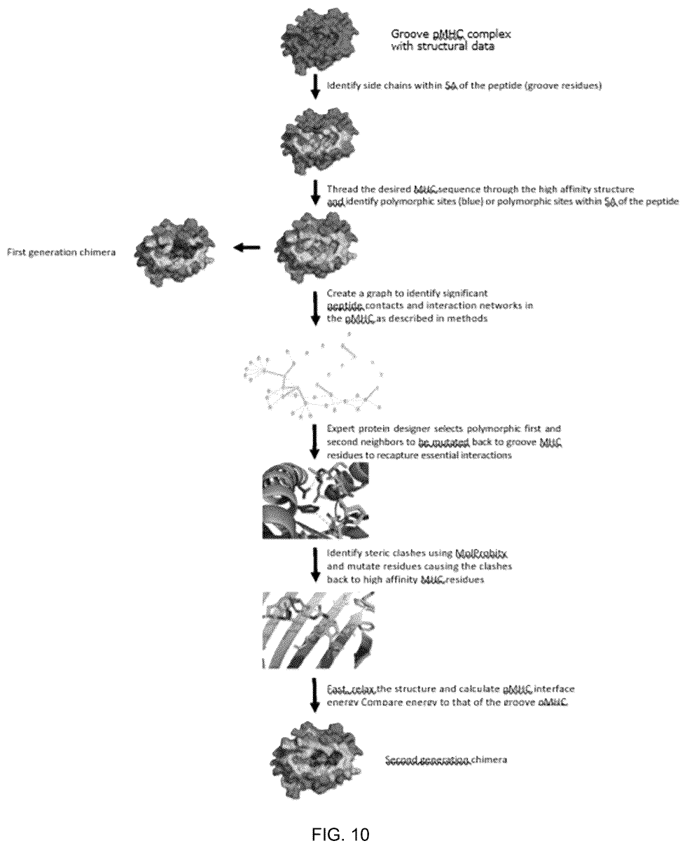

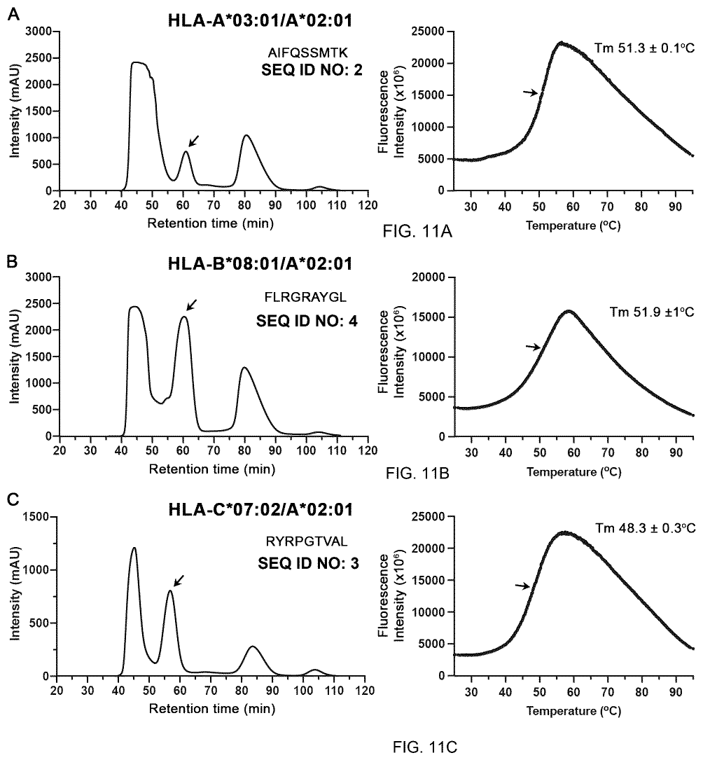

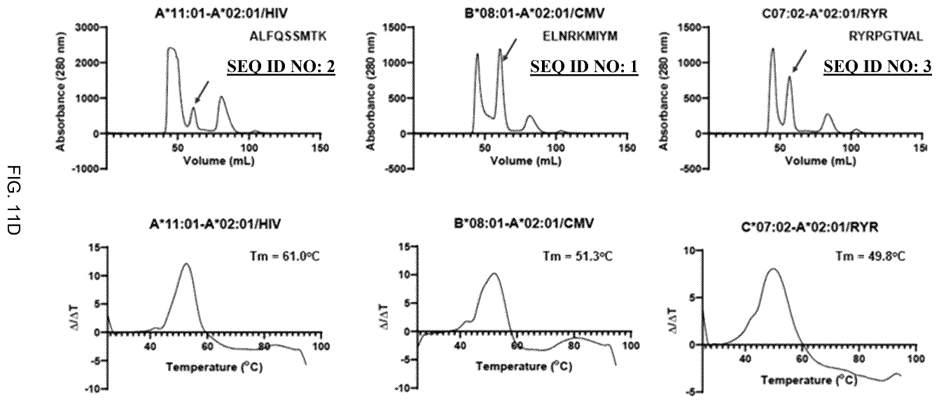

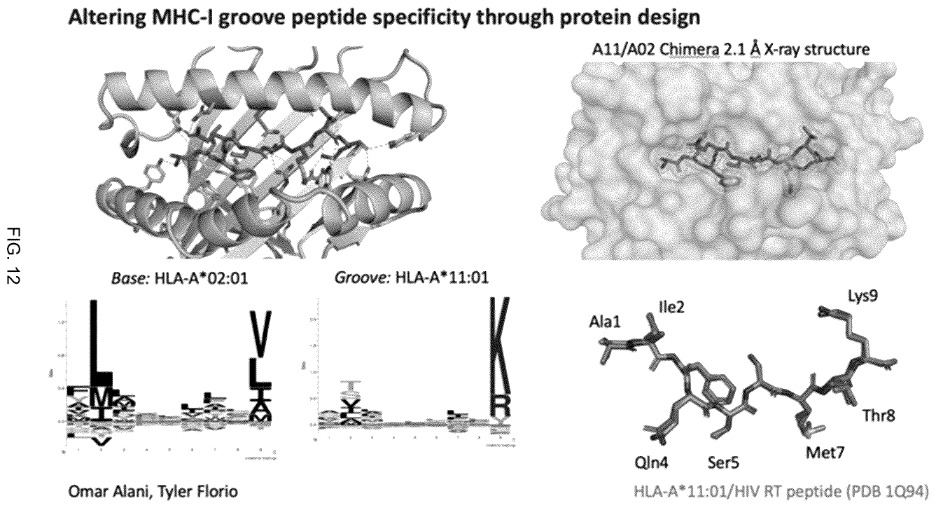

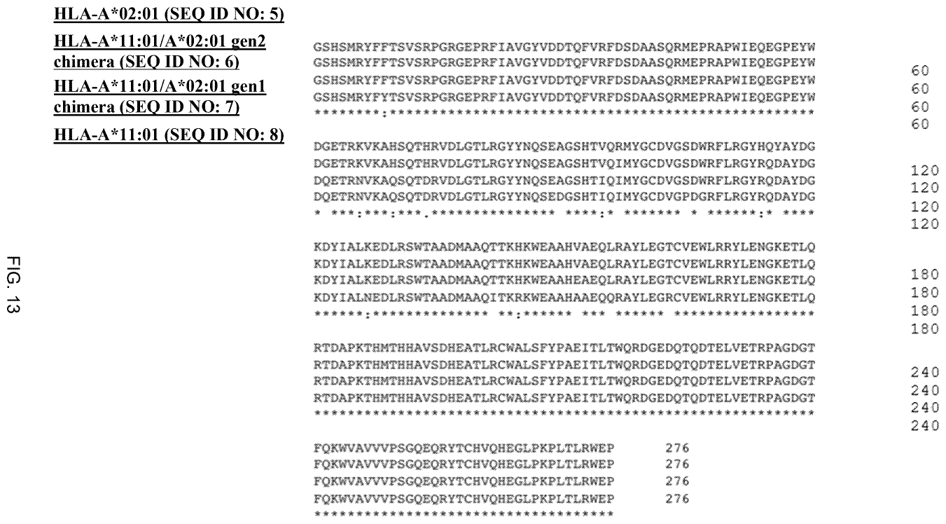

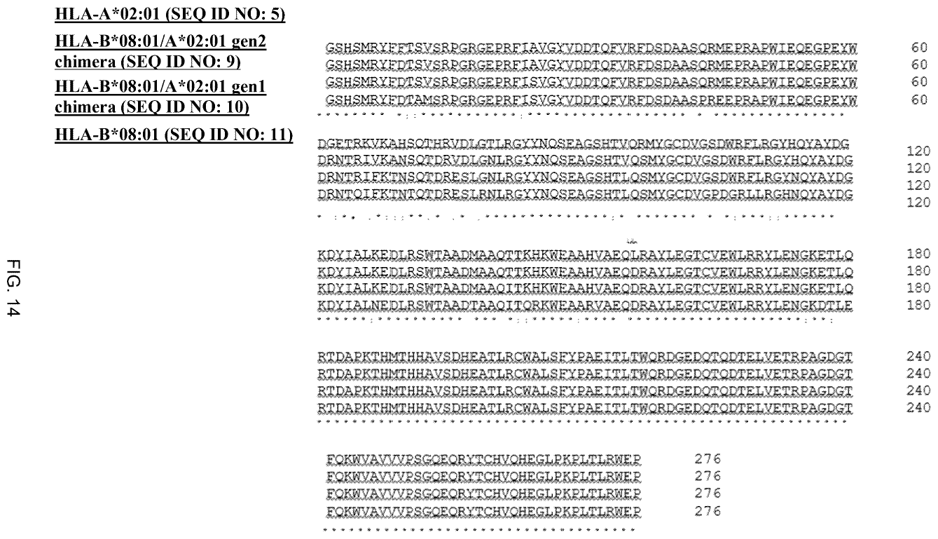

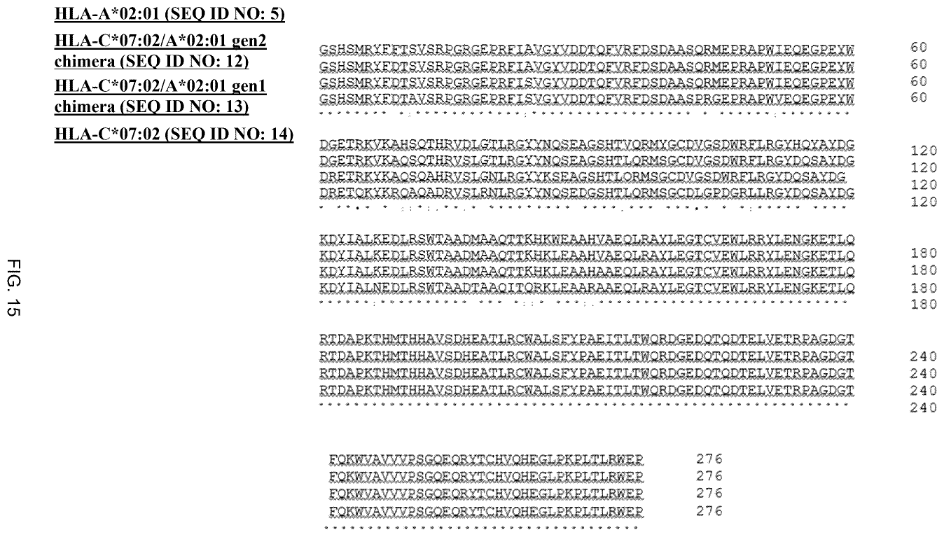

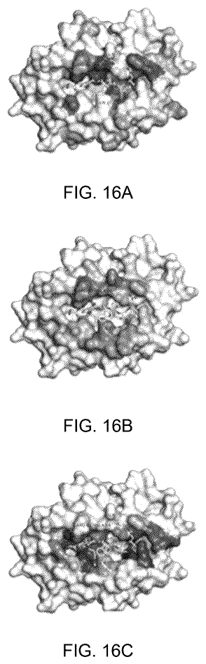

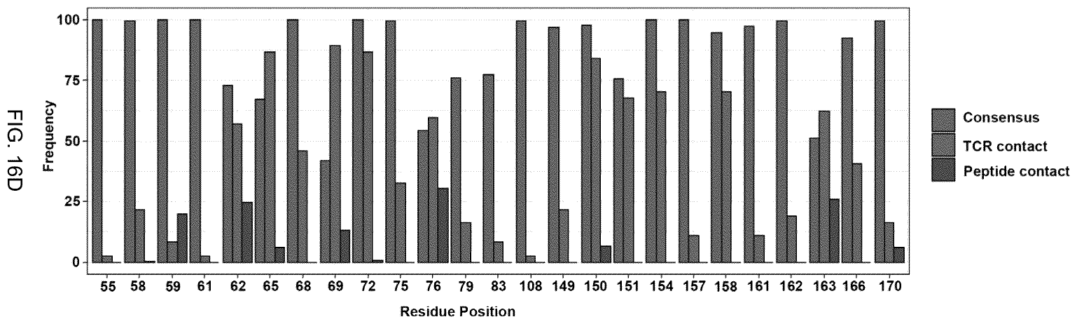

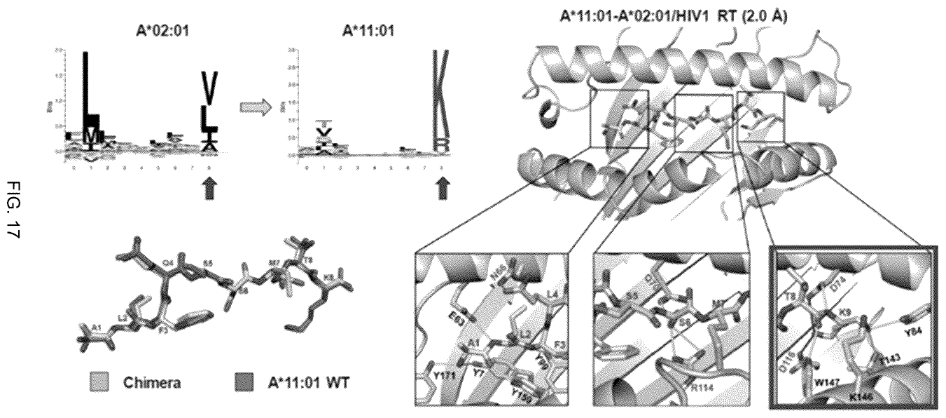

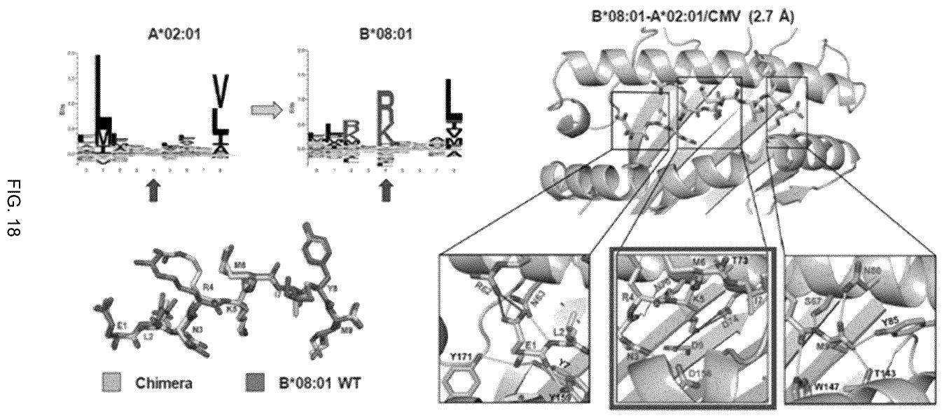

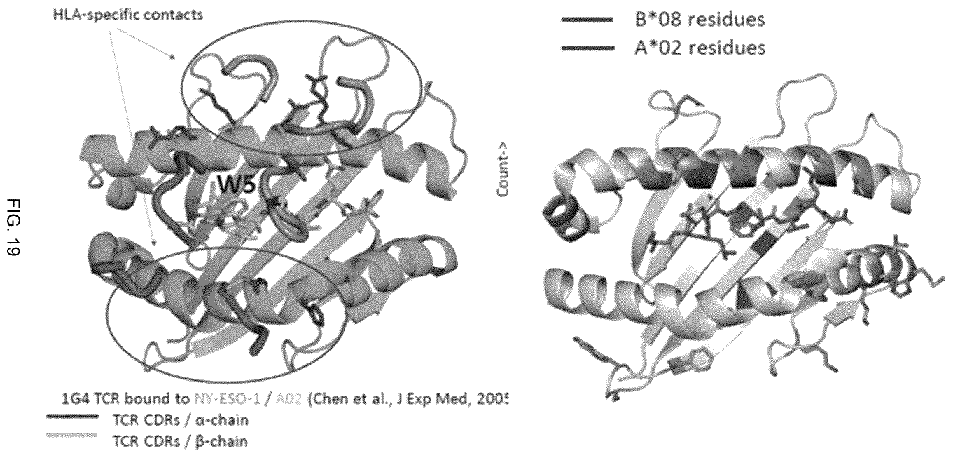

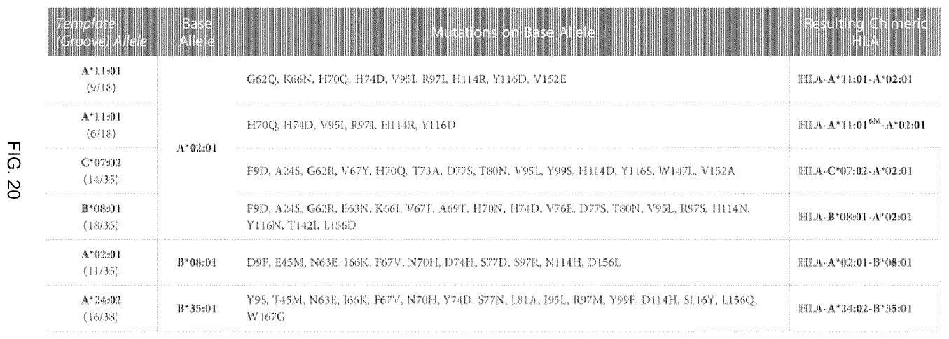

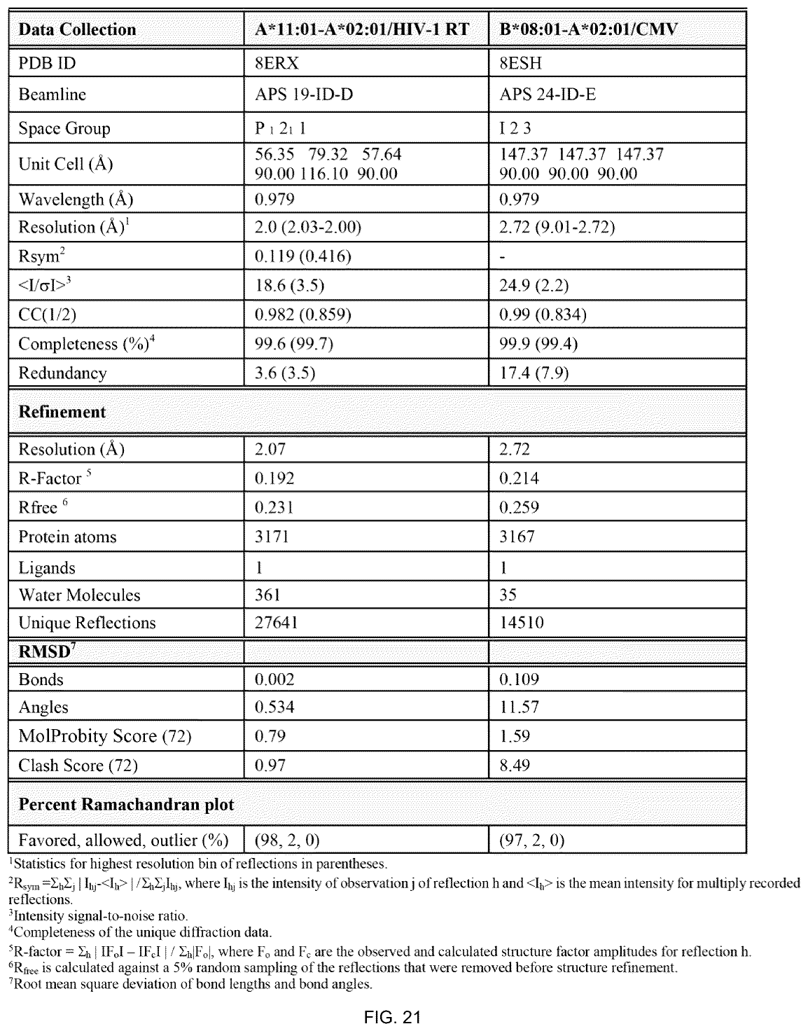

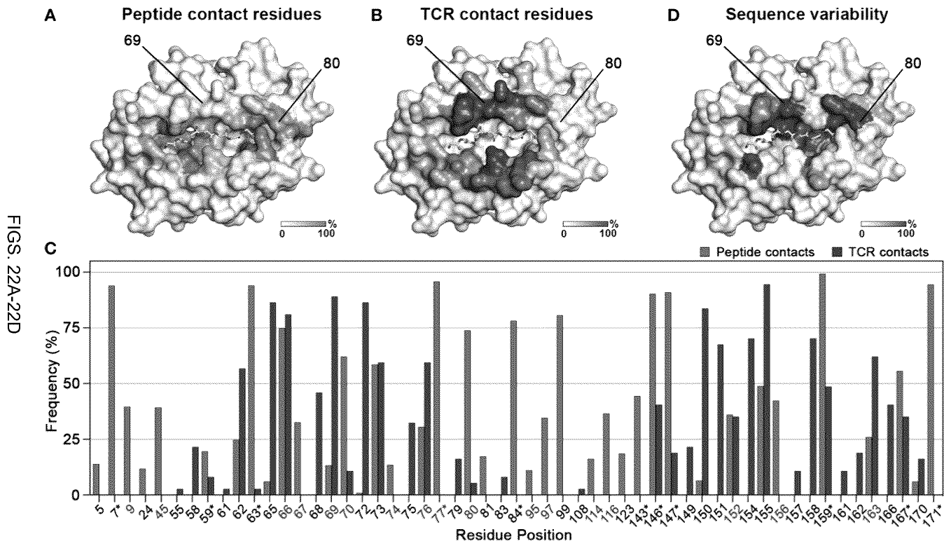

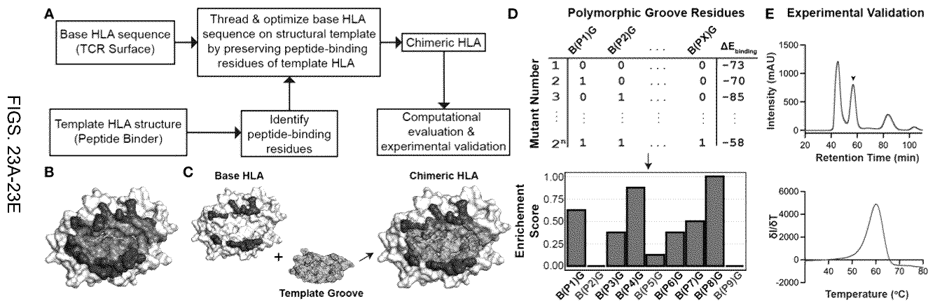

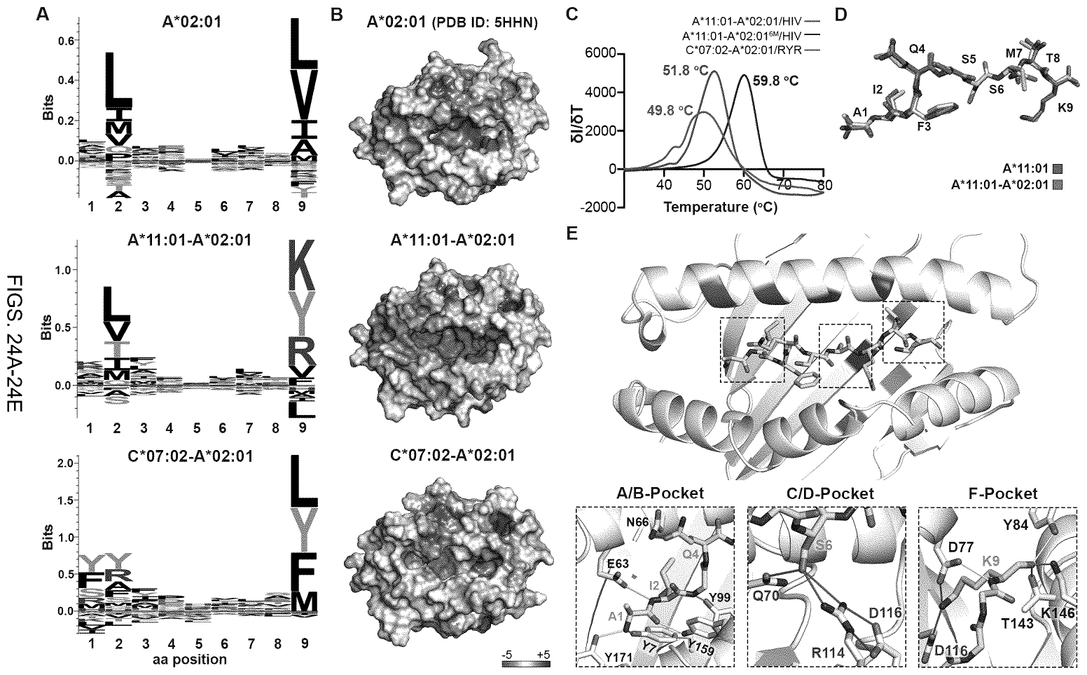

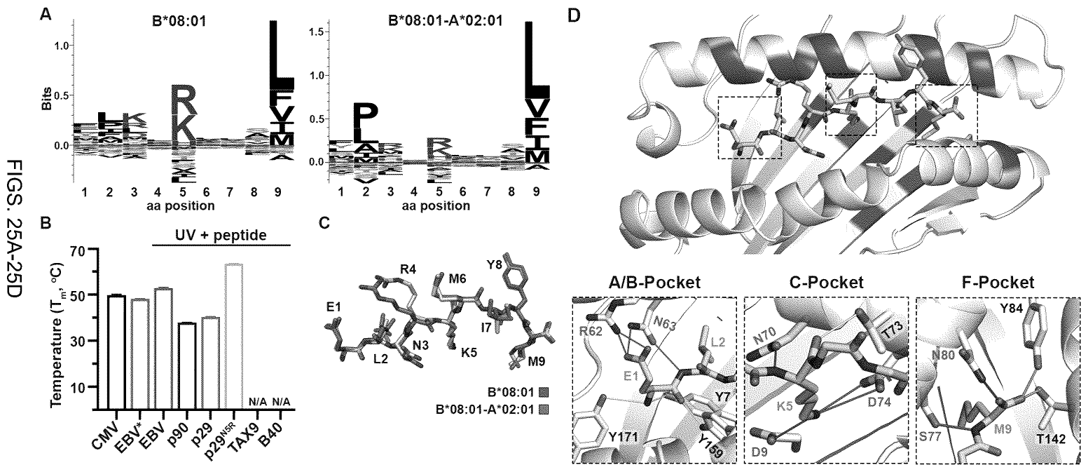

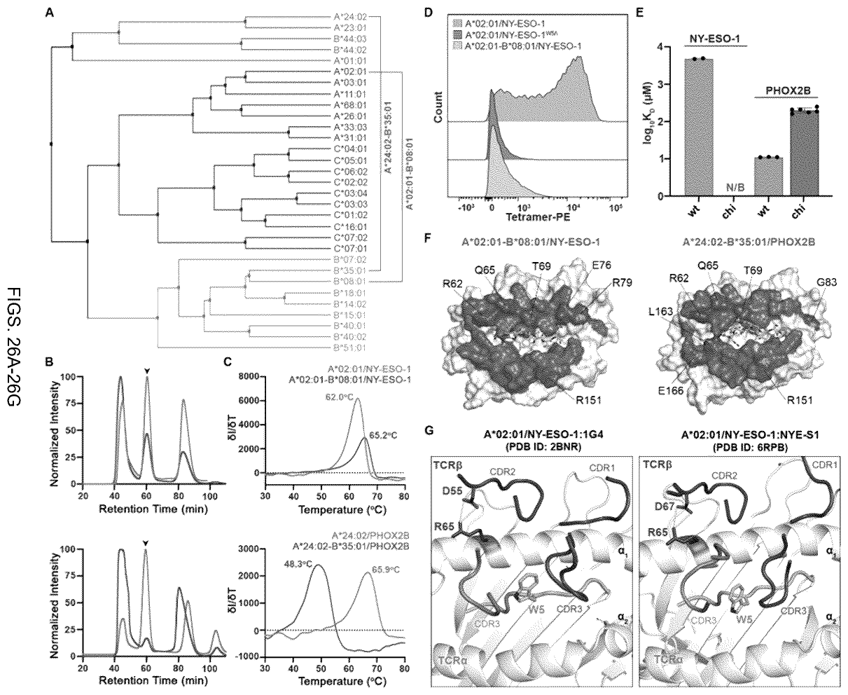

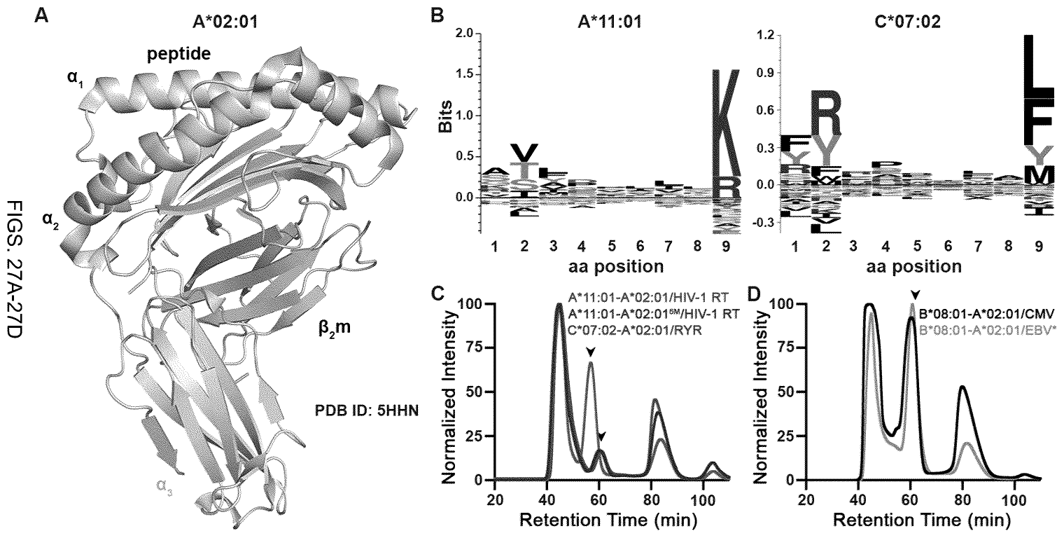

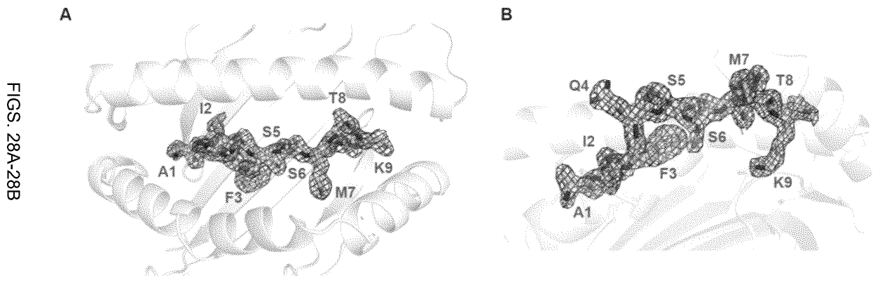

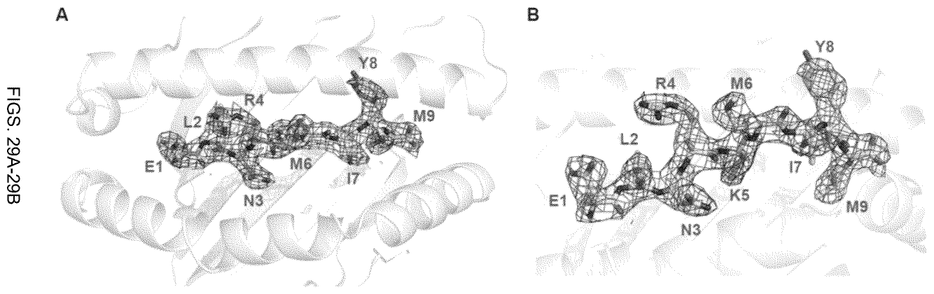

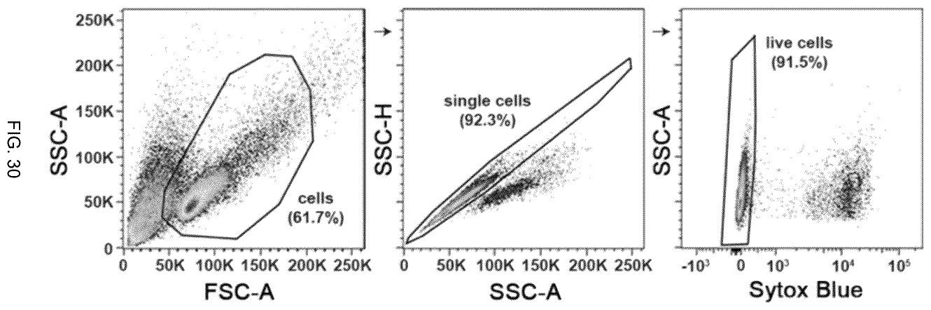

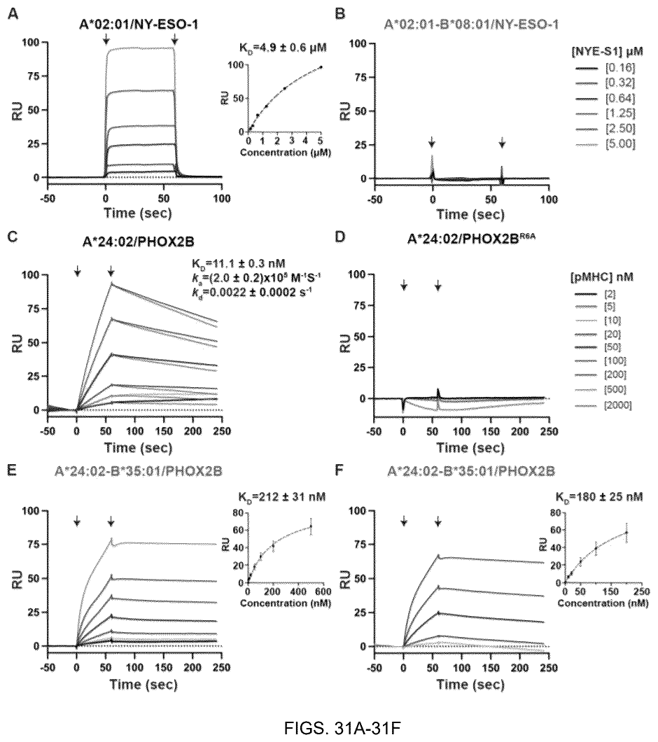

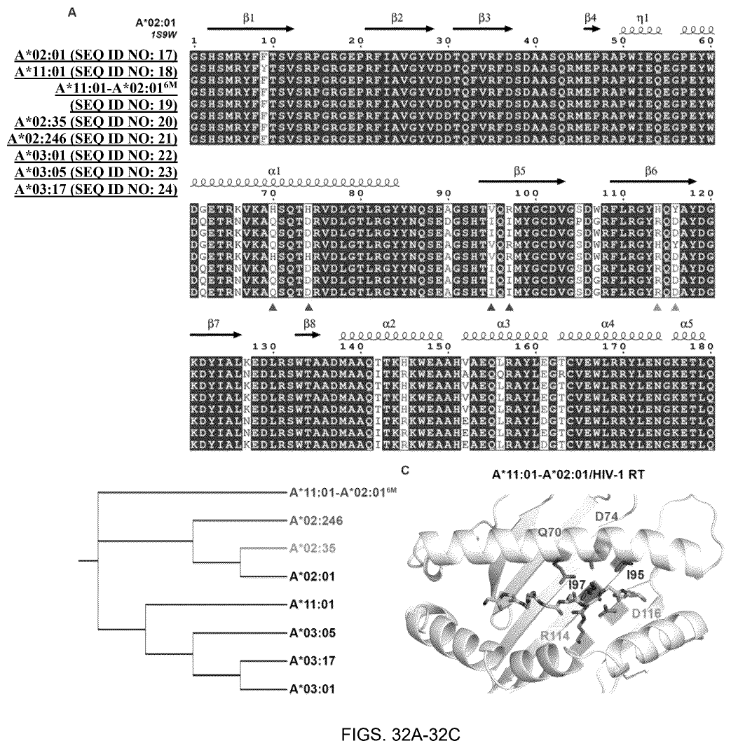

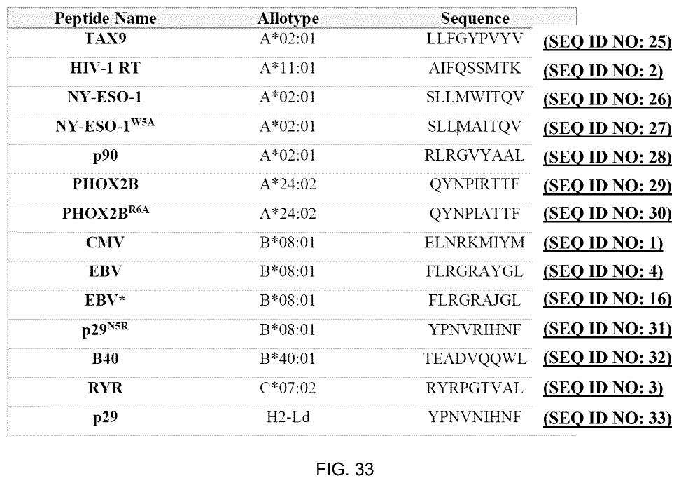

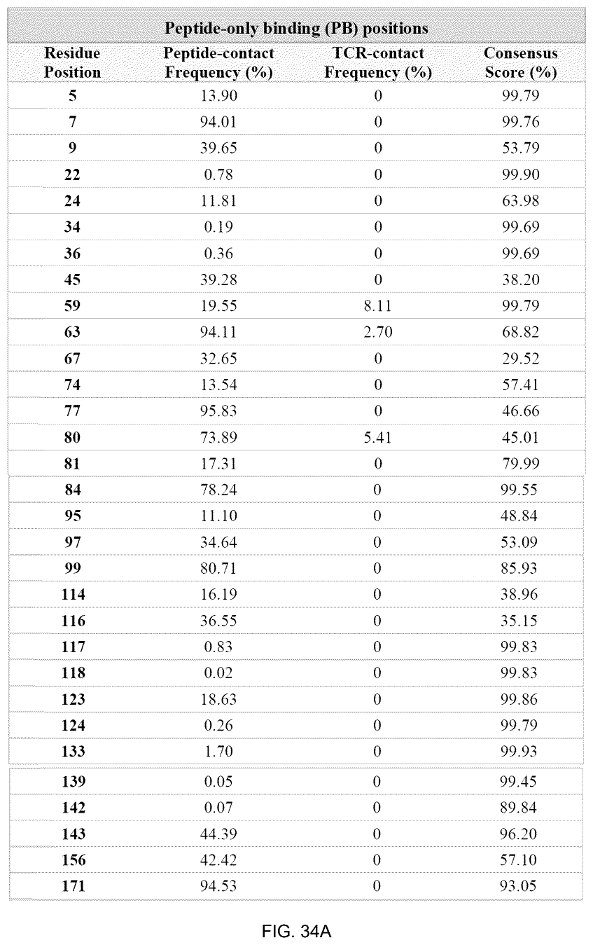

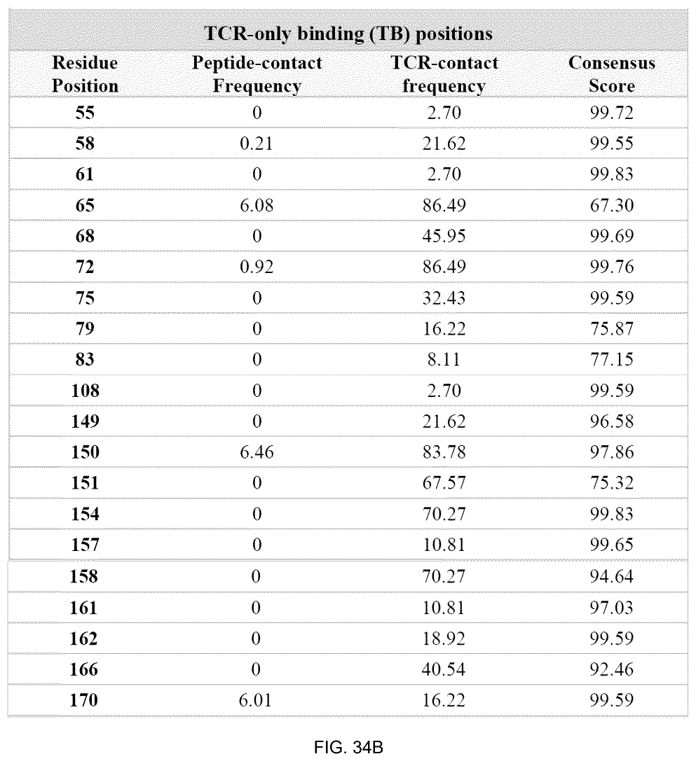

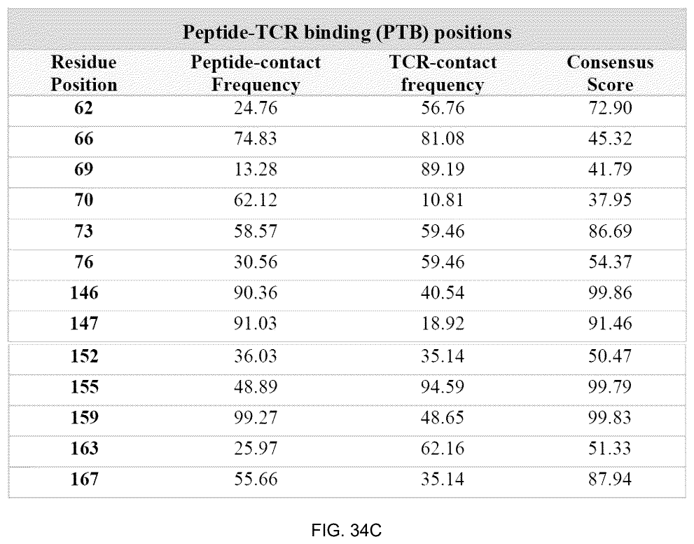

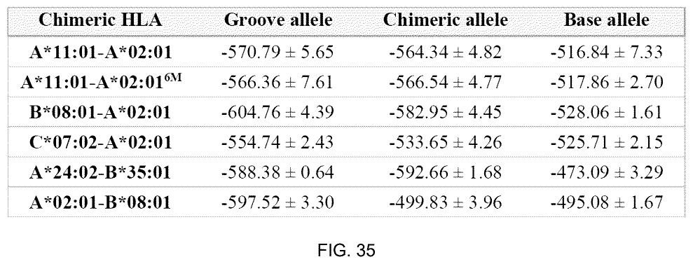

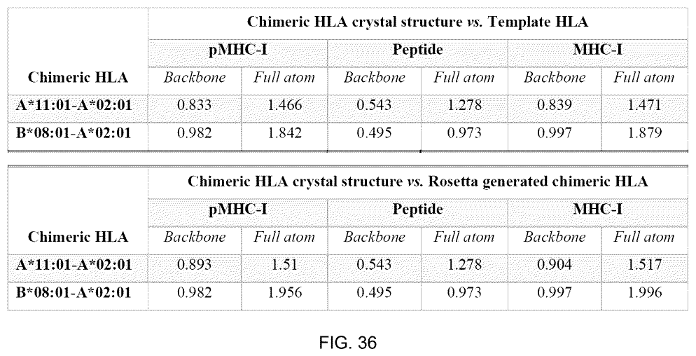

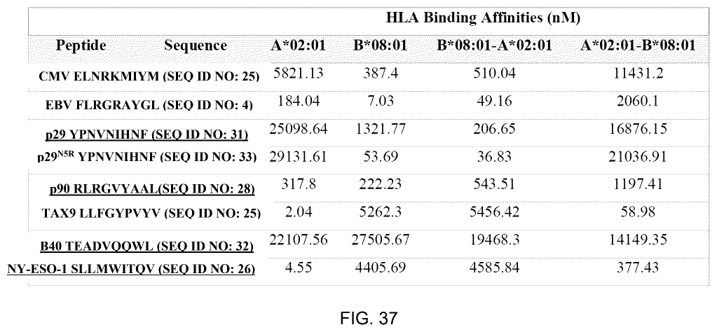

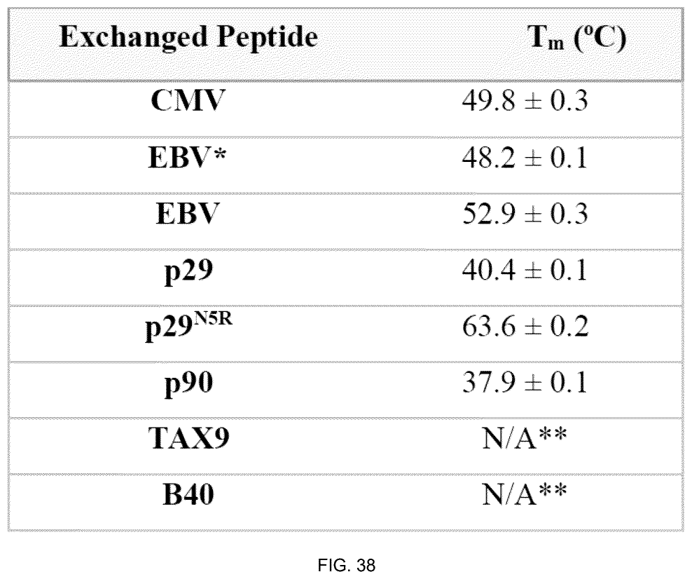

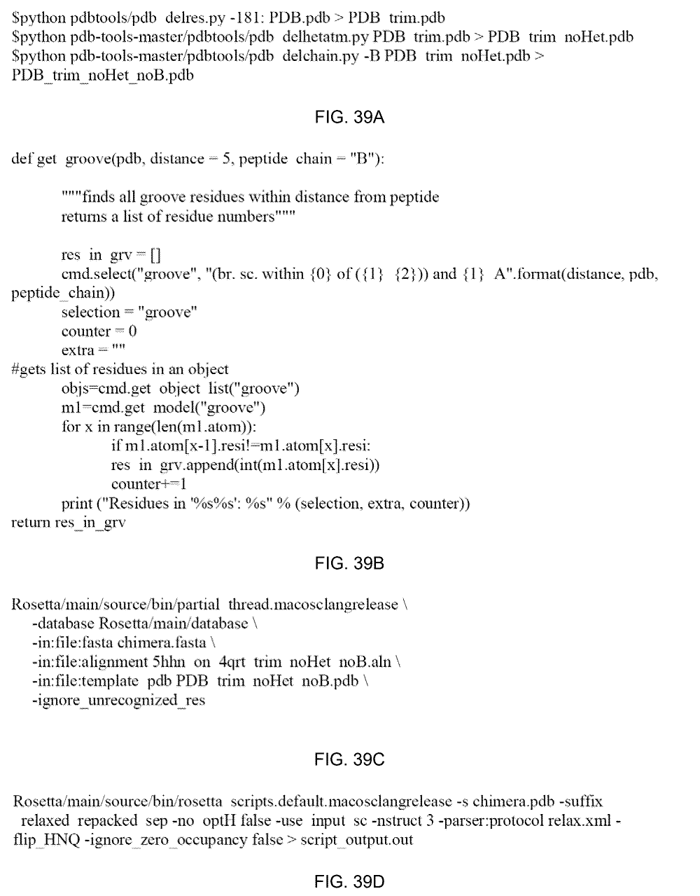

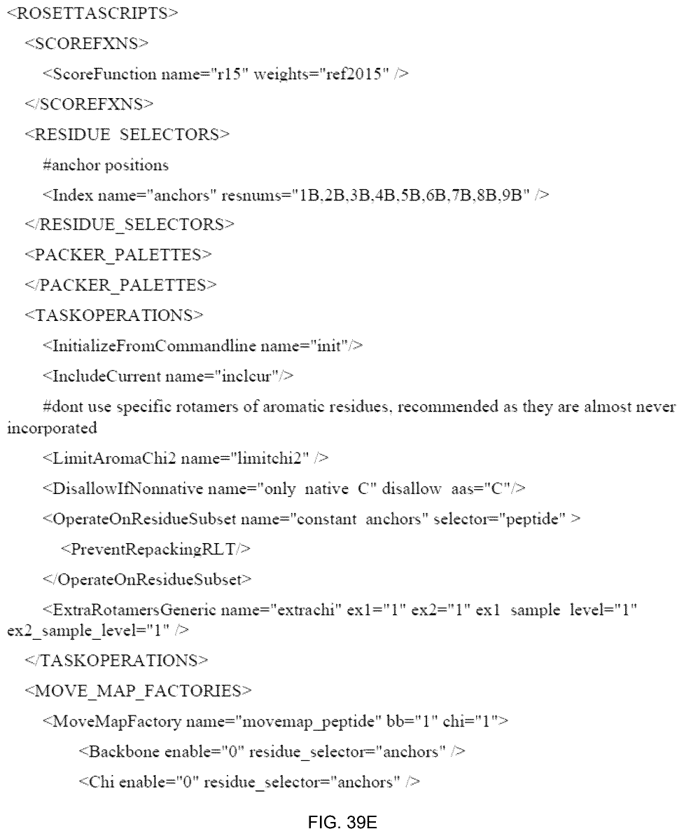

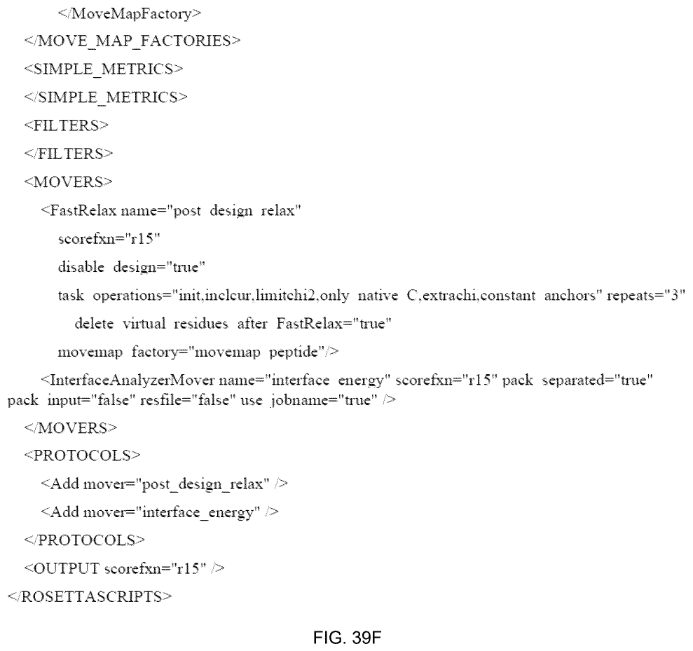

In total, FIG. 1 provides a flow chart of designing MHC chimeras where a designated peptide (ELNRKMIYM; SEQ ID NO: 1) from the groove of the HLA-B*08:01 is selected to be grafted into the groove of a base MHC of HLA-A*02:01. The resulting identification of polymorphic residues responsible for mediating only peptide binding by Rosetta computational analysis, threading, and energetics analysis is then grafted the groove of HLA-A*02:01 to generate the chimeric MHC HLA_B*08:01/A*02:01. A Rosetta-generated structural model of chimeric HLA-B*08:01/HLA-A*02:01 in which polymorphic peptide-HLA-groove residues are grafted from HLA-B*08:01 onto HLA-A*02:01. (bottom) The sequence logo HLA-B*08:01 and HLA-A*02:01 are generated from experimental data (site netMHCpan). The sequence logo of chimeric HLA-B*08:01/HLA-A*02:01 is predicted using netMHCpan4.1.

FIG. 2 Antigen processing and presentation pathway. Proteasomes cut endogenous proteins into peptides which are subsequently transported into the Endoplasmic Reticulum and loaded onto MHC class I via TAP and other chaperons. Loaded pMHCs are then trafficked from the Golgi apparatus and presented at the cell surface.

FIGS. 3A-3C. FIG. 3A The structure of MHC class I depicted schematically. FIG. 3B The structure of MHC class I depicted in PyMol cartoon representation of the crystal structure HLA-B*08:01 (PDB: 4QRT). The domains α1-α3 make up the heavy chain which is bound non-covalently to the β chain. FIG. 3C The peptide binding groove of HLA-B*08:01 bound to the peptide ELNRKMIYM (SEQ ID NO: 1).

FIG. 4 The Sequence Logo of HLA-B*08:01 rendered using Seq2Logo from the netMHCpan database (Reynisson et al., 2020). Anchor positions at P3, P5, and P9 have strong interactions with the peptide binding groove. The peptide binding groove has a preference for peptides with specific residues at the anchor positions (positively charged residues at P3 and P5 and hydrophobic residues at P9 in the case of HLA-B*08:01).

FIG. 5 Functional clustering of the major HLA allotypes. HLA alleles have diverse binding grooves that can accommodate peptides with varying anchor positions including those with a positive P2 (HLA-C*07:02), a positive P5 (HLA-B*08:01), a positive P9 (HLA-A*11:01), a negative P2 (HLA-B*44:05), and the more classical hydrophobic anchors P2 and P9 (HLA-A*02:01). None of the existing alleles can accommodate a negatively charged P2 and a positively charged P9 (Reynisson et al., 2020).

FIG. 6 Potential Applications of Chimeric MHC molecules. Chimeric MHCs can be designed to: 1. Bind novel peptides for biosensing applications (Jones et al., 2006; Ostergaard Pedersen et al., 2001), 2. Break T-cell tolerance and trigger autoreactive T-cell expansion as a potential cancer immunotherapy (Parks et al., 2019). 3. Present a single peptide using single-chain pMHC plasmid engineered into a dendritic cell to expand T-cells in-vivo as a cancer vaccine (Hansen et al., 2010). The chimeras for the last two applications are designed to have the peptide specificity of a native allele to the patient, and the surface structure of a different allele that the patient has.

FIGS. 7A-7C. A*11:01/A*02:01 first and second generation chimera structures. FIG. 7A Sequence logos of HLA-A*02:01 and HLA-A*11:01 with a positive P9 anchor. FIG. 7B A graph showing the peptide binding groove of HLA-A*11:01. Peptide residues are shown as nodes connected by dashed lines corresponding to peptide bonds. Residues that are not polymorphic between the HLA-A*02:01 and HLA-A*11:01 are lighter while polymorphic residues are darker. Solid lines connect the peptide residues to MHC first neighbors (MHC residues with heavy atoms within 3 Å of peptide residue heavy atoms). First neighbors are connected to second neighbors by grey lines. In this case, there is only one polymorphic first neighbor (residue 116). Nodes are labeled by the residues number, 3 letter amino acid code, and the chain of origin. c. HLA-A*11:01/A*02:01 chimera structures. FIG. 7C In both cases, starting with the sequence of A*02:01, mutations were introduced at the positions to match the A*11:01 sequence at that position. The chimera sequence was threaded on the structure of A*11:01 (PDB: 3RL1) then refined using Rosetta's fast_relax protocol. The HIV reverse transcriptase-derived (residues 313-321) peptide (AIFQSSMTK; SEQ ID NO: 2) is darkest.

FIGS. 8A-8C. B*08:01/A*02:01 first and generation chimera structures. FIG. 8A Sequence logos of HLA-A*02:01 and HLA-B*08:01 with a positive P5 anchor. FIG. 8B A graph showing the peptide binding groove of HLA-B*08:01. Peptide residues are shown as nodes connected by dashed lines corresponding to peptide bonds. Residues that are not polymorphic between the HLA-A*02:01 and HLA-B*08:01 are lighter while polymorphic residues are darker. Solid lines connect the peptide residues to MHC first neighbors (MHC residues with heavy atoms within 3 Å of peptide residue heavy atoms). First neighbors are connected to second neighbors by grey lines. Nodes are labeled by the residues number, 3 letter amino acid code, and the chain of origin. Only a subset of the polymorphic sites are needed in the final design of the chimera MHC HLA-B*08:01/A*02:01 chimera structures. FIG. 8C In both cases, starting with the sequence of A*02:01, mutations were introduced at the positions to match the B*08:01 sequence at that position. The chimera sequence was threaded on the structure of B*08:01 (PDB: 4QRT) then refined using Rosetta's fast_relax protocol. The peptide (ELNRKMIYM; SEQ ID NO: 1) is darkest.

FIGS. 9A-9C. C*07:02/A*02:01 first and second generation chimera structures. FIG. 9A Sequence logos of HLA-A*02:01 and HLA-C*07:02 with a positive P2 anchor. FIG. 9B A graph showing the peptide binding groove of HLA-C*07:02. Peptide residues are shown as nodes connected by dashed lines. Residues that are not polymorphic between the HLA-A*02:01 and HLA-C*07:02 are lighter while polymorphic residues are darker. Solid lines connect the peptide residues to MHC first neighbors (MHC residues with heavy atoms within 3 Å of peptide residue heavy atoms). First neighbors are connected to second neighbors by grey lines. Nodes are labeled by the residues number, 3 letter amino acid code, and the chain of origin. Only a subset of the polymorphic sites are needed in the final design of the chimera MHC. FIG. 9C HLA-C*07: 02/A*02:01 chimera structural models. In both cases, starting with the sequence of C*07:02, mutations were introduced at the positions highlighted to match the B*08:01 sequence at that position. The chimera sequence was threaded on the structure of C*07:02 (PDB: 5VGE) then refined using Rosetta's fast_relax protocol. The peptide (RYRPGTVAL; SEQ ID NO: 3) is darkest.

FIG. 10. Schematic outline of the algorithm used for designing chimeric MHCs.

FIGS. 11A-11C. (left panels) SEC purifications of the chimeric peptide/MHC-I complexes. The high affinity peptides used for the refolding are (FIG. 11A) AIFQSSMTK (SEQ ID NO: 2) for HLA-A*11: 01/A*02:01; (FIG. 11B) FLRGRAYGL (SEQ ID NO: 4) for HLA-B*08:01/A*02:01; and (FIG. 11C) RYRPGTVAL (SEQ ID NO: 3) for HLA-C*07: 02/A*02:01. A HiLoad 16/100 Superdex 75 pg column was used and the collected fractions are indicated with arrows. (right panels) DSF progress curves for (FIG. 11A) HLA-A*11: 01/A*02:01; (FIG. 11B) HLA-B*08:01/A*02:01; and (FIG. 11C) HLA-C*07: 02/A*02:01. Arrows indicate the midpoint of the transition and Tm values were determined from the first derivative of the progress curves.

FIG. 11D shows engineered HAL chimeras refold with previously restricted peptides. In the top portion, FIG. 11D presents SEC analysis of HLA chimeras refolded using groove-specific peptides via an Superdex75 16/60 column with arrows pointing to a peak with refolded HLA chimeras (note K in top left sequence, K in top middle sequence, and Y in top right sequence, and correspondence with the peak indicated by the arrow pointing to it). The bottom portion, FIG. 11D presents DSF analysis of assembled and purified HLA chimeras to determine Tm values associated with protein stability.

FIG. 12. Altering MHC-I groove peptide specific through protein design. Structures of the A11/A02 chimera are presented on top with proposed binding on the top left and an X-ray structure on the top right. The prominent amino acid residues (bottom left) and peptide structure (bottom right) are shown on the bottom.

FIG. 13. Sequence alignment of A*02:01 (SEQ ID NO: 5) with A*11:01 (SEQ ID NO: 8), first generation (SEQ ID NO: 7), and second-generation A*11: 01/A*02:01 (SEQ ID NO: 6) chimeras. Residues indicated define the polymorphic sites between A*02:01 and A*11:01 that were mutated from the base MHC (A*02:01) sequence to the groove MHC (A*11:01) sequence to generate the second-generation chimera.

FIG. 14. Sequence alignment of A*02:01 (SEQ ID NO: 5) with B*08:01 (SEQ ID NO: 11), first generation (SEQ ID NO: 10), and second-generation B*08:01/A*02:01 (SEQ ID NO: 9) chimeras. Residues define the polymorphic sites between A*02:01 and B*08:01 that were mutated from the base MHC (A*02:01) sequence to the groove MHC (B*08:01) sequence to generate the second-generation chimera.

FIG. 15. Sequence alignment of A*02:01 (SEQ ID NO: 5) with C*07:02 (SEQ ID NO: 14), first generation (SEQ ID NO: 12), and second-generation C*07: 02/A*02:01 (SEQ ID NO: 13) chimeras. Residues define the polymorphic sites between A*02:01 and C*07:02 that were mutated from the base MHC (A*02:01) sequence to the groove MHC (C*07:02) sequence to generate the second-generation chimera.

FIGS. 16A-16D shows that the pMHC structure presents distinct surfaces for interactions with T-Cell Receptors (TCRs) and peptides.

FIG. 17 shows A02 engineered A11 groove specificity recapitulates (or repeats or maintains) the original peptide conformation (three-dimensional conformation), demonstrating that chimera of the invention have fidelity as to three-dimensional conformation.

FIG. 18 shows A02 engineered B08 groove specificity recapitulates (or repeats or maintains) the original peptide conformation (three-dimensional conformation), demonstrating that chimera of the invention have fidelity as to three-dimensional conformation.

FIG. 19 shows that tetramerized A02/B08 chimeras can probe the HLA allele dependence of TCR interactions, demonstrating the utility of the present invention for screening for reagents that interact with residues of the complex in comparison with residues from the peptide (such that in peptide-centric therapeutics, e.g., products from any sort of system, e.g., phage display, yeast display, directed evolution, can be screened using tetramerized chimeras of the invention as they recognize peptide-MHC moiety irrespective of the peptide).

FIG. 20 Summary of amino acid substitutions introduced in the sequence of a base allele to derive chimeric HLAs. The number of amino acid substitutions introduced in the sequence of the base allele, versus the total number of polymorphic residues between the template (groove) and base alleles, are shown in brackets.

FIG. 21 Crystallography data collection and refinement statistics for the HLA-A*11:01-A*02:01/HIV-1 RT and B*08:01-A*02:01/CMV chimeras.

FIGS. 22A-22D Contiguous molecular surfaces defined by polymorphic HLA residues mediate interactions with peptides, and TCRs. The calculated (FIG. 22A) peptide-contact, and (FIG. 22B) TCR-contact frequencies of the first 180 amino acids are highlighted in a low and high gradient, respectively. The structure of HLA A*02:01 (PDB ID: 1S9W) was used as template. (FIG. 22C) Bar graph of the peptide- and TCR-contact frequencies for positions with at least one value higher than 2%. Polymorphic positions with a consensus score below 60% are highlighted, while residues that form conserved hydrogen bond networks with the peptide main chain are marked with an asterisk (*) (52). (FIG. 22D) Sequence variability for each position P plotted as (100-Consensus score) on the HLA-A*02:01 structure, from a low to high gradient.

FIGS. 23A-23E. General workflow for generating chimeric HLA molecules using a fixed-backbone, structure-guided approach. (FIG. 22A) The general workflow of the CHaMeleon approach to generate chimeric MHC-I molecules. (FIG. 22B) The structure of HLA-A*02:01 bound to the peptide SLLMWITQC (PDB ID: 1S9W; SEQ ID NO: 15) where the peptide-only, TCR-only and peptide-TCR-binding residues are highlighted. (FIG. 22C) Grafting the peptide-biding groove of a template onto a base allele to create chimeric molecules. TCR-only positions are highlighted and peptide-only or peptide-TCR-binding residues are highlighted. The structure of HLA-A*02:01 (PDB ID: 1S9W) was used as an example. (FIG. 22D) Exhaustive combinatorial sampling of groove allele substitutions on the base allele and binding energy calculations was performed to evaluate the chimeric HLA models. The top 2.5% of structures with lowest binding energies were used to calculate Enrichment Scores at each polymorphic position (PX) in the groove, which represents the fraction of chimeric HLAs with a specific mutation from base (B) to groove (G) allele residue. Positions with 0 or very low enrichment scores are highlighted. (FIG. 22E) Experimental validation of the chimeric pMHC-I by size exclusion chromatography (SEC; top). The protein peak is indicated by the arrow (57.5 min), while the additional peaks correspond to protein aggregates (47 min) and free B2m (84 min). Thermal stability of the purified molecules was assessed using differential scanning fluorimetry (DSF; bottom) experiments.

FIGS. 24A-24E. Production of chimeric HLA-A*02:01 peptide complexes with altered B- or F-pocket specificities according to A*11:01 or C*07:02 structural templates. (FIG. 24A) Sequence logos of HLA-A*02:01, A*11:01-A*02:01, and C*07:02-A*02:01 molecules rendered using an in-house protocol and visualized in Seq2Logo from the NetMHCpan4.0 (40). (FIG. 24B) Electrostatic surface potential analysis for HLA-A*02:01 (PDB ID: 5HHN), A*11:01-A*02:01, and C*07:02-A*02:01 calculated using the APBS solver in PyMOL. In all panels, the electrostatic surface potential is shown as a range between +5 kT/e to −5 kT/e representing positive and negative charges, respectively. kB. Boltzmann constant; T, temperature; e, unit charge. (FIG. 24C) Thermal stabilities of HLA-A*11:01-A*02:01/HIV-1 RT, A*11:01-A*02: 016M/HIV-1 RT, and C*07:02-A*02:01/RYR. Data are mean±SD obtained for n=3 technical replicates. (FIG. 24D) Overlay of the HIV-1 RT peptide bound to the chimeric HLA-A*11:01-A*02:01 and wild-type A*11:01 molecules. (FIG. 24E) Crystal structure of the HLA-A*11:01-A*02:01/HIV-1 RT complex. Substitutions of the HLA-A*11:01-A*02:01 and A*11:01-A*02: 016M chimeras are highlighted. Hydrogen bonds and salt bridges between the peptide and the base or groove allele residues are represented by lines. Peptide, A*02:01-specific, and A*11:01-specific residues are labeled.

FIGS. 25A-25D. Introduction of a P5 anchoring specificity into the C-pocket of HLA-A*02:01 using a B*08:01 structural template. (FIG. 25A) The sequence logos of the HLA-B*08:01 (left), and B*08:01-A*02:01 (right) rendered using an in-house method and visualized in Seq2Logo from the NetMHCpan4.0 (40). (FIG. 25B) Thermal stabilities of HLA-B*08:01-A*02:01 refolded with CMV (ELNRKMIYM; SEQ ID NO: 1) or EBV* (FLRGRAXGL (SEQ ID NO: 16), where X is the 3-amino-3-(2-nitrophenyl)-propionic acid) and after UV-irradiation in the presence of 10-fold molar excess of EBV, p90, p29, p29^{N5R}, TAX9 (SEQ ID NO: 25), and B40 peptides. Data are mean±SD obtained from n=3 technical replicates. N/A, no exchange. (FIG. 25C) Overlay of the CMV peptide bound to the chimeric B*08:01-A*02:01 and wild-type B*08:01 molecules. (FIG. 24D) Crystal structure of HLA-B*08:01-A*02:01/CMV complex where substitutions of the groove residues are highlighted. Hydrogen bonds and salt bridges between the peptide and the base or groove allele residues are shown as lines. Peptide, A*02:01-specific, and B*08:01-specific residues are labeled.

FIGS. 26A-26G. Application of chimeric HLAs as probes for assessing peptide-centric interactions with immune receptors for targeted therapy. (FIG. 26A) Phylogenetic analysis of a divergent set of common HLA allotypes using the TCR contacting residues to define sequence similarity. (FIG. 26B) SEC traces of recombinant HLA-A*02:01/NY-ESO-1, A*02:01-B*08:01/NY-ESO-1, A*24: 02/PHOX2B, and A*24:02-B*35: 01/PHOX2B molecules. The protein peaks are indicated by the arrows. (FIG. 26C) Melting temperatures (Tₘ, ° C.) of the pMHC alleles in (FIG. 26B) determined by DSF experiments. Data are mean±SD obtained for n=3 technical replicates. (FIG. 26D) Staining of 1G4-transduced primary CD8+ T cells with PE-conjugated tetramers of A*02:01 presenting the wild-type NY-ESO-1 or the mutated NY-ESO-1^{W5A} peptides, and the chimeric A*02:01-B*08:01/NY-ESO-1 complex. Staining was observed only in the case of A*02:01/NY-ESO-1, suggesting positive recognition by the TCR, whereas in the case of the negative control and the chimeric pMHC the interactions are disrupted. (FIG. 26E) Comparison of the SPR determined K_{D} values for NYE-S1 and 10LH interacting with HLA-A*02:01 and A*24:02 wild-type and chimeric molecules presenting NY-ESO-1 and PHOX2B peptides, respectively. Data are mean±SD for n=2 (NYE-S1) or n=3 (10LH) technical replicates. K_{D}, equilibrium constant; N/B, no binding. (FIG. 26F) Surface structure of the Rosetta model of HLA-A*02:01-B*08:01/NY-ESO-1 and A*24:02-B*35: 03/PHOX2B chimeras, where all TCR-contact residues are highlighted. The wild-type B*08:01 or B*35:01 residues are shown. (FIG. 26G) Structural comparison of the HLA-A*02:01/NY-ESO-1 complex bound by the TCRs 1G4 (PDB ID: 2BNR) and NYE-S1 (PDB ID: 6RPB). The HLA-A*02:01, the NY-ESO-1 peptide, and the TCR-α and -β chains are colored. The identified Arg65 is represented as a single stick and its interactions with TCR-β chain are shown.

FIGS. 27A-27D. Design and experimental validation of chimeric HLA-A*02:01 peptide complexes. (FIG. 27A) Crystal structure of HLA-A*02:01 where the α1-3 domains of the heavy chain, the light chain β2m and the peptide are depicted (PDB ID: 5HHN). (FIG. 27B) Sequence logo of peptides specific for HLA-A*11:01 (left), and HLA-C*07:02 (right), rendered using Seq2Logo in NetMHCpan4.0 (Thomsen MCF, Nielsen M. Seq2Logo: a method for construction and visualization of amino acid binding motifs and sequence profiles including sequence weighting, pseudo counts and two-sided representation of amino acid enrichment and depletion. Nucleic Acids Res (2012) 40: W281-287. doi: 10.1093/nar/gks469). SEC traces of (FIG. 27C) HLA-A*11:01-A*02:01, HLA-A*11:01-A*02: 016M refolded with the HIV-1 RT (AIFQSSMTK; SEQ ID NO: 2) peptide, C*07:02-A*02:01 refolded with the RYR (RYRPGTVAL; SEQ ID NO: 3) peptide, and (FIG. 27D) HLA-B*08:01-A*02:01 chimera refolded with the CMV (ELNRKMIYM; SEQ ID NO: 1) and EBV* (FLRGRAXGL; SEQ ID NO: 16) peptides. The protein peaks are indicated by the arrows. X, 3-amino-3-(2-nitrophenyl)-propionic acid.

FIGS. 28A-28B. Structure of the HIV-1 RT peptide presented by the HLA-A*11:01-A*02:01 chimera. (FIGS. A and B) HLA-A*11:01-A*02:01 2Fo-Fc omit maps (blue) around the HIV-1 RT peptide (AIFQSSMTK; yellow; SEQ ID NO: 2) contoured at 1.3 sigma.

FIGS. 29A-29B. Structure of the CMV peptide presented by the HLA-B*08:01-A*02:01 chimera. (FIGS. A and B) HLA-B*08:01-A*02:01 2Fo-Fc omit maps around the CMV peptide (ELNRKMIYM; SEQ ID NO: 1) contoured at 1.0 sigma.

FIG. 30. Flow cytometry gating strategy of CD8+ T cells transduced with 1G4. Previously transduced or non-transduced primary human CD8+ T cells were thawed and recovered prior to tetramer staining. Cells were first sorted by side and forward scatter (SSC-A and FSC-A) followed by single cell isolation (SSC-A versus SSC-H plot). Gating for live cells was determined by Sytox blue staining and transduction efficiency was determined by staining with an anti-Vβ13.1-APC antibody (Miltenyi Biotec). Gates are shown in black and the percentages of events that are gated in parentheses. Acquisition was performed on LSR Fortessa (BD), and the data analyzed by FlowJo v10.8.1.

FIGS. 31A-31F. TCR recognition dependence on interactions with MHC-I framework residues compared to peptide-centric CARs. SPR sensorgrams of vary concentrations of soluble NYE-S1 receptor flowed over a streptavidin chip coupled with (FIG. A) A*02:01/NY-ESO-1 and (FIG. B) A*02:01-B*08:01/NY-ESO-1. SPR sensorgrams of various concentrations of (FIG. C) A*24: 02/PHOX2B or (FIG. D) A*24: 02/PHOX2BR6A as negative control flowed over a streptavidin chip coupled with the biotinylated scFv 10LH. Fits from the kinetic analysis are shown with red lines. (FIG. E) and (FIG. F) Two independent replicates of chimeric A*24:02-B*35: 01/PHOX2B complex flown over a streptavidin chip conjugated with biotinylated 10LH. The KD is an estimation since binding saturation is not reached. Data are mean±SD for n=2 (FIGS. A, B), n=3 (FIGS. C-F) technical replicates. Injection and washing start points are indicated by arrows. KD, equilibrium dissociation constant; ka, association rate constant; kd, dissociation rate constant; RU, resonance units.

FIGS. 32A-32C. Convergent evolution of A02 and A03 supertypes. (FIG. A) Sequence alignment of the α1 and α2 helices (residues 1-180) of A*11:01-A*02: 016M with the template A*11:01, the base A*02:01, and the most similar to the chimeric molecules, known HLA allotypes. Substituted residues of the A*11:01-A*02: 016M chimera that are different in A*02:246, A*02:35, or both are shown with arrowheads. Alignments were performed in ClustalOmega (Sievers F, Wilm A, Dineen D, Gibson T J, Karplus K, Li W, Lopez R, McWilliam H, Remmert M, Söding J, et al. Fast, scalable generation of high-quality protein multiple sequence alignments using Clustal Omega. Mol Syst Biol (2011) 7:539. doi: 10.1038/msb.2011.75) and processed in ESPript (Robert X, Gouet P. Deciphering key features in protein structures with the new ENDscript server. Nucleic Acids Res (2014) 42: W320-324. doi: 10.1093/nar/gku316). The structure of HLA-A*02:01 (PDB ID: 199W) was used as reference. Conserved residues are in boxes. (FIG. B) Phylogenetic tree based on the sequence alignment in (A), using the Maximum Likelihood method in MEGA7 (Kumar S, Stecher G, Tamura K. MEGA7: Molecular Evolutionary Genetics Analysis Version 7.0 for Bigger Datasets. Molecular Biology and Evolution (2016) 33:1870-1874. doi: 10.1093/molbev/msw054) and processed in iTOL (Letunic I, Bork P. Interactive Tree Of Life (iTOL) v5: an online tool for phylogenetic tree display and annotation. Nucleic Acids Research (2021) 49: W293-W296. doi: 10.1093/nar/gkab301). (FIG. C) The crystal structure of HLA-A*11:01-A*02:01/HIV-1 RT chimera where the indicated residues in (FIG. A) are highlighted, respectively.

FIG. 33. Summary of the peptides used in Example 4.

FIGS. 34A-34C. Summary of the peptide-contact and TCR-contact frequencies (%) for each position P, as calculated from analysis of 384 pMHC-I structures from the Protein Data Bank (HLA3DB; https://hla3db.research.chop.edu/) and 36 pMHC-TCR structures from the ATLAS database (Borrman T, Cimons J, Cosiano M, Purcaro M, Pierce B G, Baker B M, Weng Z. ATLAS: A database linking binding affinities with structures for wild-type and mutant TCR-pMHC complexes: Linking TCR-pMHC Affinities with Structure. Proteins (2017) 85:908-916. doi: 10.1002/prot.25260). Peptide-only binding (PB) positions have a non-zero peptide-contact frequency and a TCR-contact frequency less than 10%, TCR-only binding (TB) positions have a non-zero TCR-contact frequency and a peptide-contact frequency less than 10%, and peptide-TCR-binding (PTB) have both frequencies greater than 10%. The consensus score for each category was calculated from the analysis of 2896 sequences HLAs curated from the IMGT/HLA database (Robinson J. IMGT/HLA and IMGT/MHC: sequence databases for the study of the major histocompatibility complex. Nucleic Acids Research (2003) 31:311-314. doi: 10.1093/nar/gkg070).

FIG. 35. Total binding energy calculations for the base, template, and chimeric alleles threaded and relaxed through the template allele. The mean energy values from three independent models and the standard deviation are included. Structures were calculated using the Rosetta software (Leman J K, Weitzner B D, Lewis S M, Adolf-Bryfogle J, Alam N, Alford R F, Aprahamian M, Baker D, Barlow K A, Barth P, et al. Macromolecular modeling and design in Rosetta: recent methods and frameworks. Nat Methods (2020) 17:665-680. doi: 10.1038/s41592-020-0848-2).

FIG. 36. RMSD (Å) values for the solved crystal structures vs. the template allele and vs. the Rosetta model. The calculations were performed using the ‘Super’ command in PyMOL, with the appropriate residue selectors for the backbone and full atom and 0 refinement cycles.

FIG. 37. Summary of the peptides used for the HLA-B*08:01-A*02:01 UV-mediated exchange experiments. Binding affinities (nM) to the template, base, and chimeric molecules were calculated using NetMHCpan4.0 (Reynisson B, Alvarez B, Paul S, Peters B, Nielsen M. NetMHCpan-4.1 and NetMHCIIpan-4.0: improved predictions of MHC antigen presentation by concurrent motif deconvolution and integration of MS MHC eluted ligand data. Nucleic Acids Res (2020) 48: W449-W454. doi: 10.1093/nar/gkaa379).

FIG. 38. Summary of the Tm (oC) calculations for HLA-B*08:01-A*02:01 refolded with the CMV, EBV* (EBV*=FLRGRAXGL (SEQ ID NO: 16), where X is the 3-amino-3-(2-nitrophenyl)-propionic acid) and the UV-mediated peptide exchange experiments. **N/A, no exchange.

FIG. 39A. Preprocessing of a sample PDB file (PDB_ID.pdb)

FIG. 39B. Pymol script for identifying the peptide binding groove

FIG. 39C. Threading a protein sequence through a protein structure

FIG. 39D Command for relaxing a protein structure and calculating total and binding energies

FIGS. 39E-39F. Rosetta scripts relax.xml file

Appendix 1 and Appendix 2, with reference to FIGS. 17 and 18 provide coordinates for the crystal structures.

Appendix 3, with reference to FIGS. 17, 18 and 19 provides sequences of the chimera designs with differential scanning fluorimetry analysis of SEC-purified chimera, and differential scanning fluorimetry analysis of SEC-purified chimera.

### DETAILED DESCRIPTION OF THE INVENTION

The present invention is described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. One skilled in the art will recognize that the systems and devices of embodiments of the invention can be used with any of the methods of the invention and that any methods of the invention can be performed using any of the systems and devices of the invention. Embodiments comprising various features may also consist of or consist essentially of those various features. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Unless otherwise defined, all technical and scientific terms used herein have the same meaning as would be commonly understood or used by one of ordinary skill in the art encompassed by this technology and methodologies.

### Outline of the CHaMeleon Approach

### Terminology:

Groove allele: an MHC-I allele that presents a specific set of peptides, characterized in compact form by a sequence logo.

Base allele: a MHC-I allele that has a specific outside surface structure, excluding the surface defined by its peptide binding grove.

Anchor N MHC-I allele: a MHC-I allele that has a specific anchor position motif at peptide position N.

Crystal structure: a high-resolution X-ray structure of a particular peptide: MHC complex that has been downloaded from the Protein Data Bank (Berman et al., 2000).

General situation definition: To alter the peptide binding specificity of a given base MHC-I allele such that it can bind and display a new set of peptides.

More specific instance: Given a base allele with a defined surface structure and a groove allele with a specific peptide specificity, change the peptide specificity of the base allele to that of the groove allele.

Exemplar application: Given the structure of a groove allele bound to a peptide P in a conformation C, and a base allele that does not bind peptide P at all or it does not bind peptide P in conformation C, introduce a series of amino acid substitutions in the peptide binding groove of the base allele such that it can bind peptide P in conformation C.

To demonstrate an exemplar application, Applicants define as a base allele the sequence of a common human MHC-I allele in multiple ethnic groups, HLA-A*02:01, with a peptide logo that contains amino acids with hydrophobic side chains at the anchor positions P2 and P9 (FIG. 7A). Applicants then selected a set of 3 different examples of groove alleles, H {HLA-A*11:01, HLA-B*08:01, HLA-C*07:02} (FIGS. 7-9), each bound to a unique peptide sequence (P1 . . . . P3) in conformations (C₁ . . . . C₃) as observed in high-resolution crystal structures (X₁ . . . . X₃) from the PDB. The set of groove alleles H is chosen such that they possess unique peptide logos, different from HLA-A*02:01. It is worth noting that HLA-A*02:01 does not bind to any of the peptides P1-P3.

Methods. Applicants propose the following two alternative methods to generate 1^{st} and 2^{nd} generation chimeras between HLA-A*02:01 and each allele in the set H, as outlined in detail below and in FIG. 10. The design of 1^{st}- and 2^{nd}-generation chimeras is based on the same principle of substituting amino acids in the groove of a base MHC-I allele to match the peptide specificity of a groove MHC-I allele, however the 2^{nd}-generation designs use a more restricted set of substitutions.

### First Generation Chimeras (First Embodiment for Generation of Chimera):

1. Using a sequence alignment between the groove MHC-I allele and the base MHC-I allele, Applicants identify all polymorphic positions which differ between the sequences of the two alleles (See step II in Example I, implementation, and software).

2. Using the crystal structure of the groove MHC-I allele bound to peptide P in conformation C, Applicants select all residues in the groove MHC-I allele's sequence that have at least one sidechain heavy atom within 5 Å of a peptide heavy atom. Applicants term this set of residues peptide contact residues (See step II in Example I, implementation and software).

3. Applicants identify the sub-set of polymorphic positions which also correspond to peptide contact residues in the sequence of the groove MHC-I allele. Applicants term this set polymorphic contact residues, defined in the sequence of the base allele (See step II in Example I, implementation, and software).

4. A 1^{st}-generation chimera between the base and groove alleles is defined by the amino acid sequence of the base allele where all positions corresponding to polymorphic contact residues are mutated to the equivalent residues in the groove allele (See step III in Example I, implementation, and software).

5. To produce a structural model of the chimera, Applicants use a computer software to thread the chimera MHC-I sequence onto the groove MHC-I allele structure, then locally refine the generated structure to find an energy minimum which corresponds to the stable folded state of the protein. Applicants use the model of the chimeric structure to evaluate its stability and peptide binding free energy, and compare it with those derived from the groove MHC X-ray structure (See step IV in Example I, implementation and software).

Representative designed sequences for 3 exemplar chimeras (HLA-A*11: 01/A*02:01, HLA-B*08:01/A*02:01, HLA-C*07: 02/A*02:01) were generated by gene synthesis, expressed in E. coli, and assembled into heterotrimeric MHC-I protein complexes with their respective target peptides and β₂m light chains using in vitro refolding, followed by gel filtration purification. Further validation by differential scanning fluorimetry shows that all three designs yield properly conformed, stable MHC-I molecules with melting temperature (Tₘ) values in the expected range for MHC-I molecules (51° C. for HLA-A*11: 01/A*02:01, 52° C. for HLA-B*08:01/A*02:01 and 48° C. for HLA-C*07: 02/A*02:01). These results are shown in FIG. 11, with experimental details provided in Example II.

### Second Generation Chimeras (Second Embodiment for Generation of Chimera):

1. Using the crystal structure of the groove MHC-I allele bound to peptide P in conformation C, Applicants select all residues in the groove MHC-I allele's sequence that have at least one heavy atom within 3 Å of a peptide heavy atom. Applicants term this set of residues first neighbors. Similarly, Applicants select all the residues in the groove MHC-I allele's sequence that have at least one heavy atom within 3 Å of a first neighbor heavy atom and term those residues second neighbors.

2. Applicants, aided by computer software tools, reviews all polymorphic first neighbors in the groove MHC-I structural model and introduces a sub-set of substitutions in the sequence of the base MHC-I allele to match the equivalent positions from the sequence of the groove MHC-I allele. This process defines the sequence of an MHC-Iₘ₁ mutant.

3. Applicants review all polymorphic second neighbors in the groove MHC-I structural model taking into account the mutations introduced to MHC-Iₘ₁ mutant and then introduces a sub-set of substitutions in the polymorphic second neighbors to the sequence of MHC-Iₘ₁ mutant to match the equivalent positions from the sequence of the groove MHC-I allele. This process defines the sequence of an MHC-Iₘ₂ mutant.

4. Applicants thread the sequence of the MHC-Iₘ₂ mutant onto the groove MHC-I and use a computer software to assess the remaining polymorphic positions that were not mutated to match the equivalent positions from the sequence of the groove MHC-I allele for any clashes with the mutations made in steps 2 or 3. Structure (See step IV in Example I, implementation and software).

5. In the MHC-Iₘ₂ mutant sequence, Applicants mutate any unmutated polymorphic positions that cause clashes with mutations introduced in steps 2 and 3 to match the equivalent positions from the sequence of the groove MHC-I allele. Applicants term the generated sequence a 2^{nd}-generation chimera MHC-I.

6. Using a computer software, Applicants thread the chimera MHC-I sequence onto the groove MHC-I allele structure, and refine the generated structure to find a local free energy minimum, which corresponds to the stable folded state of the protein. Applicants use the model of the chimeric structure to evaluate its stability and peptide binding free energy, and compare it with those derived from the groove MHC X-ray structure (See step IV in Example I, implementation and software).

### Triple Chimeras (Third Embodiment for Generation of Chimera):

In addition, Applicants propose the following protocol for generating synthetic MHC-I alleles which can bind to desired peptides where no known examples of naturally occurring epitopes and peptide: MHC-I structures are available in the Immune Epitope Database (Vita et al., 2019) and Protein Data Bank (PDB), respectively. This is done by introducing specific amino acid substitutions in the peptide binding groove of a base MHC allele, to match residues from the grooves of two relevant MHC-I alleles (anchor2 and anchor9) with known peptide binding specificities at anchor positions P2 and P9, respectively, and established X-ray structures, as follows:

1. Using the crystal structure of the anchor2 MHC-I allele bound to peptide P in conformation C, Applicants select all residues in the groove MHC-I allele's sequence that have at least one sidechain heavy atom within 3 Å of peptide residue P2. Applicants term this set of residues anchor2 first neighbors. Similarly, Applicants select all the residues in the groove MHC-I allele's sequence that have at least one heavy atom within 3 Å of an anchor2 first neighbor heavy atom and term those residues anchor2 second neighbors.

2. Applicants review all polymorphic anchor2 first neighbors in the anchor2 groove MHC-I structural model and introduces a sub-set of substitutions in the sequence of the base MHC-I allele according to the equivalent positions in the sequence of the anchor2 groove MHC-I allele. This process defines the sequence of an MHC-Iₘ₁ mutant.

3. Applicants review the MHC-Iₘ₁ mutant and all polymorphic second neighbors in the groove MHC-I structural model taking into account the mutations introduced to MHC-Iₘ₁ mutant and then introduces a sub-set of substitutions in the polymorphic second neighbors to the sequence of MHC-Iₘ₁ mutant to match the equivalent positions from the sequence of the anchor2 groove MHC-I allele. This process defines the sequence of an MHC-Iₘ₂ mutant.

4. Steps 1-3 are repeated with the crystal structure of the anchor9 MHC-I allele to generate a series of amino acid substitutions at the vicinity of the P9 peptide anchor.

5. The mutations identified using the structures of the anchor2 MHC-Iₘ₂ and anchor9 MHC-Iₘ₂ are then introduced into the sequence of the base allele. If a single position is mutated to two different residues in anchor2 MHC-Iₘ₂ and anchor9 MHC-Iₘ₂, the mutation is assessed by Applicants who picks either the anchor2 or anchor9 MHC-Iₘ₂ mutation to match in the consensus sequence. Applicants term this consensus sequence MHC-Iₘ₃.

6. Using a computer software, Applicants thread the consensus sequence MHC-Iₘ on both anchor2 and anchor9 groove MHC-I allele structures, and the sequence of the desired peptide through the peptide presented in the respective structures. Applicants then use a computer software to assess the remaining polymorphic positions that were not mutated to match the equivalent positions from the sequence of the groove MHC-I allele for any clashes with the mutations made in steps 2-5.

7. In the MHC-Iₘ₃ mutant sequence, Applicants mutate any unmutated polymorphic positions that cause clashes with mutations introduced in steps 2-5 to match the equivalent positions from either anchor2 or anchor9 groove MHC-I allele to minimize the clash score. Applicants term this final sequence MHC-Iₘ₃.

8. Using a computer software, Applicants thread the final sequence MHC-Iₘ on both anchor2 and anchor9 groove MHC-I allele structures, and the sequence of the desired peptide through the peptide presented in the respective structures. Applicants then refine both generated structures to find a local free energy minimum, which corresponds to the stable folded state of the protein. Applicants models the chimeric structure to evaluate its stability and peptide binding free energy. The lowest-energy model is then selected as a plausible model of the triple chimera MHC-I structure.

The invention involves a computer-assisted method for identifying or designing potential compounds to fit within or bind to the chimeras of the present invention or a functional portion thereof or vice versa (a computer-assisted method for identifying or designing potential chimeras or a functional portion thereof for binding to desired compounds) or a computer-assisted method for identifying or designing potential chimeras (e.g., with regard to predicting areas of the chimera to be able to be manipulated), said method comprising using a computer system, e.g., a programmed computer comprising a processor, a data storage system, an input device, and an output device, the steps of: (a) inputting into the programmed computer through said input device data comprising the three-dimensional co-ordinates of a subset of the atoms from or pertaining to chimera crystal structure, thereby generating a data set; (b) comparing, using said processor, said data set to a computer database of structures stored in said computer data storage system, e.g., structures of compounds that bind or putatively bind or that are desired to bind to a chimera of the present invention or as to a chimera structure; (c) selecting from said database, using computer methods, structure(s)—e.g., chimeras that may bind to desired structures, desired structures that may bind to certain chimeras, portions of the chimeras that may be manipulated; (d) constructing, using computer methods, a model of the selected structure(s); and (e) outputting to said output device the selected structure(s); and optionally synthesizing one or more of the selected structure(s); and further optionally testing said synthesized selected structure(s) as or in a chimera.

The testing can comprise analyzing the chimera resulting from said synthesized selected structure(s), e.g., with respect to binding, or performing a desired function.

The computer-assisted methods described herein may be performed iteratively, so as to improve the computer implementation by performance of said methods. For instance, once the selected structure(s) are output via said output device, one or more of the selected structure(s) may be synthesized and tested. The results of the synthesizing and testing may then be re-input into the data set(s) or a memory device of the computer system or stored data thereon (e.g., learned data set(s) for making comparisons and accepting or rejecting structures) for being employed in a further iteration of the computer-assisted methods. This process may then be repeated until a desired or acceptable output is obtained.

The output in the foregoing methods can comprise data transmission, e.g., transmission of information via telecommunication, telephone, video conference, mass communication, e.g., presentation such as a computer presentation (e.g., POWERPOINT), internet, email, documentary communication such as a computer program (e.g., WORD) document and the like. Accordingly, the invention also comprehends computer readable media structural data, said data defining the three dimensional structure of a chimera of the present invention or at least one sub-domain thereof, or structure factor data for a chimera. The computer readable media can also contain any data of the foregoing methods.

The invention further comprehends methods a computer system for generating or performing rational design as in the foregoing methods, said data defining a three dimensional structure of any of the chimeras of the claimed invention or at least one sub-domain thereof, or structure factor data for a chimera.

The invention further comprehends a method of doing business comprising providing to a user the computer system or the media or the three dimensional structure of a chimera of the present invention or at least one sub-domain thereof, or structure factor data for a chimera of the present invention, or the herein computer media or a herein data transmission.

A “binding site” or an “active site” comprises or consists essentially of a site (such as an atom, a functional group of an amino acid residue or a plurality of such atoms and/or groups) in a binding cavity or region, which may bind to a compound such as a nucleic acid molecule, which is/are involved in binding.

By “fitting”, is meant determining by automatic, or semi-automatic means, interactions between one or more atoms of a candidate molecule and at least one atom of a structure of the invention, and calculating the extent to which such interactions are stable. Interactions include attraction and repulsion, brought about by charge, steric considerations and the like. Various computer-based methods for fitting are described further

By a “computer system”, is meant the hardware means, software means and data storage means used to analyze atomic coordinate data. The minimum hardware means of the computer-based systems of the present invention typically comprises a central processing unit (CPU), input means, output means and data storage means. Desirably a display or monitor is provided to visualize structure data. The data storage means may be RAM or means for accessing computer readable media of the invention. Examples of such systems are computer and tablet devices running Unix, Windows or Apple operating systems.

By “computer readable media”, is meant any medium or media, which can be read and accessed directly or indirectly by a computer e.g. so that the media is suitable for use in the above-mentioned computer system. Such media include, but are not limited to: magnetic storage media such as floppy discs, hard disc storage medium and magnetic tape; optical storage media such as optical discs or CD-ROM; electrical storage media such as RAM and ROM; thumb drive devices; cloud storage devices and hybrids of these categories such as magnetic/optical storage media.

Regarding FIG. 6, one may perform the steps of the first embodiment for generation of chimera, or the second embodiment for generation of chimera, or third embodiment for generation of chimera, each involving computer implementation, storage of data on a memory device, and the data including data set(s) for making comparisons and accepting or rejecting structures, and with each involving synthesis and expression, either in E. coli or another suitable vector or cell, including as herein further discussed: Each step involves computer implementation that includes the use of methods, algorithms, and/or scripts that are known in the art.

A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety.

After performing those steps and preparing the chimera, with reference to FIG. 6, left hand side, the chimera is incubated with a sample containing a peptide of interest (binding of peptide to chimera promotes folding of the peptide/MHC/b2m protein complex). And there is detection of folding via antibody-based analysis (e.g., ELISA, e.g. W6/32), and affinity-based purification of pMHC proteins and elution of bound peptides followed by proteomics analysis (e.g. by mass spectrometry). The information from these further steps in an embodiment is inputted into the data set(s) or the memory device or stored data thereon for being employed in further computer implementation of the first embodiment for generation of chimera, or the second embodiment for generation of chimera, or third embodiment for generation of chimera.

The information from performing the first embodiment for generation of chimera, or the second embodiment for generation of chimera, or third embodiment for generation of chimera and the left hand side steps, can be used in selecting chimera for performing the methods of the right hand side steps of FIG. 6 as well as the steps of the middle of FIG. 6.

With regard to each of the embodiments of the middle and right hand side of FIG. 6, either the first embodiment for generation of chimera, or the second embodiment for generation of chimera, or third embodiment for generation of chimera have been performed and the information therefrom is being used for synthesis of the chimera, or nucleic acid molecule(s) encoding the chimera, or the process of the left hand side of FIG. 6 has been performed and information therefrom has been inputted and the chimera to be employed in the methods of the middle and right hand side of FIG. 6 are from having at least once first performed the left hand side of FIG. 6 and inputted information therefrom into the data set(s) or memory device or the computer implementation includes data from having at least performed the left hand side of FIG. 6.

In any such embodiment of the middle or right hand side of FIG. 6: Starting with the middle of FIG. 6, T-cell(s) are contacted with the chimera to obtain modified T-cell(s) comprising T-cell(s) identified by recognition of a chimera peptide: MHC complex. This optionally further comprises expanding the T-cell(s) into a modified T-cell population, wherein the T-cell(s) used in the contacting can be isolated from a patient or subject, and optionally altered therefrom by having or introducing desired coding nucleic acid molecule(s) and/or by expressing desired product(s), optionally said introducing through a lentivirus system. The T cell(s) used in the contacting can having a particular TCR expressed, e.g., by genetic modification, or naturally, or a can be a CAR-T, e.g., a T cell altered to express certain antigen receptor(s), or the T-cell can be patient-derived such as a Tumor Infiltrating Lymphocyte. The chimera can be provided into the system as a selection marker, e.g., in a bead or tetramerized form. The T-cell can thus be used for antigen targeting, e.g., for therapeutic, clinical or research purposes, and data therefrom can be inputted into the memory device or the data set(s) to enhance further computer implementation of the first embodiment for generation of chimera, or the second embodiment for generation of chimera, or third embodiment for generation of chimera.

T cells are one of the cell populations playing major roles in the immune system as a biodefence system against various pathogens. Such T cells are roughly classified into CD4 positive helper T cells and CD8 positive cytotoxic T cells, where the former relates to the promotion of immune response and the latter relates to the exclusion of virus-infected cells and tumor cells. Helper T cells are further classified into Type I helper T cells for promoting cellular immunity and Type II helper T cells for promoting humoral immunity. These T cells with such diversified properties have a function of excluding pathogens and gaining infection resistance under a well-balanced immune response.

In preparing the pharmaceutical compositions containing the T-cell population of the present invention, a desired dosage form can be selected depending on their therapeutic purposes, administration routes, or the like. It is desirable that the T-cell population contained in the pharmaceutical compositions should be administered alive and the T-cell population is usually used intact after being suspended in a liquid or embedded with a gel along with an appropriate additive, or optionally encapsulated in an appropriate microcapsule or liposome before parenterally administered through injection or the like. It can be also used in such a manner of freezing to preserve the T-cell population suspended in a medium or physiological saline containing dimethylsulfoxide or glycerine, and thawing the freezed product prior to use.

The middle of FIG. 6 can be to create in the T cell a tolerance breaking interaction (e.g., use of the invention to break immune tolerance).

The left side of FIG. 6 provides also a means for generating novel T cell populations, especially T cell populations that can target an antigen of interest.

In the right hand side of FIG. 6, a dendritic cell is genetically modified e.g., via a CRISPR system such as a CRISPR-Cas9 system and the coding for the chimeric is inserted into the dendritic cell, whereby the dendritic cell is expanded into a cell population and can provide T cells e.g., T cells that target a certain antigen such as an antigen on a cell of a particular type of cancer, such as a solid cancer. Data from the interaction of the resulting T cells with cancer cells, such as solid cancer cells, can be inputted into the memory device or the data set(s) to enhance further computer implementation of the first embodiment for generation of chimera, or the second embodiment for generation of chimera, or third embodiment for generation of chimera.

In general, the CRISPR-Cas or CRISPR system refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell. In some embodiments, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used. In some embodiments it may be preferred in a CRISPR complex that the tracr sequence has one or more hairpins and is 30 or more nucleotides in length, 40 or more nucleotides in length, or 50 or more nucleotides in length; the guide sequence is between 10 to 30 nucleotides in length, the CRISPR/Cas enzyme is a Type II Cas9 enzyme.

The left side of FIG. 6 also provides a means for generating novel T cell populations, especially T cell populations that can target an antigen of interest such as an antigen of interest on a cancer cell and thus can provide a medicament, especially if the T cell is that from a patient or subject who has the cancer.

The term “cancer” according to the invention comprises leukemias, seminomas, melanomas, teratomas, lymphomas, neuroblastomas, glioblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer and lung cancer and the metastases thereof. Examples thereof are lung carcinomas, mamma carcinomas, prostate carcinomas, colon carcinomas, renal cell carcinomas, cervical carcinomas, or metastases of the cancer types or tumors described above. The term cancer according to the invention also comprises cancer metastases and relapse of cancer.

The therapeutically active agents, vaccines and compositions described herein may be administered via any conventional route, including by injection or infusion. The administration may be carried out, for example, orally, intravenously, intraperitoneally, intramuscularly, subcutaneously or transdermally. In one embodiment, administration is carried out intranodally such as by injection into a lymph node. Other forms of administration envision the in vitro transfection of antigen presenting cells such as dendritic cells with nucleic acids described herein followed by administration of the antigen presenting cells.

The agents described herein are administered in effective amounts. An “effective amount” refers to the amount which achieves a desired reaction or a desired effect alone or together with further doses. In the case of treatment of a particular disease or of a particular condition, the desired reaction preferably relates to inhibition of the course of the disease. This comprises slowing down the progress of the disease and, in particular, interrupting or reversing the progress of the disease. The desired reaction in a treatment of a disease or of a condition may also be delay of the onset or a prevention of the onset of said disease or said condition.

An effective amount of an agent described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the agents described herein may depend on various of such parameters. In the case that a reaction in a patient is insufficient with an initial dose, higher doses (or effectively higher doses achieved by a different, more localized route of administration) may be used.

The pharmaceutical compositions of the invention are preferably sterile and contain an effective amount of the therapeutically active substance to generate the desired reaction or the desired effect.

The pharmaceutical compositions of the invention are generally administered in pharmaceutically compatible amounts and in pharmaceutically compatible preparation. The term “pharmaceutically compatible” refers to a nontoxic material which does not interact with the action of the active component of the pharmaceutical composition. Preparations of this kind may usually contain salts, buffer substances, preservatives, carriers, supplementing immunity-enhancing substances such as adjuvants, e.g. CpG oligonucleotides, cytokines, chemokines, saponin, GM-CSF and/or RNA and, where appropriate, other therapeutically active compounds. When used in medicine, the salts should be pharmaceutically compatible. However, salts which are not pharmaceutically compatible may be used for preparing pharmaceutically compatible salts and are included in the invention. Pharmacologically and pharmaceutically compatible salts of this kind comprise in a non-limiting way those prepared from the following acids: hydrochloric, hydrobromic, sulfuric, nitric, phosphoric, maleic, acetic, salicylic, citric, formic, malonic, succinic acids, and the like. Pharmaceutically compatible salts may also be prepared as alkali metal salts or alkaline earth metal salts, such as sodium salts, potassium salts or calcium salts.

A pharmaceutical composition of the invention may comprise a pharmaceutically compatible carrier. The term “carrier” refers to an organic or inorganic component, of a natural or synthetic nature, in which the active component is combined in order to facilitate application. According to the invention, the term “pharmaceutically compatible carrier” includes one or more compatible solid or liquid fillers, diluents or encapsulating substances, which are suitable for administration to a patient. The components of the pharmaceutical composition of the invention are usually such that no interaction occurs which substantially impairs the desired pharmaceutical efficacy.

The pharmaceutical compositions of the invention may contain suitable buffer substances such as acetic acid in a salt, citric acid in a salt, boric acid in a salt and phosphoric acid in a salt.

The pharmaceutical compositions may, where appropriate, also contain suitable preservatives such as benzalkonium chloride, chlorobutanol, paraben and thimerosal.

The pharmaceutical compositions are usually provided in a uniform dosage form and may be prepared in a manner known per se. Pharmaceutical compositions of the invention may be in the form of capsules, tablets, lozenges, solutions, suspensions, syrups, elixirs or in the form of an emulsion, for example.

Compositions suitable for parenteral administration usually comprise a sterile aqueous or nonaqueous preparation of the active compound, which is preferably isotonic to the blood of the recipient. Examples of compatible carriers and solvents are Ringer solution and isotonic sodium chloride solution. In addition, usually sterile, fixed oils are used as solution or suspension medium.

The Figures and Appendices herewith (e.g., FIGS. 11 and 16-19, and Appendices 1-3) further confirm that the invention can be utilized in the design of peptide-centric therapeutics. The chimera of the invention can be used to recognize peptide-MHC moieties, irrespective of the peptide, e.g., that the chimera can be used to recognize three dimensional structures, and hence can be used to screen for reagents that can touch, contact or interact with residues and form a complex. The tetramerized form is especially useful. Thus, products from any means of producing mass quantities of expression products that may be different, e.g., yeast display systems, phage display systems or directed evolution, can be screened using the chimera of the invention to determine alignment of three-dimensional structure with pMHC structure. Accordingly, the invention comprehends screening expression products, such as those from yeast display systems, phage display systems or directed evolution, by contacting expression products with the pMHC structure, or by aligning data as to the three dimensional structure of the expression products with the crystal structure(s) provided herewith, for instance using computer implementation to compare said three dimensional structure of the expression products with the crystal structure(s) provided herewith. Thus, the disclosure herein of the FIGS. and Appendices, e.g., FIGS. 11 and 16-19, and Appendices 1-3, can be applied in the techniques herein discussed as to FIG. 6.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1 Implementation & Software Tools

### Preprocessing:

I. All MHC groove structures were preprocessed using pdb-tools. Hetero atoms, the β chain, and the α₃ domain of the heavy chain were removed. Structures were renumbered such that the first residue in the structure has residue ID one.

```
  $python ./pdbtools/pdb_delres.py −181: 4qrt.pdb > 4qrt_trim.pdb
  $python ./pdb-tools-master/pdbtools/pdb_delhetatm.py 4qrt_trim.pdb >
4qrt_trim_noHet.pdb
  $python ./pdb-tools-master/pdbtools/pdb_delchain.py -B 4qrt_trim_noHet.pdb >
4qrt_trim_noHet_noB.pdb
```

Generating visual aids for the design process of 1^{st}-generation chimeras:

II. The script chimera_generator.py (to be attached in the supplements) was executed. The script takes as input the processed PDB structure of the groove MHC and a sequence of the base MHC which can be directly pasted into the command line or automatically fetched from a provided base PDB.

./chimera_generator.py
PDB ID of groove HLA (ex. 3rl1.pdb): 4qrt_trim_noHet_noB.pdb
PDB ID or sequence of base HLA (ex. 5hhn.pdb or PWEASRSAEAP...):
5HHN.pdb

The script performs a sequence alignment of the groove MHC sequence with the base MHC sequence to identify all polymorphic sites.

The groove MHC structure is used to identify all polymorphic residues that have a sidechain heavy atom within 5 Å of any peptide heavy atoms.

The algorithm returns a list of all polymorphic sites, the identity of the residue in the base MHC and the groove MHC at the polymorphic sites, and the sequence of the first generation chimeric MHC as described in methods (steps 2-4). For second generation chimeras and triple chimeras, only a select subset of the polymorphic positions reported by chimera generator are selected for introduction into the base allele sequence as described in methods.

III. The chimeric MHC sequence is threaded on the groove PDB structure using Rosetta's partial_thread.

```
  Rosetta/main/source/bin/partial_thread.linuxgccrelease \ -database Rosetta/main/database \
  -in:file:fasta chimera.fasta \
  -in:file:alignment 5hhn_on_4qrt_trim_noHet_noB.aln \ -in:file:template_pdb
4qrt_trim_noHet_noB.pdb \ -ignore_unrecognized_res
```

IV. The threaded structure is refined using Rosetta's fast relax with constraints applied to the peptide backbone and side chain degrees of freedom to restrict the movement of all peptide degrees of freedom. Then, the peptide: MHC binding energy is calculated using Rosetta's interface_energy protocol.

Rosetta scripts file:
#!/Users/Sani/opt/anaconda3/bin/python
“““change the first line to your python location”””
“““this code aligns a set previously generated backbone conformation of peptide
with a pMHC complex then saves the new files with a ‘c’ suffix”””

V. The generated structure is analyzed using MolProbity

(http://molprobity.biochem.duke.edu), and a clash score is calculated which reports on the overall quality of the modeled chimera structure.

VI. Steps 3-6 are repeated with the base MHC sequence and the groove MHC sequence for comparison. If the binding energy, total energy, and clash score of the chimeric structure are significantly lower than that of the base structure following threading and relaxing, the design is cleared for experimental verification.

### Example 2: Experimental Validation of Designs

### Protein Expression, Refolding and Purification

Synthetic, codon-optimized genes encoding the luminal domain of the chimeric class I MHC heavy chains HLA-A*11: 01/A*02:01, HLA-B*08:01/A*02:01, HLA-C*07: 02/A*02:01 and the human β₂m (hβ₂m, light chain) cloned into pET-22b vector were purchased from GenScript and transformed into Escherichia coli BL21 (DE3) (Novagen). Proteins were expressed in Luria-Broth and inclusion bodies were isolated as previously described (Li et al., 1998). For the in vitro refolding, a 200 mg mixture of 1:3 molar ratio of heavy chain: light chain was slowly diluted over 24 hours into 1 L of refolding buffer (0.4 M Arginine-HCl, 2 mM EDTA, 4.9 mM reduced L-glutathione, 0.57 mM oxidized L-glutathione, 100 mM Tris pH 8.0) containing 10 mg of the desired peptide to be refolded with the chimeric MHC-I at 4° C. while stirring. Refolding proceeded for four days at 4° C. without stirring (Garboczi et al., 1992). Proteins were dialyzed in 10 L of dialysis buffer (150 mM NaCl, 25 mM Tris pH 8.0) using 3500 MWCO 54 mm (Spectra/Por #132725) membranes, for 12 h with spinning at 4° C. Purification of pMHC-I complexes was performed by Size Exclusion Chromatography (SEC) using a HiLoad 16/100 Superdex 75 pg column at 1 mL/min using SEC buffer (150 mM NaCl, 25 mM Tris pH 8.0). Protein concentration was determined using NanoDrop A280 measurements and the extinction coefficients calculated using the ExPASy ProtParam Tool.

### Differential Scanning Fluorimetry

DSF experiments were performed on an Applied Biosystems 7900HT Fast Real-Time PCR system using MicroAmp Optical 384-well plates with 20 μL total volume containing final concentrations of 7 μM chimeric MHC-I protein in buffer of 150 mM NaCl, 20 mM sodium phosphate pH 7.2 and 10×SYPRO orange dye (ThermoFisher). Samples were centrifuged at 13,000 rpm for 10 min prior assay to remove precipitates. The temperature was increased at a scan rate of 1° C./min between 25° C. and 95° C. and fluorescence was monitored in the ROX channel (Hellman et al., 2016). Experiments were conducted in triplicate and data analysis and fitting was performed using GraphPad Prism v7.

### Example 3: Further Validation of Designs

Reference is made to FIGS. 16, 17 18 and 19. FIG. 16 shows that the pMHC structure presents distinct surfaces for interactions with T-Cell Receptors (TCRs) and peptides. FIG. 17 shows A02 engineered A11 groove specificity recapitulates (or repeats or maintains) the original peptide conformation (three-dimensional conformation), demonstrating that chimera of the invention have fidelity as to three-dimensional conformation. FIG. 18 shows A02 engineered B08 groove specificity recapitulates (or repeats or maintains) the original peptide conformation (three-dimensional conformation), demonstrating that chimera of the invention have fidelity as to three-dimensional conformation. FIG. 19 shows that tetramerized A02/B08 chimeras can probe the HLA allele dependence of TCR interactions, demonstrating the utility of the present invention for screening for reagents that interact with residues of the complex in comparison with residues from the peptide (such that in peptide-centric therapeutics, e.g., products from any sort of system, e.g., phage display, yeast display, directed evolution, can be screened using tetramerized chimeras of the invention as they recognize peptide-MHC moiety irrespective of the peptide).

More in particular, FIG. 16 provides that for each position, X, in the HLA sequence, an amino acid consensus score, TCR Contact frequency, and Peptide Contact frequency was calculated. The consensus score at a position is the percent of HLA sequences in the HLA database that have the most common amino acid at said position. The TCR contact frequency for position X is the percent of pHLA-TCR structures in which position X is within 4A of TCR residue. Likewise, The Peptide contact frequency for position X is the percent of pHLA structures in which position X is within 4A of a peptide residue.

FIG. 17 presents the solved crystal structure of A*11:01-A*02:01/HIV-1 RT chimera, solved to 2.0 Å. The peptide P1/P2, P5, and P9 residues are highlighted in the A*11:01-A*02:01/HIV-1 RT chimera structure, particularly the P9 positions. The HIV-1 RT peptide for the chimera and A*11:01 WT structures are overlaid to demonstrate identical conformations.

FIG. 18 presents the solved crystal structure of B*08:01-A*02:01/CMV chimera, solved to 2.7 Å. The peptide P1/P2, P5, and P9 residues are highlighted in the B*08:01-A*02:01/CMV chimera structure, particularly the P5 positions. The CMV peptide for the chimera and A*11:01 WT structures are overlaid to demonstrate identical conformations.

FIG. 19 demonstrates that tetramerized A*02:01 complexes displaying NY-ESO-1 are recognized by the TCR 1G4 and can be stained using primary T cells transfected with 1G4 TCR using flow cytometry, while a single alanine substitution of W5 in the NY-ESO-1 peptide disrupts binding of 1G4 and does not stain using flow cytometry. Using tetramerized A*02:01-B*08:01/NY-ESO-1 chimera, we demonstrate the 1G4 TCR very weakly binds as analyzed by flow cytometry, suggesting the TCR interaction surfaces are disrupted by the new B*08:01 base.

### Example 4: Decoupling Peptide Binding from T Cell Receptor Recognition with Engineered Chimeric MHC-I Molecules

Major Histocompatibility Complex class I (MHC-I) molecules display self, viral or aberrant epitopic peptides to T cell receptors (TCRs), which employ interactions between complementarity-determining regions with both peptide and MHC-I heavy chain ‘framework’ residues to recognize specific Human Leucocyte Antigens (HLAs). The highly polymorphic nature of the HLA peptide-binding groove suggests a malleability of interactions within a common structural scaffold. Here, using structural data from peptide: MHC-I and pMHC: TCR structures, Applicants first identify residues important for peptide and/or TCR binding. Applicants then outline a fixed-backbone computational design approach for engineering synthetic molecules that combine peptide binding and TCR recognition surfaces from existing HLA allotypes. X-ray crystallography demonstrates that chimeric molecules bridging divergent HLA alleles can bind selected peptide antigens in a specified backbone conformation. Finally, in vitro tetramer staining and biophysical binding experiments using chimeric pMHC-I molecules presenting established antigens further demonstrate the requirement of TCR recognition on interactions with HLA framework residues, as opposed to interactions with peptide-centric Chimeric Antigen Receptors (CARs). Applicants' results underscore a novel, structure-guided platform for developing synthetic HLA molecules with desired properties as screening probes for peptide-centric interactions with TCRs and other therapeutic modalities.

The class I proteins of the Major Histocompatibility Complex (MHC-I) present epitopic peptide antigens on the cell surface, thereby enabling immune surveillance of the intracellular proteome by CD8+ T cells and Natural Killer cells (1-5). Under physiological conditions, peptide: MHC (pMHC-I) molecules are assembled in the endoplasmic reticulum (ER) and are trafficked to the cell surface to present a pool of millions of different peptides derived from either host (self-peptides) or aberrant proteins, including viral factors and dysregulated oncoproteins (non-self-peptides) (2). The human MHC-I molecules, referred to as Human Leukocyte Antigens (HLAs), are among the most polymorphic genes with over 35,000 different allotypes reported in the human genome and are classified into the HLA-A, -B, and -C subfamilies (6-10). Several studies have proposed that the vast HLA diversity and extended peptide binding repertoire was driven by evolutionary pressures to adapt in pathogen-rich environments (11-14). Nonetheless, HLAs are structurally conserved with a variable heavy chain, an invariant light chain (β₂-microglobulin, β₂m), and a bound peptide typically ranging between 8-15 amino acids in length (15-18). The heavy chain is comprised of three domains, the α₁ and α₂ helices define the peptide binding groove in the MHC-I structure, while α₃ stabilizes the molecule by creating an extensive binding interface with β2m. The peptide-binding groove consists of several adjacent ‘pockets’ referred to as A-F, and polymorphisms within the groove govern the respective antigen repertoire of different HLA allotypes, and induce specific peptide conformations (17, 19). While in most HLA allotypes, such as the common HLA-A*02:01 allele, the B- and F-pockets are the primary sites of stabilizing interactions with two specific peptide anchor residues at positions 2 (P2) and 9 (P9), respectively, several allotypes exhibit different anchor residues (20, 21). These variations across different HLA allotypes enable immune surveillance of diverse peptide repertoires at the population level, thus ensuring species adaptability to emerging pathogens (22).

The ability of T cells to recognize epitopic peptides in the context of specific MHC molecules is known as MHC restriction, and two hypotheses have been proposed to explain this phenomenon. The clonal selection theory poses that only TCRs binding specific MHCs will survive thymic selection (23), whereas the germline hypothesis supports that TCRs co-evolved for inherent reactivity to their MHC counterparts (24). However, experimental data for and against both models suggest that they are not mutually exclusive, and can be interpreted by a combined hypothesis (25). Cell-mediated adaptive immune responses depend upon recognition of specific pMHC-I proteins by T cell receptors present in a polyclonal repertoire encompassing 1×10⁸ distinct antigen specificities, leading to stimulation and clonal expansion (26, 27). The association between pMHC-I molecules and TCRs is highly dependent upon interactions with polymorphic residues on the α₁ and α₂ helices, as well as with exposed peptide residues. These interactions are mediated by six complementarity-determining regions (CDRs) within the variable domains of the TCR-α and -β chains, which adopt a classical diagonal orientation (25, 28-31). T cells are required to respond to a large number of different epitopic peptides, therefore TCR interactions with their pHLA antigens are characterized by a high degree of cross-reactivity, and inherently low affinity interactions to mitigate the risk of autoimmune responses. A recent study has employed targeted mutagenesis of conserved residues on the α₁ and α₂ helices which mediate key germline interactions with TCRs, to enhance recognition by alloreactive T cells while preserving the presentation of peptide antigens in a conserved conformation (32), as a means to break tolerance for specific self-antigens with possible applications in cancer therapy (33). This work provides a rationale for the design of synthetic molecules bridging TCR recognition surfaces with peptide-binding specificities from multiple HLA allotypes as a potential platform for eliciting CD8+ responses against specific tumor-associated antigens. More recently, the advent of peptide-centric, antibody based pMHC engagers as targeting modalities for Chimeric Antigen Receptor (CAR) T cell therapy highlight one additional application of synthetic HLA molecules as probes to screen for and verify allotype-independent recognition of specific antigens with the potential to treat a broader cohort of patients (34). The wide range of peptide-binding specificities covered by the known HLA allotypes is attained through specific combinations of the 33 polymorphic residues which mediate peptide binding (6, 35), suggesting that the peptide-binding groove provides a highly malleable structural scaffold for protein engineering applications aiming to expand naturally occurring T cell repertoires, or to design novel HLA-targeted therapeutics.

Here, Applicants perform an extensive analysis of existing pMHC-I and pMHC-TCR structures to identify key residues that form contacts with peptides and TCRs, respectively. Applicants then outline a systematic, fixed-backbone approach for engineering synthetic MHC-I molecules with desired peptide binding and TCR interface properties. Using the HLA-A*02:01, B*08:01 and B*35:01 alleles as structural scaffolds Applicants generate stable, properly conformed molecules encompassing the peptide-binding specificities of divergent allotypes, including HLA-A*11:01, A*24:02, B*08:01, A*02:01 and C*07:02. Applicants demonstrate that the designed molecules form stable complexes with peptides specific for the desired HLA groove, and adopt an identical conformation compared to their parental, wild-type pMHC-I complexes. Finally, Applicants provide direct evidence that engineered chimeric HLAs presenting disease-related epitopes disrupt interactions with known TCRs but not with peptide-centric CARS, highlighting the importance of HLA framework residues in TCR recognition. Applicants' results underscore a use of chimeric HLAs as screening probes to identify and expand TCR or CAR specificities for distinct peptide antigens, with a minimal reliance on interactions with HLA framework residues. Conversely, in analogy to altered peptide ligands (36, 37), chimeric HLAs provide a rational approach to manipulate interactions between established peptide: HLA antigens and their TCR repertoires in applications aiming to overcome central and peripheral tolerance for eliciting cross-reactive T cell responses against specific self-antigens that are overexpressed in tumor cells, as supported by previous studies (33).

Chimeric MHC-I generation. Chimeric MHC-I molecules were designed using ‘CHaMeleon’, a fixed-backbone approach developed herein. The method requires the structure of an MHC-I allele that binds a desired peptide (groove or template allele), and the sequence of an MHC-I allele with different peptide repertoire and TCR contact surfaces of interest (base allele). The structure of the groove allele was preprocessed to optimize its compatibility with the Rosetta software (38). Only the α₁ and α₂ helices of the MHC-I heavy chain and the bound peptide were retained, while the conserved α₃ domain of the heavy chain, the light chain, and all cofactors were removed to reduce the computing time in the subsequent relax protocol. The residues in the structure were renumbered such that the first residue in the structure had residue ID one (FIG. 39A). The peptide binding groove of the template allele was defined as the set of residues within 5 Å of a peptide heavy atom on the processed structure using PyMOL (FIG. 39B). A sequence alignment between the groove MHC-I allele and the base MHC-I allele was performed using EMBOSS Needle pairwise sequence alignment (EMBL-EBI). Starting with the base allele sequence, the chimeric MHC-I sequence was created by substituting every residue in the peptide-binding groove of the base with the corresponding residue of the template allele. To assess the stability and binding affinities of the generated chimeric HLAs, Applicants created and evaluated the structures by threading the chimeric sequence through the preprocessed base allele structure using RosettaCM (FIG. 39C). The threaded structures were then relaxed using the score function ‘REF2015’ in Rosetta (FIGS. 39D-39F). Since Applicants were only interested in the structures that bound the target peptide in the same conformation as the groove allele, the peptide residues were fixed in place using ‘PreventRepackingRLT’. The ‘Fast_Relax Mover’ was used with 3 repeats of the relax protocol allowing both the side chains and backbone of the heavy chain to relax during the simulation. ‘InterfaceAnalyzerMover’ was then used to calculate the binding energy of the peptide to the chimeric MHC-I, after repacking them separately using the ‘pack_seperated’ option. The standard options were used to optimize computational cost while creating realistic relaxed structures (FIG. 39D). The options used in the command line were: ‘-nstruct 3’ to generate three relaxed structures and calculate total and binding energies in each of the triplicates, ‘-no_optH’ to prevent hydrogen placement optimization, ‘flip_HNQ’ to prevent flipping Histidine, Asparagine, and Glutamine, and ‘-use_input_sc’ to use the input rotamers as part of the rotamer set explored by the relax algorithm.

Combinatorial sampling of polymorphic groove residues. An exhaustive assessment of every possible chimeric molecule that could be generated was performed using Rosetta software (38). The sequence of the base allele was threaded through the preprocessed structure of the groove allele as described above (FIG. 39C). The threaded structure was then idealized and relaxed using Rosetta's applications with the default options. From three decoy output structures, Applicants used the most stable to introduce each set of mutations on the threaded structure of the base allele using Rosetta remodel. A blueprint file was generated for every possible combination of mutations in the polymorphic groove residues between the template and base alleles. For instance, for 9 polymorphic residues between two alleles within 5 Å of the peptide, 29=512 blueprint files would be generated and used in conjunction with Rosetta remodel to build 512 chimeric-MHC structures. The generated models were refined with a final relax step with a single decoy for each structure and were ranked based on the calculated peptide: MHC binding energy. For the top 2.5% of structures with the lowest energies, Applicants calculated the enrichment score for each polymorphic peptide binding groove position as the ratio of structures among the defined pool, in which a substitution from base to template allele residue was introduced.

Peptide sequence logo generation. The peptide binding profile of the designed chimeric HLAs was predicted using an in-house method based on NetMHCpan4.0 (39). Briefly, a list of all the experimentally measured peptide epitopes for the MHC class I alleles were extracted from IEDB (7) and were used to predict binding by the chimeric sequences using NetMHCpan4.0. The final sequence logos were generated using Seq2logo (40).

Recombinant protein expression, refolding, and purification. Plasmid DNA encoding the luminal domain of HLA-A*02:01 and A*24:02 heavy chains, and human β2m (β2m, light chain) were provided by Dale Long of the NIH Tetramer Core Facility. DNA encoding the HLA-A*11:01-A*02:01, A*11:01-A*02:016M, B*08:01-A*02:01, C*07:02-A*02:01, A*02:01-B*08:01, and A*24:02-B*35:01 chimeric constructs (FIG. 20) was cloned into pET-22b (+) vector using NdeI/BamHI restriction sites (Genscript). For tetramer staining and binding assays, proteins were tagged with the BirA substrate peptide (BSP, LHHILDAQKMVWNHR). The NYE-S1 TCR-α and -β chains were cloned into pET-22b (+) vector with NdeI/BamHI restriction sites (Genscript). DNA plasmids were transformed into Escherichia coli BL21 (DE3) (New England Biolabs). Proteins were expressed in Luria Broth and inclusion bodies were solubilized using guanidine hydrochloride as previously described (41). pMHC-I complexes were generated by in vitro refolding as 200 mg mixtures of heavy chain: light chain at a 1:3 molar ratio and 10 mg of peptide in 1 L of refolding buffer (0.4 M L-Arginine-HCl, 2 mM EDTA, 4.9 mM reduced L-Glutathione, 0.57 mM oxidized L-Glutathione, 100 mM Tris pH 8.0) at 4° C. MHC-I molecules refolded with photolabile peptides were protected from light with aluminum foil. Refolding proceeded for 4 days and the pMHC-I complexes were purified by size-exclusion chromatography (SEC) using a HiLoad 16/600 Superdex 75 pg column at 1 mL/min with 150 mM NaCl, 25 mM Tris buffer, pH 8.0. The luminal domain of the TCR NYE-S1 α/β complex was expressed and purified as previously described (30). The 10LH scFv protein was provided by Myrio Therapeutics (Australia). Protein concentrations were determined using A280 measurements on Nanodrop with extinction coefficients estimated by ExPASy ProtParam tool (42).

Peptides. A full list of the peptides used in this study and their abbreviations is shown in FIG. 33. All peptide sequences are given as standard single-letter codes and were purchased from Genscript, NJ, USA, at >90% purity. The photolabile peptide used was purchased from Biopeptek Inc, PA, USA, using J as 3-amino-3-(2-nitrophenyl)-propionic acid (43). For the peptide solutions, lyophilized peptides were solubilized in distilled water and centrifuged at 14,000 rpm for 15 min. Concentrations were calculated using the respective absorbance and extinction coefficient at 205 nm wavelength.

Differential scanning fluorimetry. For DSF experiments, samples were prepared at a final concentration of 7 μM in PBS buffer (50 mM NaCl, 20 mM sodium phosphate pH 7.2) and mixed with 10×SYPRO Orange dye (ThermoFisher) to a final volume of 20 μL. Samples were then loaded into a MicroAmp Fast 384-well plate and ran in triplicates (n=3) on a QuantStudio™ 5 Real-Time PCR machine with excitation and emission wavelengths set to 470 nm and 569 nm, respectively. Temperature was incrementally increased at a rate of 1° C./min between 25° C. and 95° C. to measure the thermal stability of the proteins. Data analysis and fitting were performed in GraphPad Prism v9.

Peptide exchange. Peptide exchange mediated by UV-irradiation was performed by incubating 7 μM of HLA-B*08:01-A*02:01/FLRGRAXGL (SEQ ID NO: 16) with 70 μM of the desired peptide in PBS buffer (50 mM NaCl, 20 mM sodium phosphate pH 7.2) for 1 hour at room temperature (RT), followed by UV-irradiation for 1 hour at 365 nm. Samples were centrifuged at 10,000 rpm for 10 minutes at 4° C. to remove aggregates. Peptide exchange was determined by performing DSF analysis in triplicates (n=3), as previously described (44).

X-ray crystallography and structure determination. Purified HLA-A*11:01-A*02:01/HIV-1 RT and HLA-B*08:01-A*02:01/CMV complexes were concentrated to 12.5-15 mg/ml in SEC Buffer (150 mM NaCl, 25 mM Tris buffer, pH 8.0) and used for crystallization in 1:1 ratio of protein-crystallization buffer at 21° C. by sitting drops. Large plate crystals for HLA-A*11:01-A*02:01/HIV-1 RT were obtained in 0.02 M Sodium/Potassium phosphate, 0.1 M BIS-TRIS propane pH 8.5, 18-22% w/v PEG 3350 after 3 days. Small cubic crystals for HLA-B*08:01-A*02:01/CMV were obtained in 0.2 M Sodium fluoride, 0.1 M BIS-TRIS propane pH 8.5, 20-24% w/v PEG 3350 after 2 weeks. All crystals were harvested in crystallization buffer with 27% ethylene glycol using nylon cryo-loops (Hampton Research) and flash frozen in liquid nitrogen. Complete data collection was performed from single crystals under cryogenic conditions at Advanced Proton Source beamlines 19-ID-D and 24-ID-E for HLA-A*11:01-A*02:01/HIV-1 RT and B*08:01-A*02:01/CMV complexes, respectively. Diffraction images were indexed, integrated, and scaled using MOSFLM and HKL3000 in CCP4 Package. Structures were determined by molecular replacement method using Phaser and the previously published structure of HLA-A*02:01 (PDB ID: 5HHN) as a search model. Model building and refinement was performed using COOT and Phenix, respectively. Full data collection and refinement statistics are given in FIG. 21. Crystallographic figures were created using PyMOL.

Phylogenetic analysis. Multiple sequence alignments of the TCR-contact residues from approximately 10 most common allotypes from each subfamily HLA-A, -B, and -C, and of the α₁ and α₂ domains between the most similar wild-type alleles with the designed HLA-A*11:01-A*02:016M chimera were performed using ClustalOmega (46). Alignment files were further processed in ESPript (47). Phylogenetic trees were generated using best-fit models as calculated by MEGA7 (48) and processed in iTOL (49).

Biotinylation and tetramer formation. Biotinylation of the pMHC-I and soluble 10LH molecules was performed as previously described (50). In brief, BSP-tagged proteins were biotinylated using the BirA biotin-ligase bulk reaction kit (Avidity), according to the manufacturer's instructions. For the pMHC-I tetramer formation, Streptavidin-PE (Agilent Technologies, Inc.) at 4:1 monomer: streptavidin molar ratio was added to the biotinylated pMHC-I in the dark, every 10 min at room temperature over 10-time intervals.

Surface plasmon resonance. SPR experiments were conducted in duplicates or triplicates (n=2 or 3) using a BiaCore T200 instrument (Cytiva) in SPR buffer (50 mM NaCl, 20 mM sodium phosphate pH 7.2, 0.1% Tween-20). Approximately 650 resonance units (RU) of biotinylated-A*02:01/NY-ESO-1, A*02:01-B*08:01/NY-ESO-1, or the scFV 10LH were immobilized at 10 μL/min on a streptavidin-coated chip (GE Healthcare). TCR NYE-S1 or A*24:02/PHOX2B, and A*24:02-B*35:01/PHOX2B were captured on the coated surface followed by a wash-out step with buffer at desired concentrations. Samples were injected over the chip at 25° C. at a flow rate of 20 μL/min for 60 sec followed by a buffer wash with 180 sec dissociation time and equilibrium data were collected. The SPR sensorgrams, association/dissociation rate constants (kₐ, k_{d}) and equilibrium dissociation constant K_{D} values were analyzed in BiaCore T200 evaluation software (Cytiva) using kinetic analysis settings or fitted using one-site specific binding by GraphPad Prism v9. SPR sensorgrams and saturation curves were prepared in GraphPad Prism v9.

1G4 TOR lentivirus production. Lenti-X 293T cells (Takara) were cultured in DMEM (Gibco), 10% FBS (Gibco), and Glutamax (Gibco) and were plated one day before transfection. Cells were transfected at a confluency of 80-90% with TransIT-293 (Mirus) using pMD2.G (Addgene #12259, gift from Didier Trono), psPAX2 (Addgene #12260, gift from Didier Trono), and pSFFV-1G4. Virus-containing media was collected 24- and 48-hours post-transfection, clarified by centrifugation at 500 g for 10 min, and incubated with Lenti-X concentrator (Takara) for at least 24 hours. Virus was pooled and concentrated 50-100x, resuspended in PBS, aliquoted, and stored at −80° C. for subsequent T cell infections.

Primary human T cell tetramer staining. The studies involving human participants were reviewed and approved by the University of Pennsylvania review board. Written informed consent to participate in this study was provided by the participants. Healthy donor T cells were processed by the Human Immunology Core by magnetic separation of CD8+ T cells. Cells were cultured in Advanced RPMI (Gibco), 10% heat inactivated FBS (Gibco), Glutamax (Gibco), penicillin/streptomycin (Gibco), and 10 mM HEPES (Quality Biological), supplemented with 300 U/mL recombinant IL-2 (NCI Biological Resources Branch). T cells were maintained at ˜1 million cells/mL and were activated with a 1:1 ratio of Dynabeads Human T-Activator CD3/CD28 beads (Gibco) for 48 hours. 24 hours after initial activation, cells were either left untransduced or were transduced with lentivirus expressing the 1G4 TCR. Cells were debeaded by magnetic separation and expanded in the presence of IL-2. Transduction efficiency was determined by staining with an anti-VB13.1-APC antibody (Miltenyi Biotec.), typically greater than 50%. Cells were cryopreserved with CryoStor CS10 (StemCell Technologies). Thawed T cells were recovered and regrown in IL-2-containing complete medium for ˜3 days prior to staining. Cells were harvested and washed with PBS, 1% BSA, 2 mM EDTA with 5 g/mL PE-conjugated tetramers and incubated for 25 min at room temperature with mild agitation. After two washes with an RPMI-based buffer containing 1% FBS, cells were resuspended in 1:1000 Sytox Blue diluted in wash buffer to distinguish dead cells. Samples were processed on an LSR Fortessa (BD) and data analyzed by FlowJo v10.8.1.

Structural analysis reveals discrete HLA surfaces for peptide binding and TOR recognition. Applicants first sought to evaluate the degree of overlap between the residues which mediate interactions with the peptide and T cell receptor complementarity-determining regions, respectively. To do this, Applicants analyzed 384 pMHC-I structures from a curated, in-house database derived from the Protein Data Bank (HLA3DB; https://hla3db.research.chop.edu/) and 36 pMHC-TCR structures from the ATLAS database (51). For each pMHC-I structure, Applicants calculated a peptide-contact frequency as the percent of structures in which each position P of the first 180 amino acids comprising the peptide binding groove was within 4 Å from any peptide heavy atom (FIG. 22A). Likewise, Applicants calculated a TCR-contact frequency for each P using the available pMHC-TCR structures from the ATLAS database (FIG. 22B). Based on this analysis, Applicants classified MHC-I positions into three groups: i) peptide-only binding (PB) positions that primarily affect peptide binding with a non-zero peptide-contact frequency and a TCR-contact frequency less than 10%, ii) TCR-only binding (TB) positions which primarily affect TCR binding with a non-zero TCR-contact frequency and a peptide-contact frequency less than 10%, and iii) peptide-TCR binding (PTB) positions that affect both the peptide and TCR binding specificity with peptide- and TCR-contact frequencies greater than 10% (FIG. 22C and FIGS. 34A-34C). In cases where both frequencies were below 10%, Applicants selected the highest frequency to classify a given residue position as PB or TB. This analysis confirms that the HLA regions that mediate peptide binding show minimal overlap with TCR interaction surfaces.

Applicants next aimed to evaluate the degree of sequence variance among residues belonging to the three identified structural groups, towards understanding whether these positions could be modified to create synthetic molecules with specific binding properties. Therefore, Applicants aligned 2,896 sequences curated from the IMGT/HLA sequence database (53) using as reference the most common allotype HLA-A*02:01, and calculated a consensus score as the frequency of the most common amino acid at each position P. High consensus score implied highly conserved residues whereas low score suggested positions amendable to substitutions without compromising the stability of the pMHC-I complex (FIG. 22D). For instance, position 80 with a TCR-contact frequency of 5% and a peptide-contact frequency of 74% belongs in the PB category, whereas position 69 with frequencies of 89% and 13%, respectively, is implicated in the formation of more significant contacts with TCRs. Both positions are good targets for designing MHCs with novel peptide or TCR binding profiles, since they have low consensus scores (45% and 42%) and thus are highly polymorphic. On the other hand, nearly all the residues involved in the formation of hydrogen bond networks with the peptide main chain have a consensus score above 90%, implying strictly conserved interactions (52) (FIG. 22C and FIGS. 34A-34C). Notably, TB residues were overall more conserved, with the lowest consensus score at 67.3% (FIGS. 34A-34C), suggesting that the peptide- and TCR-contact residues followed distinct evolutionary paths to confer adaptability of interactions in the peptide binding groove. Taken together, Applicants demonstrate that results from both structural and sequence analysis can be used to define a set of MHC-I residues that could be altered to modify peptide binding while maintaining the MHC-TCR binding surface intact and vice versa.

Engineering chimeric MHC-I molecules using a structure-guided approach. Driven by the sequence and structural analysis, Applicants sought to explore the plasticity of existing HLA structures to accommodate novel peptides using a fixed-backbone design approach. Applicants developed a method called ‘CHaMeleon’, to generate synthetic molecules that combine the peptide binding specificity of one allele (template or groove allele) with the TCR binding surface of another (base allele). Applicants' approach takes as input an existing pHLA template structure and introduces a novel TCR binding surface in three steps: i) Generating a threaded model of a base allele sequence using a groove pHLA structural template, ii) Model optimization and binding energy analysis to identify the minimal set of mutations necessary to achieve an altered peptide binding specificity, and iii) experimental validation of the chimeric MHC-I refolded with the peptide that was observed in the original template structure of the groove allele (FIG. 23A).

First, Applicants used a 5 Å heavy atom distance threshold to define peptide contacting residues in the structure of a groove HLA with a known antigen, which would be used as a modeling template (FIG. 23B). Next, Applicants identified polymorphic residues which differ between the sequences of the groove and base alleles, and for all possible combinations of substitutions introduced on the base allele, Applicants threaded the corresponding protein sequences on the template structure. Applicants then performed energy optimization and assessed the stability of the resulting models by calculating the peptide: HLA interface energies using the Rosetta software (38) (FIGS. 39A-39F) and (FIGS. 23C-23D). This allowed Applicants to evaluate the effect of specific residues on the overall stability for each chimeric molecule and, subsequently, narrow down the selection of groove residues to a minimal set of substitutions that would confer binding to the provided peptide. As expected, for all cases the chimeric models were more stable than models of the threaded base sequence on the groove template, but less stable than the corresponding native groove structures (FIG. 35). For the top 2.5% structures with the lowest energies, Applicants calculated enrichment scores for each polymorphic position, which represent the fraction of top chimeric HLAs carrying a specific substitution for a groove allele residue. More specifically, positions with an enrichment score of 1.0 indicate substitutions that are present in all structures, whereas substitutions with very low or 0 enrichment scores most likely affect the overall stability of the pHLA complex and thus are not favorable (FIG. 23D). Additionally, mutations conferring different chemical properties at a certain position, such as a charged in the place of a neutral residue and vice versa, were always included in the minimal set whereas mutations replacing similar residues were excluded. To limit the number of substitutions impacting the TCR surface of the base allele, mutations in PTB positions were considered only if they contained a heavy atom within a more stringent threshold of 3.5 Å from the peptide. For the experimental validation of the designed chimeric HLAs, Applicants performed previously established protein refolding (54) using groove-specific peptides, stability measurements by differential scanning fluorimetry (DSF) analysis (55), and peptide binding assays in vitro (56) (FIG. 23E). Applicants' proposed rational approach for exploring combinations of groove specificities and TCR contact surfaces from naturally occurring MHC-I alleles provides the means to study the principles of pMHC-I/TCR recognition and assess TCR cross-reactivity, with important biomedical ramifications in the design of peptide-centric therapeutics.

Altering B- and F-Pocket specificities on HLA-A*02:01. Considering that the primary anchor positions for peptide binding onto MHC-I molecules are the P2 and P9 (20), Applicants employed the CHaMeleon approach to design synthetic pMHC-I molecules with altered peptide specificities by changing the B- and F-pockets of a base allele. For this purpose, Applicants used the common human HLA-A*02:01 allotype as base with a preference for hydrophobic residues at positions P2 and P9 (FIG. 24A and FIG. 27A). As structural templates, Applicants used the previously defined X-ray structures of HLA-A*11:01 (PDB ID: 1Q94) and C*07:02 (PDB ID: 5VGE) together with the high affinity, immunodominant peptide antigens HIV-1 RT (AIFQSSMTK; SEQ ID NO: 2) and RYR (RYRPGTVAL; SEQ ID NO: 3), respectively. These alleles show distinct peptide specificities with a preference for the charged Lys/Arg residues in the P9 anchor for HLA-A*11:01, and aromatic or charged residues in the P2 anchor for C*07:02 (FIG. 27B). Applicants identified and substituted 9 and 14 residues from HLA-A*11:01 and C*07:02 within the A*02:01 groove to generate the HLA-A*11:01-A*02:01 and C*07:02-A*02:01 chimeras, respectively (FIG. 20). Applicants next predicted the peptide specificities of the chimeric molecules (see Methods) and confirmed that the introduced amino acid substitutions resulted in altered peptide-binding specificities in positions P2 and P9, to resemble the sequence of the groove alleles (FIG. 24A). Comparison of the calculated energy values of the threaded structures showed that in both cases the chimeric molecules were more stable than the base but not the groove alleles (FIG. 35). Electrostatic surface potential analysis using the Rosetta models of each designed chimeric MHC-I, revealed altered surface charges of the HLA-A*02:01 groove, which are known to play a crucial role in selective peptide binding (35). As expected, the groove of HLA-A*11:01-A*02:01 was negatively charged, while HLA-C*07:02-A*02:01 changed to negatively charged A- and B-pockets but maintained a positively charged F-pocket (FIG. 24B).

To experimentally validate the designed chimeric HLAs, Applicants refolded HLA-A*11:01-A*02:01 and C*07:02-A02:01 with the HLA-A*11:01-specific HIV-1 RT and HLA-C*07:02-specific RYR peptides, respectively. In both cases Applicants were able to purify recombinant pMHC-I complexes by SEC (FIG. 27C) and further DSF analysis revealed melting temperatures characteristic of properly conformed peptide-bound molecules (Tₘ=51.8° C. for A*11:01-A*02:01/HIV-1 RT and 49.8° C. for C*07:02-A*02:01/RYR, FIG. 24C) (55). Taken together, Applicants' SEC and DSF results revealed that HLA groove-specific mutations can form properly folded and stable chimeric pMHC-I molecules after introducing target groove-specific peptides. Applicants then sought to determine whether these peptides adopted a similar conformation compared to their parental template HLA, considering that the conformation and mobility of the bound peptide could affect the affinity for TCR recognition (32, 57). While Applicants attempted to solve the crystal structures for both complexes, diffraction-quality crystals were obtained solely for the HLA-A*11:01-A*02:01/HIV-1 RT chimera. The best crystal diffracted to a 2.02 Å resolution and had clear electron density for the HIV-1 RT peptide, which Applicants modeled in the Fo-Fc electron density map (FIG. 21 and FIGS. 28A-28B). Overlay of the HIV-1 RT peptide from the wild-type HLA-A*11:01 versus the chimeric pMHC-I complex, revealed that both peptides adopted an identical backbone conformation with a deviation of 0.543 Å in RMSD values (FIG. 24D and FIG. 36). Additionally, Applicants observed that while the B-pocket was occupied by Ile2 which was principally stabilized through hydrogen bonds with the peptide main chain, the F-pocket was occupied by Lys9 projecting directly into the HLA groove (FIG. 24E). The observed accommodation of Lys9 into the F-pocket was the result of two salt bridge interactions between the Lys side chain and the introduced HLA-A*11:01 groove-specific residues Asp74 and Asp116 (FIG. 24E). These residues appeared to orient and stabilize the Lys9 side chain within the groove, while the main chain was further stabilized by hydrogen bonds with the HLA-A*11:01-specific Asp74 and A*02:01-specific Tyr84, Thr143, Lys146, and Trp147 (FIG. 24E). Interestingly, the introduced mutations Gln70 and Arg114 were responsible for forming multiple hydrogen bonds with Ser6 of the peptide within the C/D-pocket (FIG. 24E). While Applicants identified distinct HLA-A*11:01 groove-specific mutations crucial for peptide binding, several residues did not appear to be necessary for peptide association. Applicants, thus, hypothesized Applicants could optimize and refine the HLA-A*11:01-A*02:01 chimera, by re-engineering the HLA-A*02:01 base to introduce only six groove-specific mutations as opposed to the previous nine. This new six mutant HLA-A*11:01-A*02:01 (A*11:01-A*02:016M) chimera was not only capable of refolding with the HIV-1 RT peptide (FIG. 27C) but was also significantly more stable (Tₘ=59.8° C.) compared to the initial construct (Tₘ=51.8° C.) (FIG. 24C). Taken together, Applicants' HLA-A*11:01-A*02:01 structure revealed that the newly introduced peptide antigen adopted an identical conformation to that seen in the wild-type, parental HLA-A*11:01 structure (FIG. 36) (58), further validating Applicants' fixed-backbone design approach. Finally, based on the observed interactions with the peptide backbone, Applicants' design could be further optimized to improve pMHC-I complex stability.

Introducing a new P5 anchor within the C-Pocket of HLA-A*02:01. Naturally occurring HLA molecules can bind and display a wide distribution of peptide sequences (termed peptide repertoires), that consist of polar, hydrophobic, or charged amino acids at defined anchor positions. However, the peptide pools presented by known alleles do not cover the entire range of amino acid combinations on a peptide sequence, implying that the displayed repertoire at the population level contains blind spots of ‘forbidden’ peptides (22). Thus, Applicants explored further the applications of the CHaMeleon workflow to modify the set of binder peptides of an HLA molecule of interest, by introducing novel anchor positions within the HLA-A*02:01 groove. For this purpose, Applicants selected HLA-B*08:01 with a distinct preference for peptides with charged residues (Arg/Lys) at position P5 (FIG. 25A). To generate the HLA-B*08:01-A*02:01 chimera, a minimal set of 18 B*08:01-specific residues was identified and substituted within the A*02:01 groove based upon Rosetta threading and binding energy analysis, using the crystal structure of wild-type HLA-B*08:01 refolded with the CMV (ELNRKMIYM; SEQ ID NO: 1) peptide as a modeling template (PDB ID: 4QRT; FIG. 20). Applicants experimentally validated the ability of the designed chimeric HLA to form stable protein complexes with the desired CMV peptide, using in vitro refolding, purification and DSF analysis which revealed a Tₘ of 49.8° C. (FIG. 27D and FIG. 25B).

Applicants next examined whether the HLA-B*08:01-A*02:01 chimera could recapitulate the peptide-binding specificity of the groove allele Applicants used as a structural template, namely HLA-B*08:01. Applicants selected the HLA*B: 08:01 specific CMV and EBV (FLRGRAYGL; SEQ ID NO: 4), the A*02:01 specific TAX9 (LLFGYPVYV; SEQ ID NO: 25) and p90 (RLRGVYAAL; SEQ ID NO: 28), and the B*40:01 specific B40 (TEADVQQWL; SEQ ID NO: 32) peptides, as well as the H2-L^{d} specific p29 (YPNVNIHNF; SEQ ID NO: 31) epitope from the HIV gp120 protein, based on established epitopic sequences that were further validated by NetMHCPan4.0 predicted binding affinities (FIG. 37). Applicants then refolded the chimeric HLA with a B*08:01-specific photolabile peptide (EBV*=FLRGRAXGL (SEQ ID NO: 16), where X is the 3-amino-3-(2-nitrophenyl)-propionic acid) (43) with a Tₘ=48.2° C., to perform UV-mediated peptide exchange experiments (FIG. 27D and FIG. 25B) (44). Incubation with 10-fold molar excess of peptide followed by UV-irradiation led to an up-shift in the Tₘ peak for EBV (Tₘ=52.9° C.) (FIG. 25B), indicating the formation of stable pMHC-I molecules. Contrariwise, the p29 weak-binder peptide was unable to exchange (Tₘ=40.4° C.), demonstrating that the chimeric HLA groove is selective for HLA-B*08:01-specific peptides (FIG. 25B). Based on the sequence logo for HLA-B*08:01 peptide specificity profile (FIG. 25A), Applicants hypothesized that introduction of a charged residue in P5 of the weak-binder p29 peptide would enhance binding, and therefore designed the mutant peptide N5R p29 (p29^{N5R}, YPNVRIHNF; SEQ ID NO: 33). Notably, peptide exchange experiments with HLA-B*08:01-A*02:01/FLRGRAXGL (SEQ ID NO: 16) and excess of the mutant peptide resulted in a thermal shift of 23° C. compared to p29 (Tₘ=63.6° C. vs. 40.4° C., FIG. 25B), suggesting the formation of stable complexes. The p90 peptide showed very little exchange with a Tₘ of 37.9° C., while the A*02:01- and B*40:01-specific peptides TAX9 (SEQ ID NO: 25) and B40 were unable to exchange (FIG. 38). Altogether, Applicants' peptide exchange data further support that the HLA-B*08:01-A*02:01 chimera can preferably bind epitopes with high affinity for the binding groove of the template allele, namely B*08:01.

While Applicants were able to demonstrate that a synthetic MHC-I molecule with an additional P5 anchor could be designed and refolded, whether the B*08:01-specific peptide adopted an identical conformation compared to the wild-type template allele remained to be evaluated. Hence, Applicants attempted to solve the structure of HLA-B*08:01-A*02:01/CMV complex in an I23 space group and obtained crystals which diffracted to a 2.72 Å resolution (FIG. 21). As in the HLA-A*11:01-A*02:01 crystal structure, Applicants observed unambiguous electron densities for the CMV peptide that Applicants modeled within the Fo-Fc electron density map (FIGS. 29A-29B). Overlay of the CMV peptide bound to the wild-type HLA-B*08:01 and the B*08:01-A*02:01 chimera revealed an identical backbone conformation with a deviation of 0.495 Å in RMSD values between the two structures (FIG. 25C and FIG. 36), in agreement with Applicants' previous results for the HLA-A*11:01-A*02:01 chimera. While the F-pocket was occupied by Met9 and stabilized by hydrogen bonds along the main chain, the A-pocket was occupied by Glu1 which side chain interacted with the B*08:01-specific residues Arg62 and Asn63 (FIG. 25D). A strong electron density was observed for Lys5 within the C-pocket which formed three salt bridge interactions and one hydrogen bond with the B*08:01-specific residues Asp9, Asn70 and Asp74 (FIG. 25D), suggesting that these residues are crucial for stabilizing the peptide within the HLA groove. Altogether, these findings support the introduction of a novel P5 anchor within the HLA-A*02:01 groove to generate a chimeric molecule with a distinct peptide repertoire, without affecting the adopted conformation of the bound peptide.

Use of chimeric HLAs as molecular probes for identifying peptide-centric receptors. Applicants next sought to address whether Applicants can use chimeric HLAs to evaluate the extent to which interactions with specific TCRs or therapeutic antibodies are dependent upon interactions with HLA framework residues. Towards this goal, Applicants tested the wild-type TCRs 1G4 (31) and NYE-S1 (30) which recognize the tumor epitope NY-ESO-1 (SLLMWITQV; SEQ ID NO: 26) on HLA-A*02:01, as well as the peptide-centric engineered CAR 10LH that targets the neuroblastoma peptide PHOX2B (QYNPIRTTF; SEQ ID NO: 29) presented by A*24:02 (34). To design chimeric HLAs able to bind these epitopes on their non-physiological base Applicants, first, performed a phylogenetic analysis of the TCR contacting residues of selected HLA-A, -B, and -C allotypes to identify alleles with the most dissimilar TCR interacting surfaces compared to HLA-A*02:01 and A*24:02 (FIG. 26A). Based on the analysis, Applicants selected HLA-B*08:01 and B*35:01 to generate the HLA-A*02:01-B*08:01 and HLA-A*24:02-B*35:01 chimeras presenting the NY-ESO-1 and PHOX2B peptide antigens, respectively. Using the CHaMeleon approach, Applicants identified and introduced 11 HLA-A*02:01 and 16 A*24:02 residues in the peptide-binding grooves of B*08:01 and B*35:01, respectively (FIG. 20). Both chimeric molecules were successfully refolded with their respective target peptides (FIG. 26B) and, notably, the HLA-A*02:01-B*08:01 chimera was able to form a more stable complex with NY-ESO-1 compared to the wild-type A*02:01 (Tₘ=65.2° C. vs. Tₘ=62.0° C.), as revealed by DSF experiments (FIG. 26C). Contrariwise, the HLA-A*24:02-B*35:01 chimera was destabilized by almost 15° C. compared to the wild-type A*24:02 (Tₘ=48.3° C. vs. Tₘ=65.9° C.), although was still able to form loaded pMHC-I complexes (FIG. 26C).

To test the hypothesis, Applicants stained primary CD8+ T cells transduced with the wild-type TCR 1G4 that recognizes the NY-ESO-1 peptide presented by A*02:01 (31) (FIG. 30), and generated phycoerythrin (PE) tetramers of HLA-A*02:01/NY-ESO-1 and A*02:01-B*08:01/NY-ESO-1, as previously described (59). As a negative control, Applicants used HLA-A*02:01 refolded with the NY-ESO-1 peptide carrying an Ala substitution in position 5, namely NY-ESO-1^{W5A} (SLLMAITQV; SEQ ID NO: 27), which has been shown to be essential for TCR recognition (60). Analysis by flow cytometry revealed lack of staining with HLA-A*02:01/NY-ESO-1^{W5A} and A*02:01-B*08:01/NY-ESO-1 compared to the wild-type A*02:01/NY-ESO-1 tetramers (FIG. 26D). These results confirm that TCR 1G4 recognizes specific peptide: HLA antigens in a highly restricted manner (61), as interactions were disrupted both in the case of the wild-type MHC-I presenting a peptide with a single amino acid substitution and the chimeric pMHC-I presenting the target peptide. Applicants, next, used the newly characterized NYE-S1 TCR selective for HLA-A*02:01/NY-ESO-1 (30) to quantitively assess pMHC-I/TCR interactions using surface plasmon resonance (SPR) experiments. Soluble NYE-S1 bound weakly to immobilized HLA-A*02:01/NY-ESO-1 with a dissociation equilibrium constant K_{D}=4.9 μM, in agreement with previous studies (30), but was unable to interact with both HLA-A*02:01/NY-ESO-1^{W5A} and A*02:01-B*08:01/NY-ESO-1 chimeric molecules (FIG. 26E and FIGS. 31A-31B). Additionally, Applicants tested the scFv-based CAR 10LH, which is selective for A*24:02/PHOX2B and has been shown to interact with this specific epitope even when presented by different HLAs, i.e. HLA-A*23:01 and B*14:02 (34). As a negative control, Applicants used HLA-A*24:02 refolded with PHOX2B peptide carrying an Ala substitution in P6, namely PHOX2BR6A, which completely disrupts interactions with 10LH (34). As expected, 10LH bound to HLA-A*24:02 presenting the wild-type PHOX2B peptide with a K_{D} of 11.1 nM but not the mutated PHOX2BR6A (FIG. 26E and FIGS. 31C-31D). Notably, the chimeric HLA-A*24:02-B*35:01/PHOX2B and 10LH interactions were 20-fold weaker with an estimated nanomolar range K_{D} compared to the wild-type (FIG. 26E and FIGS. 31E-31F). However, the observed 200 nanomolar binding still falls within the affinity range (up to micromolar) for TCRs/CARs and their pHLA targets which has been demonstrated to sufficiently trigger T cell killing (62, 63).

To explore the structural basis of the loss of TCR recognition for the chimeric pMHC-I molecules, Applicants compared the TCR-interacting surfaces of the generated chimeric models. Applicants observed that 6 out of 8 polymorphic TCR residues for HLA-A*02:01-B*08:01 and 7 out of 10 for A*24:02-B*35:01 chimeras were residues of the base allele and could, thus, affect TCR/CAR recognition (FIG. 26F). To further determine which HLA-B*08:01 base residues were responsible for the loss of NYE-S1 recognition, Applicants compared them to the A*02:01 residues responsible for TCR binding based on the solved crystal structures of HLA-A*02:01/NY-ESO-1 with the TCRs 1G4 and NYE-S1 (30, 31). Applicants identified the HLA-A*02:01 residue Arg65 to be important for 1G4 and NYE-S1 binding along the α₁ helix, forming interactions with Asp55 and Asp67 of the CDR2β loops, respectively (FIG. 26G). In HLA-A*02:01-B*08:01 chimera, this residue was replaced by Gln65 of the wild-type B*08:01, suggesting that disruption of these interactions is crucial for TCR binding. Interestingly, the same position differs between HLA-A*24:02 and B*35:01 (FIG. 26F), however had no effect on 10LH recognition, as expected for the peptide-centric CARs which are not constrained by the germline-encoded CDR1-2/MHC interactions. Taken together, Applicants' cell-based and biophysical data confirm that the peptide antigen alone is not sufficient to maintain known pMHC-I/TCR interactions when presented in the context of a divergent HLA framework surface and suggest that loss of binding can occur even with a single amino acid substitution on the MHC-I/TCR interacting surface. In contrast, recognition by the peptide-centric CAR 10LH was not disrupted, highlighting the potential of scFV-based immunotherapies to target a broad range of allotypes.

The highly polymorphic nature of the MHC-I peptide binding groove highlights a stable structural scaffold which can be adapted to accommodate a diverse panel of ligands (6). While human MHC-I allotypes encompass a plethora of peptide binding specificities, there remain gaps in the repertoire of antigens which can be recognized and displayed by the existing HLA proteins (20, 22). On the other hand, TCRs can recognize different peptide: MHC-I complexes through a combination of peptide-centric and germline contacts with MHC-I framework residues and are limited to a restricted range of interactions with HLAs. Here, Applicants outline a systematic approach to generate synthetic MHC-I molecules blending desired peptide and TCR interaction properties. The analysis shows that Applicants can use existing structural information to discern MHC-I residues responsible for peptide binding and TCR recognition, enabling the design of chimeric molecules according to a fixed-backbone protocol that is guided by a structural template. Applicants provide biochemical evidence that the HLA pockets within the groove can be altered to accommodate new peptides while maintaining the TCR surface features of a specific HLA allotype. Applicants' approach is further validated by the solved crystal structures for two chimeric MHC-I molecules, which reveal that the peptide is presented in the specified conformation. Notably, all-atom RMSD values between the crystal structure and the Rosetta model were below 2 Å both for the peptide and MHC-I α₁/α₂ domains (FIG. 36). Finally, functional characterization using in vitro tetramer staining and biophysical binding experiments demonstrates the practical utility of Applicants' chimeric molecules as screening tools to evaluate peptide-centric interactions with T cell receptors and therapeutic antibodies, respectively.

Applicants' work offers insights into principles underpinning the molecular evolution of MHC-I allotypes, and the emergence of distinct supertypes (7). Owning to the stability and malleability of the MHC-I scaffold, a minimal set of amino acid substitutions can lead to drastic changes in peptide binding preference, and thereby supertype divergence (64). It is worth noting that for some of the chimeric molecules designed in this Example, Applicants can identify known HLA allotypes with similar peptide-binding groove sequences and assumed peptide binding preferences. In particular, the HLA-A*11:01-A*02:01 chimera, designed to accommodate peptides with positively charged P9 residues, is similar in sequence (4 amino acid differences among peptide-binding residues) to the known allotypes HLA-A*03:05 and A*03:17 (A03 supertype) (64) that have acidic F-pockets, and therefore are predicted to bind positively charged peptides (FIGS. 32A-32C). Likewise, the designed HLA-A*11:01-A*02:01^{6M} chimera possessing the groove of A*11:01 (A03 supertype), differs in 4 peptide-binding residues with each of the HLA-A*02:35 and A*02:246 allotypes (A02 supertype) (64) (FIGS. 32A-32C). Interestingly, a combination of all substitutions from the wild-type alleles, where two of them are shared, results in Applicants' computationally designed chimeric sequence (FIGS. 32A-32C). This in turn suggests that Applicants' synthetic molecules incorporate features from distinct supertypes that could naturally occur over time and represents an example of convergent evolution between A03 and A02 supertypes. However, there is no structural evidence that these allotypes bind the peptides in a similar backbone conformation compared to the wild-type template allele. Applicants' study also describes a chimeric HLA, namely HLA-B*08:01-A*02:01, with no direct equivalent amongst naturally occurring HLAs (15 amino acid differences with the closest allotype). This could be either due to lack of sequence data on already existing allotypes in the population, or because this specific peptide binding motif has not yet been sampled by the ongoing evolutionary process for A02 alleles. In summary Applicants' designed molecules provide evidence that barriers between different supertypes are low and provide an avenue for creating novel allotypes which are not represented in the existing HLA repertoires.

Chimeric MHC molecules designed with desired peptide-binding grooves and TCR-interacting surfaces have potential immune system engineering applications towards the development of targeted therapies for breaking tolerance for weak disease- or cancer-associated antigens. Current approaches to break self-tolerance include the use of altered peptide ligands for personalized cancer vaccines (65, 66), and the introduction of checkpoint inhibitors to overcome peripheral tolerance (67). A recent study has shown that introduction of point mutations at the TCR binding interface of native MHCs presenting tumor-associated antigens can be used to activate T cells through allorecognition (33). Using the CHaMeleon approach outlined in this work, Applicants can introduce novel anchor positions to the peptide-binding groove of selected MHCs and generate chimeric molecules presenting established tumor-associated antigens with modified TCR interaction surfaces, relative to a specific HLA allotype. These chimeric HLAs can be then used as immunogens, to elicit alloreactive T cell responses for self-antigens that are upregulated in cancer (68). In a similar manner, epitope-focused vaccination strategies are based on eliciting antibodies towards non-immunogenic antigens with multiple applications against diseases and cancer therapy (69, 70). More importantly, with the advent of CAR-T cell therapies (71), there has been an increasing interest in designing peptide-centric receptors that are highly specific for a certain peptide sequence and are relatively tolerant to amino acid substitutions of HLA framework residues within the peptide: MHC complex (34). As implied by Applicants' proof of concept in vitro binding studies, chimeric MHC-I molecules can serve as screening tools to identify peptide-centric CARs for specific antigens. When prepared in tetramerized form and used as selection markers in existing directed evolution and antibody panning approaches (72), chimeric peptide: MHC complexes can enable the development of therapies which can cover larger cohorts of patients.

Collectively, the results suggest that Applicants are capable of re-capitulating and potentially expanding the antigen presentation profile of target alleles through a structure-guided, systematic redesign of the MHC-I peptide binding groove. Applicants' approach serves as a toehold for understanding the molecular evolution and functional divergence of HLA allotypes, while also providing useful screening tools to facilitate the development of tolerance-breaking vaccines and targeted CAR-T therapies.

The datasets presented in this Example can be found in online repositories. The names of the repository/repositories and accession number(s) can be found below: http://www.wwpdb.org/, 8ERX; http://www.wwpdb.org/, 8ESH.

### REFERENCES

1. Zimmer J, Andres E, Donato L, Hanau D, Hentges F, de la Salle H. Clinical and immunological aspects of HLA class I deficiency. QJM: Int J Med (2005) 98:719-27. doi: 10.1093/qjmed/hci112
2. Blum J S, Wearsch P A, Cresswell P. Pathways of antigen processing. Annu Rev Immunol (2013) 31:443-73. doi: 10.1146/annurev-immunol-032712-095910
3. Riedhammer C, Weissert R. Antigen presentation, autoantigens, and immune regulation in multiple sclerosis and other autoimmune diseases. Front Immunol (2015) 6:322. doi: 10.3389/fimmu.2015.00322
4. Koutsakos M, Illing P T, Nguyen T H O, Mifsud N A, Crawford J C, Rizzetto S, et al. Human CD8+ T cell cross-reactivity across influenza a, b and c viruses. Nat Immunol (2019) 20:613-25. doi: 10.1038/s41590-019-0320-6
5. Dhatchinamoorthy K, Colbert J D, Rock K L. Cancer immune evasion through loss of MHC class I antigen presentation. Front Immunol (2021) 12:636568.

doi: 10.3389/fimmu.2021.636568
6. Robinson J, Guethlein L A, Cereb N, Yang S Y, Norman P J, Marsh S G E, et al. Distinguishing functional polymorphism from random variation in the sequences of >10,000 HLA-a, -b and -c alleles. PloS Genet (2017) 13: e1006862. doi: 10.1371/journal.pgen.1006862
7. Vita R, Mahajan S, Overton J A, Dhanda S K, Martini S, Cantrell J R, et al. The immune epitope database (IEDB): 2018 update. Nucleic Acids Res (2019) 47: D339-43. doi: 10.1093/nar/gky1006
8. Norman P J, Norberg S J, Guethlein L A, Nemat-Gorgani N, Royce T, Wroblewski E E, et al. Sequences of 95 human MHC haplotypes reveal extreme coding variation in genes other than highly polymorphic HLA class i and II. Genome Res (2017) 27:813-23. doi: 10.1101/gr.213538.116
9. Serçinoğlu O, Ozbek P. Sequence-structure-function relationships in class I MHC: A local frustration perspective. PloS One (2020) 15: e0232849. doi: 10.1371/journal.pone.0232849
10. Barker D J, Maccari G, Georgiou X, Cooper M A, Flicek P, Robinson J, et al. The IPD-IMGT/HLA database. Nucleic Acids Res (2022), 51: D1053-D1060. doi: 10.1093/nar/gkac1011
11. Penn D J, Damjanovich K, Potts WK. MHC heterozygosity confers a selective advantage against multiple-strain infections. Proc Natl Acad Sci USA (2002) 99:11260-4. doi: 10.1073/pnas. 162006499
12. Zernich D, Purcell A W, Macdonald W A, Kjer-Nielsen L, Ely L K, Laham N, et al. Natural HLA class I polymorphism controls the pathway of antigen presentation and susceptibility to viral evasion. J Exp Med (2004) 200:13-24. doi: 10.1084/jem.20031680
13. Prugnolle F, Manica A, Charpentier M, Guégan J F, Guernier V, Balloux F. Pathogen-driven selection and worldwide HLA class I diversity. Curr Biol (2005) 15:1022-7. doi: 10.1016/j.cub.2005.04.050
14. Cagliani R, Sironi M. Pathogen-driven selection in the human genome. Int J Evol. Biol (2013) 2013:1-6. doi: 10.1155/2013/204240
15. Falk K, Rotzschke O, Stevanovie S, Jung G, Rammensee H-G. Allele-specific motifs revealed by sequencing of self-peptides eluted from MHC molecules. Nature (1991) 351:290-6. doi: 10.1038/351290a0
16. Yewdell J W, Bennink J R. Immunodominance in major histocompatibility complex class I-restricted T lymphocyte responses. Annu Rev Immunol (1999) 17:51-88. doi: 10.1146/annurev.immunol.17.1.51
17. Adams E J, Luoma A M. The adaptable major histocompatibility complex (MHC) fold: Structure and function of nonclassical and MHC class I-like molecules. Annu Rev Immunol (2013) 31:529-61. doi: 10.1146/annurev-immunol-032712-095912
18. Bjorkman P J, Saper M A, Samraoui B, Bennett W S, Strominger J L, Wiley D C. Structure of the human class I histocompatibility antigen, HLA-A2. Nature (1987) 329:506-12. doi: 10.1038/329506a0
19. van Deutekom H W M, Keşmir C. Zooming into the binding groove of HLA molecules: which positions and which substitutions change peptide binding most? Immunogenetics (2015) 67:425-36. doi: 10.1007/s00251-015-0849-y
20. Rasmussen M, Harndahl M, Stryhn A, Boucherma R, Nielsen L L, Lemonnier F A, et al. Uncovering the peptide-binding specificities of HLA-c: A general strategy to determine the specificity of any MHC class I molecule. JI (2014) 193:4790-802. doi: 10.4049/jimmunol. 1401689
21. Smith A R, Alonso J A, Ayres C M, Singh N K, Hellman L M, Baker B M. Structurally silent peptide anchor modifications allosterically modulate T cell recognition in a receptor-dependent manner. Proc Natl Acad Sci USA (2021) 118: e2018125118. doi: 10.1073/pnas.2018125118
22. Lee K-H, Chang Y-C, Chen T-F, Juan H-F, Tsai H-K, Chen C-Y. Connecting MHC-i-binding motifs with HLA alleles via deep learning. Commun Biol (2021) 4:1194. doi: 10.1038/s42003-021-02716-8
23. Van Laethem F, Tikhonova A N, Singer A. MHC restriction is imposed on a diverse T cell receptor repertoire by CD4 and CD8 co-receptors during thymic selection. Trends Immunol (2012) 33:437-41. doi: 10.1016/j.it.2012.05.006
24. Marrack P, Scott-Browne J P, Dai S, Gapin L, Kappler J W. Evolutionarily conserved amino acids that control TCR-MHC interaction. Annu Rev Immunol (2008) 26:171-203. doi: 10.1146/annurev.immunol.26.021607.090421
25. Christopher Garcia K, Adams J J, Feng D, Ely L K. The molecular basis of TCR germline bias for MHC is surprisingly simple. Nat Immunol (2009) 10:143-7. doi: 10.1038/ni.f.219
26. Arstila T P, Casrouge A, Baron V, Even J, Kanellopoulos J, Kourilsky P. A direct estimate of the human alphabeta T cell receptor diversity. Science (1999) 286:958-61. doi: 10.1126/science.286.5441.958
27. Wooldridge L, Ekeruche-Makinde J, van den Berg H A, Skowera A, Miles J J, Tan M P, et al. A single autoimmune T cell receptor recognizes more than a million different peptides. J Biol Chem (2012) 287:1168-77. doi: 10.1074/jbc.M111.289488
28. Blevins S J, Pierce B G, Singh N K, Riley T P, Wang Y, Spear T T, et al. How structural adaptability exists alongside HLA-A2 bias in the human αβ TCR repertoire. Proc Natl Acad Sci USA (2016) 113 (9): E1276-85. doi: 10.1073/pnas. 1522069113
29. Rudolph M G, Stanfield R L, Wilson I A. HOW TCRS BIND MHCS, PEPTIDES, AND CORECEPTORS. Annu Rev Immunol (2006) 24:419-66. doi: 10.1146/annurev.immunol.23.021704.115658
30. Coles C H, Mulvaney R M, Malla S, Walker A, Smith K J, Lloyd A, et al. TCRs with distinct specificity profiles use different binding modes to engage an identical peptide-HLA complex. JI (2020) 204:1943-53. doi: 10.4049/jimmunol. 1900915
31. Chen J-L, Stewart-Jones G, Bossi G, Lissin N M, Wooldridge L, Choi E M L, et al. Structural and kinetic basis for heightened immunogenicity of T cell vaccines. J Exp Med (2005) 201:1243-55. doi: 10.1084/jem.20042323
32. Saito N G, Chang H C, Paterson Y. Recognition of an MHC class I-restricted antigenic peptide can be modulated by para-substitution of its buried tyrosine residues in a TCR-specific manner. J Immunol (1999) 162:5998-6008. doi: 10.4049/jimmunol. 162.10.5998
33. Parks C A, Henning K R, Pavelko K D, Hansen M J, Van Keulen V P, Reed B K, et al. Breaking tolerance with engineered class I antigen-presenting molecules. Proc Natl Acad Sci USA (2019) 116:3136-45. doi: 10.1073/pnas. 1807465116
34. Yarmarkovich M, Marshall Q F, Warrington J M, Premaratne R, Farrel A, Groff D, et al. Cross-HLA targeting of intracellular oncoproteins with peptide-centric CARs. Nature (2021) 599:477-84. doi: 10.1038/s41586-021-04061-6
35. Nguyen A T, Szeto C, Gras S. The pockets guide to HLA class I molecules. Biochem Soc Trans (2021) 49:2319-31. doi: 10.1042/BST20210410
36. Candia M, Kratzer B, Pickl W F. On peptides and altered peptide ligands: From origin, mode of action and design to clinical application (Immunotherapy). Int Arch Allergy Immunol (2016) 170:211-33. doi: 10.1159/000448756
37. Hoppes R, Oostvogels R, Luimstra J J, Wals K, Toebes M, Bies L, et al. Altered peptide ligands revisited: Vaccine design through chemically modified HLA-A2-restricted T cell epitopes. JI (2014) 193:4803-13. doi: 10.4049/jimmunol.1400800
38. Leman J K, Weitzner B D, Lewis S M, Adolf-Bryfogle J, Alam N, Alford R F, et al. Macromolecular modeling and design in Rosetta: recent methods and frameworks. Nat Methods (2020) 17:665-80. doi: 10.1038/s41592-020-0848-2
39. Reynisson B, Alvarez B, Paul S, Peters B, Nielsen M. NetMHCpan-4.1 and NetMHCIIpan-4.0: improved predictions of MHC antigen presentation by concurrent motif deconvolution and integration of MS MHC eluted ligand data. Nucleic Acids Res (2020) 48: W449-54. doi: 10.1093/nar/gkaa379
40. Thomsen MCF, Nielsen M. Seq2Logo: a method for construction and visualization of amino acid binding motifs and sequence profiles including sequence weighting, pseudo counts and two-sided representation of amino acid enrichment and depletion. Nucleic Acids Res (2012) 40: W281-287. doi: 10.1093/nar/gks469
41. Li H, Natarajan K, Malchiodi E L, Margulies D H, Mariuzza R A. Three-dimensional structure of h-2Dd complexed with an immunodominant peptide from human immunodeficiency virus envelope glycoprotein 120. J Mol Biol (1998) 283:179-91. doi: 10.1006/jmbi. 1998.2091
42. Gasteiger E, Gattiker A, Hoogland C, Ivanyi I, Appel R D, Bairoch A. ExPASy: The proteomics server for in-depth protein knowledge and analysis. Nucleic Acids Res (2003) 31:3784-8. doi: 10.1093/nar/gkg563
43. Toebes M, Coccoris M, Bins A, Rodenko B, Gomez R, Nieuwkoop N J, et al. Design and use of conditional MHC class I ligands. Nat Med (2006) 12:246-51. doi: 10.1038/nm1360
44. Rodenko B, Toebes M, Hadrup S R, van Esch W J E, Molenaar A M, Schumacher T N M, et al. Generation of peptide-MHC class I complexes through UV-mediated ligand exchange. Nat Protoc (2006) 1:1120-32. doi: 10.1038/nprot.2006.121
45. Chen V B, Arendall W B, Headd J J, Keedy D A, Immormino R M, Kapral G J, et al. MolProbity: All-atom structure validation for macromolecular crystallography. Acta Crystallogr D Biol Crystallogr (2010) 66:12-21. doi: 10.1107/S0907444909042073
46. Sievers F, Wilm A, Dineen D, Gibson T J, Karplus K, Li W, et al. Fast, scalable generation of high-quality protein multiple sequence alignments using clustal omega. Mol Syst Biol (2011) 7:539. doi: 10.1038/msb.2011.75
47. Robert X, Gouet P. Deciphering key features in protein structures with the new ENDscript server. Nucleic Acids Res (2014) 42: W320-324. doi: 10.1093/nar/gku316
48. Kumar S, Stecher G, Tamura K. MEGA7: Molecular evolutionary genetics analysis version 7.0 for bigger datasets. Mol Biol Evol (2016) 33:1870-4. doi: 10.1093/molbev/msw054
49. Letunic I, Bork P. Interactive tree of life (iTOL) v5: An online tool for phylogenetic tree display and annotation. Nucleic Acids Res (2021) 49: W293-6. doi: 10.1093/nar/gkab301
50. Overall S A, Toor J S, Hao S, Yarmarkovich M, Sara M, O'Rourke, et al. High throughput pMHC-I tetramer library production using chaperone-mediated peptide exchange. Nat Commun (2020) 11:1909. doi: 10.1038/s41467-020-15710-1
51. Borrman T, Cimons J, Cosiano M, Purcaro M, Pierce B G, Baker B M, et al. ATLAS: A database linking binding affinities with structures for wild-type and mutant TCR-pMHC complexes: Linking TCR-pMHC affinities with structure. Proteins (2017) 85:908-16. doi: 10.1002/prot.25260
52. Wu Y, Zhang N, Wei X, Lu S, Li S, Hashimoto K, et al. The structure of a peptide-loaded shark MHC class I molecule reveals features of the binding between β 2-microglobulin and h chain conserved in evolution. JI (2021) 207:308-21. doi: 10.4049/jimmunol.2001165
53. Robinson J. IMGT/HLA and IMGT/MHC: Sequence databases for the study of the major histocompatibility complex. Nucleic Acids Res (2003) 31:311-4. doi: 10.1093/nar/gkg070
54. Garboczi D N, Hung D T, Wiley D C. HLA-A2-peptide complexes: Refolding and crystallization of molecules expressed in Escherichia coli and complexed with single antigenic peptides. Proc Natl Acad Sci USA (1992) 89 (8): 3429-33. doi: 10.1073/pnas.89.8.3429
55. Hellman L M, Yin L, Wang Y, Blevins S J, Riley T P, Belden O S, et al. Differential scanning fluorimetry based assessments of the thermal and kinetic stability of peptide-MHC complexes. J Immunol Methods (2016) 432:95-101. doi: 10.1016/j.jim.2016.02.016
56. Bakker A H, Hoppes R, Linnemann C, Toebes M, Rodenko B, Berkers C R, et al. Conditional MHC class I ligands and peptide exchange technology for the human MHC gene products HLA-A1, -A3, -A11, and -B7. Proc Natl Acad Sci (2008) 105:3825-30. doi: 10.1073/pnas.0709717105
57. Rossjohn J, Gras S, Miles J J, Turner S J, Godfrey D I, Mccluskey J. T Cell antigen receptor recognition of antigen-presenting molecules. Annu Rev Immunol (2015) 33:169-200. doi: 10.1146/annurev-immunol-032414-112334
58. Li L, Bouvier M. Structures of HLA-A*1101 complexed with immunodominant nonamer and decamer HIV-1 epitopes clearly reveal the presence of a middle, secondary anchor residue. J Immunol (2004) 172:6175-84. doi: 10.4049/jimmunol. 172.10.6175
59. Altman J D, Moss P A H, Goulder P J R, Barouch D H, McHeyzer-Williams M G, Bell J I, et al. Phenotypic analysis of antigen-specific T lymphocytes. Science (1996) 274:94-6. doi: 10.1126/science.274.5284.94
60. Ishihara M, Kitano S, Kageyama S, Miyahara Y, Yamamoto N, Kato H, et al. NY-ESO-1-specific redirected T cells with endogenous TCR knockdown mediate tumor response and cytokine release syndrome. J Immunother Cancer (2022) 10: e003811. doi: 10.1136/jitc-2021-003811
61. Archbold J K, Ely L K, Kjer-Nielsen L, Burrows S R, Rossjohn J, Mccluskey J, et al. T Cell allorecognition and MHC restriction-a case of Jekyll and Hyde? Mol Immunol (2008) 45:583-98. doi: 10.1016/j.molimm.2006.05.018
62. Natarajan K, McShan A C, Jiang J, Kumirov V K, Wang R, Zhao H, et al. An allosteric site in the T-cell receptor cβ domain plays a critical signalling role. Nat Commun (2017) 8:15260. doi: 10.1038/ncomms15260
63. Laugel B, van den Berg H A, Gostick E, Cole D K, Wooldridge L, Boulter J, et al. Different T cell receptor affinity thresholds and CD8 coreceptor dependence govern cytotoxic T lymphocyte activation and tetramer binding properties. J Biol Chem (2007) 282:23799-810. doi: 10.1074/jbc.M700976200
64. Sidney J, Peters B, Frahm N, Brander C, Sette A. HLA class I supertypes: a revised and updated classification. BMC Immunol (2008) 9:1. doi: 10.1186/1471-2172-9-1
65. Ott P A, Hu Z, Keskin D B, Shukla S A, Sun J, Bozym D J, et al. An immunogenic personal neoantigen vaccine for patients with melanoma. Nature (2017) 547:217-21. doi: 10.1038/nature22991
66. Sahin U, Derhovanessian E, Miller M, Kloke B-P, Simon P, Lower M, et al. Personalized RNA mutanome vaccines mobilize poly-specific therapeutic immunity against cancer. Nature (2017) 547:222-6. doi: 10.1038/nature23003
67. Ribas A, Wolchok JD. Cancer immunotherapy using checkpoint blockade. Science (2018) 359:1350-5. doi: 10.1126/science.aar4060
68. Bright R K, Bright J D, Byrne J A. Overexpressed oncogenic tumor-self antigens. Hum Vaccin Immunother (2014) 10:3297-305. doi: 10.4161/hv.29475
69. Oscherwitz J. The promise and challenge of epitope-focused vaccines. Hum Vaccines Immunother. (2016) 12:2113-6. doi: 10.1080/21645515.2016.1160977
70. Correia B E, Bates J T, Loomis R J, Baneyx G, Carrico C, Jardine J G, et al. Proof of principle for epitope-focused vaccine design. Nature (2014) 507:201-6. doi: 10.1038/nature12966
71. June C H, Riddell S R, Schumacher T N. Adoptive cellular therapy: a race to the finish line. Sci Transl Med (2015) 7: 280ps7. doi: 10.1126/scitranslmed.aaa3643
72. Beasley M D, Niven K P, Winnall W R, Kiefel B R. Bacterial cytoplasmic display platform retained display (ReD) identifies stable human germline antibody frameworks. Biotechnol J (2015) 10:783-9. doi: 10.1002/biot.201400560

### Abbreviation Definition

ATLAS databse Accounting Transaction Ledger Archival System database
β2m β2-microglobulin
BSP BirA Substrate Peptide
CARs Chimeric Antigen Receptors
CD8+ T cells Cytotoxic T cell
CDRs Complementarity-Determining Regions
CMV Cytomegalovirus
DSF Differential Scanning Fluorimetry
EBV Eppstein-Barr virus
ER Endoplasmic Reticulum
HIV Human Immunodeficiency virus
HLAs Human Leucocyte Antigens
IMGT/HLA database ImMunoGeneTics project/HLA database
MHC-I Major Histocompatibility Complex class I
PB positions Peptide-only binding positions
PDB Protein Data Bank
PDB ID Protein Data Bank Identification number
pHLA peptide-HLA complex
pMHC-I peptide-MHC-I complex
PTB positions peptide-TCR-binding positions
RMSD Root Mean Square Deviation
scFv Single-Chain Fragment Variable
SEC Size-Exclusion Chromatography
SPR Surface Plasmon Resonance
TB positions TCR-only binding positions
TCRs T cell receptors
UV Ultraviolet radiations

### ADDITIONAL REFERENCES

Androlewicz, M. J. (1999). The role of tapasin in MHC class I antigen assembly. Immunol Res 20, 79-88.
Berman, H. M., Westbrook, J., Feng, Z., Gilliland, G., Bhat, T. N., Weissig, H., Shindyalov, I. N., and Bourne, P. E. (2000). The Protein Data Bank. Nucleic Acids Res 28, 235-242.
Blum, J. S., Wearsch, P. A., and Cresswell, P. (2013). Pathways of antigen processing. Annu Rev Immunol 31, 443-473.
contributors, W. Antigen presentation (Wikipedia, The Free Encyclopedia).
Dhatchinamoorthy, K., Colbert, J. D., and Rock, K. L. (2021). Cancer Immune Evasion Through Loss of MHC Class I Antigen Presentation. Front Immunol 12, 636568.
Falk, K., Rotzschke, O., Deres, K., Metzger, J., Jung, G., and Rammensee, H. G. (1991). Identification of naturally processed viral nonapeptides allows their quantification in infected cells and suggests an allele-specific T cell epitope forecast. J Exp Med 174, 425-434.
Garboczi, D. N., Hung, D. T., and Wiley, D. C. (1992). HLA-A2-peptide complexes: refolding and crystallization of molecules expressed in Escherichia coli and complexed with single antigenic peptides. Proc Natl Acad Sci USA 89, 3429-3433.
Hammer, G. E., Gonzalez, F., Champsaur, M., Cado, D., and Shastri, N. (2006). The aminopeptidase ERAAP shapes the peptide repertoire displayed by major histocompatibility complex class I molecules. Nat Immunol 7, 103-112.
Hansen, T. H., Connolly, J. M., Gould, K. G., and Fremont, D. H. (2010). Basic and translational applications of engineered MHC class I proteins. Trends Immunol 31, 363-369.
Hellman, L. M., Yin, L., Wang, Y., Blevins, S. J., Riley, T. P., Belden, O. S., Spear, T. T., Nishimura, M. I., Stern, L. J., and Baker, B. M. (2016). Differential scanning fluorimetry based assessments of the thermal and kinetic stability of peptide-MHC complexes. J Immunol Methods 432, 95-101. Jones, L. L., Brophy, S. E., Bankovich, A. J., Colf, L. A., Hanick, N. A., Garcia, K. C.,
and Kranz, D. M. (2006). Engineering and characterization of a stabilized alpha1/alpha2 module of the class I major histocompatibility complex product Ld. J Biol Chem 281, 25734-25744.
Koutsakos, M., McWilliam, H. E. G., Aktepe, T. E., Fritzlar, S., Illing, P. T., Mifsud, N. A., Purcell, A. W., Rockman, S., Reading, P. C., Vivian, J. P., et al. (2019). Downregulation of MHC Class I Expression by Influenza A and B Viruses. Front Immunol 10, 1158.
Lee, K. H., Chang, Y. C., Chen, T. F., Juan, H. F., Tsai, H. K., and Chen, C. Y. (2021). Connecting MHC-I-binding motifs with HLA alleles via deep learning. Commun Biol 4, 1194.
Li, H., Natarajan, K., Malchiodi, E. L., Margulies, D. H., and Mariuzza, R. A. (1998). Three-dimensional structure of H-2Dd complexed with an immunodominant peptide from human immunodeficiency virus envelope glycoprotein 120. J Mol Biol 283, 179-191.
Ostergaard Pedersen, L., Nissen, M. H., Hansen, N. J., Nielsen, L. L., Lauenmoller, S. L., Blicher, T., Nansen, A., Sylvester-Hvid, C., Thromsen, A. R., and Buus, S. (2001). Efficient assembly of recombinant major histocompatibility complex class I molecules with preformed disulfide bonds. Eur J Immunol 31, 2986-2996.
Parks, C. A., Henning, K. R., Pavelko, K. D., Hansen, M. J., Van Keulen, V. P., Reed, B. K., Stone, J. D., Schrum, A. G., Gil, D., Kranz, D. M., et al. (2019). Breaking tolerance with engineered class I antigen-presenting molecules. Proc Natl Acad Sci USA 116, 3136-3145.
Rasmussen, M., Harndahl, M., Stryhn, A., Boucherma, R., Nielsen, L. L., Lemonnier, F. A., Nielsen, M., and Buus, S. (2014). Uncovering the peptide-binding specificities of HLA-C: a general strategy to determine the specificity of any MHC class I molecule. J Immunol 193, 4790-4802. Reynisson, B., Alvarez, B., Paul, S., Peters, B., and Nielsen, M. (2020). NetMHCpan-4.1 and NetMHCIIpan-4.0: improved predictions of MHC antigen presentation by concurrent motif deconvolution and integration of MS MHC eluted ligand data. Nucleic Acids Res 48, W449-W454.
Riedhammer, C., and Weissert, R. (2015). Antigen Presentation, Autoantigens, and Immune Regulation in Multiple Sclerosis and Other Autoimmune Diseases. Front Immunol 6, 322. Rossjohn, J., Gras, S., Miles, J. J., Turner, S. J., Godfrey, D. I., and Mccluskey, J. (2015). T cell antigen receptor recognition of antigen-presenting molecules. Annu Rev Immunol 33, 169-200. Saveanu, L., Carroll, O., Lindo, V., Del Val, M., Lopez, D., Lepelletier, Y., Greer, F., Schomburg, L., Fruci, D., Niedermann, G., et al. (2005). Concerted peptide trimming by human ERAP1 and ERAP2 aminopeptidase complexes in the endoplasmic reticulum. Nat Immunol 6, 689-697. Schneider, T. D., and Stephens, R. M. (1990). Sequence logos: a new way to display consensus sequences. Nucleic Acids Res 18, 6097-6100.
Sijts, E. J., and Kloetzel, P. M. (2011). The role of the proteasome in the generation of MHC class I ligands and immune responses. Cell Mol Life Sci 68, 1491-1502.
Smith, C., Gras, S., Brennan, R. M., Bird, N. L., Valkenburg, S. A., Twist, K. A., Burrows, J. M., Miles, J. J., Chambers, D., Bell, S., et al. (2014). Molecular imprint of exposure to naturally occurring genetic variants of human cytomegalovirus on the T cell repertoire. Sci Rep 4, 3993.
Thompson, C. B. (1995). New insights into V (D) J recombination and its role in the evolution of the immune system. Immunity 3, 531-539.
Vita, R., Mahajan, S., Overton, J. A., Dhanda, S. K., Martini, S., Cantrell, J. R., Wheeler, D. K., Sette, A., and Peters, B. (2019). The Immune Epitope Database (IEDB): 2018 update. Nucleic Acids Res 47, D339-D343.
Yewdell, J. W., and Bennink, J. R. (1999). Immunodominance in major histocompatibility complex class I-restricted T lymphocyte responses. Annu Rev Immunol 17, 51-88.
Zimmer, J., Andres, E., Donato, L., Hanau, D., Hentges, F., and de la Salle, H. (2005). Clinical and immunological aspects of HLA class I deficiency. QJM 98, 719-727.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A computer-assisted method for identifying or designing potential compounds to fit within or bind to an MHC chimera (“chimera”) or a functional portion thereof, or a computer-assisted method for identifying or designing a potential chimera or a functional portion thereof for binding to a desired compounds, or a computer-assisted method for identifying or designing a potential chimera of interest, optionally with regard to predicting area(s) of the chimera to be able to be manipulated, said method comprising using a computer system, optionally comprising one or more of a programmed computer comprising a processor, a data storage system, an input device, and an output device, and said method comprising steps comprising:
(a) inputting into the programmed computer through said input device data comprising the three-dimensional co-ordinates of a subset of the atoms from or pertaining to MHC chimera crystal structure, thereby generating a data set;
(b) comparing, using said processor, said data set to a computer database of structures stored in said computer data storage system, e.g., structures of compounds that bind or putatively bind or that are desired to bind to a chimera of the present invention or as to a chimera structure;
(c) selecting from said database, using computer methods, structure(s), optionally comprising structure(s) of chimera(s) that may bind to desired structures, and/or desired structures that may bind to certain chimera(s) or portions thereof, and/or portions of the chimera(s) that may be manipulated;
(d) constructing, using computer methods, a model of the selected structure(s); and
(e) outputting to said output device the selected structure(s); and
(f) optionally synthesizing one or more of the selected structure(s); and further
(g) optionally testing said synthesized selected structure(s) as or in a chimera.

2. The method of claim 1 comprising performing steps of the first embodiment for generation of chimera, or the second embodiment for generation of chimera, or third embodiment for generation of chimera.

3. The method of claim 1 comprising storage of data on a memory device, and the data including learning data set(s) for making comparisons and accepting or rejecting structures.

4. The method of claim 1 wherein step (f), or steps (f) and (g) are performed.

5. The method of claim 1 wherein steps (f) or (f) and (g) include synthesis and expression, said expression optionally being via a vector, or in a cell, a mammalian cell, or a human cell, or a non-human primate cell, or a non-human mammal cell, or a bacterial cell or in E. coli.

6. The method of claim 1 wherein steps (f) or (f) and (g) include incubating the chimera with a sample containing a peptide of interest (optionally binding of peptide to chimera promotes folding of the peptide/MHC/b2m protein complex).

7. The method of claim 6 further comprising detecting folding via antibody-based analysis (optionally comprising, ELISA, further optionally comprising contacting with antibody W6/32).

8. The method of claim 6 further comprising purification optionally comprising affinity-based purification, of pMHC proteins and elution of bound peptides (purified product).

9. The method of claim 8 further comprising analysis of purified product, optionally comprising proteomics analysis (optionally comprising performing mass spectrometry).

10. The method of claim 6 further comprising inputting data or results of performing steps of said claim(s) into the data set(s) or the memory device or stored data thereon for being employed in further computer implementation of a method of any preceding or following claim or any performance of any of the first embodiment for generation of chimera, or the second embodiment for generation of chimera, or third embodiment for generation of chimera.

11. The method of claim 1 further comprising; contacting T-cell(s) with the chimera to obtain modified T-cell(s) comprising T-cell(s) identified by recognition of a chimera peptide: MHC complex, and optionally further comprising expanding the T-cell(s) into a modified T-cell population, wherein the T-cell(s) used in the contacting can be isolated from a patient or subject, and optionally altered therefrom by having or introducing desired coding nucleic acid molecule(s) and/or by expressing desired product(s), optionally said introducing through a lentivirus system.

12. Use of modified T-cells for, or a method for, antigen targeting, said use or method comprising contacting modified T-cell(s) or the modified T-cell population of claim 11 with a sample.

13. The method of claim 11 or the use of claim 12 further comprising inputting data or results of performing steps of said claim(s) into the data set(s) or the memory device or stored data thereon for being employed in further computer implementation of a method of any preceding or following claim or any performance of any of the first embodiment for generation of chimera, or the second embodiment for generation of chimera, or third embodiment for generation of chimera.

14. The method or use of any of claims 1 to 13 further comprising genetically modifying a dendritic cell optionally comprising genetically modifying. a dendritic cell via a CRISPR system optionally comprising a CRISPR-Cas9 system, whereby coding for the chimeric is inserted into the genome of the dendritic cell, whereby there is a genetically modified dendritic cell that contains DNA coding for and/or expresses the chimera; and optionally expanding the modified dendritic into a modified T-cell population that contains DNA coding for and/or expresses the chimera, whereby the modified T-cell can target an antigen of interest.

15. The method of claim 14 wherein the antigen of interest is on a cell.

16. The method of claim 15 wherein the cell having the antigen of interest is a cancer cell, optionally a solid tumor cell or cell of a solid cancer.

17. Use of modified T-cells for, or a method for, antigen targeting, said use or method comprising contacting modified T-cell(s) or the modified T-cell population of claim 14, with a sample; optionally wherein the sample comprises a cell or a cancer cell or a solid tumor cell or a cell of a solid cancer.

18. The method of claim 14 or the use of claim 17 further comprising inputting data or results of performing steps of said claim(s) into the data set(s) or the memory device or stored data thereon for being employed in further computer implementation of a method of any preceding or claim or any performance of any of the first embodiment for generation of chimera, or the second embodiment for generation of chimera, or third embodiment for generation of chimera.

19. A composition, optionally a pharmaceutical or veterinary composition, comprising a chimera or a dendritic cell or a T-cell or a population of T-cells, any one of claims 11 or 14 to 16, and a diluent, carrier or excipient, optionally a pharmaceutically acceptable or veterinarily acceptable diluent, carrier or excipient.

20. A dendritic cell or a T-cell or a population of T-cells from any one of claims 11 or 14 to 16.


SEQUENCE LISTING (39 sequences)

SEQ ID NO: 1 - protein, 9 aa, synthetic construct
ELNRKMIYM

SEQ ID NO: 2 - protein, 9 aa, synthetic construct
AIFQSSMTK

SEQ ID NO: 3 - protein, 9 aa, synthetic construct
RYRPGTVAL

SEQ ID NO: 4 - protein, 9 aa, synthetic construct
FLRGRAYGL

SEQ ID NO: 5 - protein, 276 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTMHTHHAVSDHEATLRCWALSFYPAEITLTWQRDGEDQTQDTELVETRPAGDGTFQKWVAVVVPSGQEQRYTCHVQHEGLPKPLTLRWEP

SEQ ID NO: 6 - protein, 276 aa, synthetic construct
GSHSMRYFFTSVSRPGRGEPRFIAVGYVDDTQFVRFDSDAASORMEPRAPWIEQEGPEYWDGETRKVKAHSOTHRVDLGTLRGYYNQSEAGSHTVQIMYGCDVGSDWRFLRGYRQDAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLRWEP

SEQ ID NO: 7 - protein, 276 aa, synthetic construct
GSHSMRYFFTSVSRPGRGEPRFIAVGYVDDTQFVRFDSDAASORMEPRAPWIEQEGPEYWDQETRNVKAQSQTDRVDLGTLRGYYNQSEAGSHTIQIMYGCDVGSDWRFLRGYRQDAYDGKDYIALKEDLRSWTAADMAAQTTKHKWEAAHEAEQLRAYLEGTCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLRWEP

SEQ ID NO: 8 - protein, 276 aa, Homo sapiens
GSHSMRYFYTSVSRPGRGEPRFIAVGYVDDTQFVRFDSDAASORMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLRWEP

SEQ ID NO: 9 - protein, 276 aa, synthetic construct
GSHSMRYFDTSVSRPGRGEPRFIAVGYVDDTQFVRFDSDAASQRMEPRAPWIEQEGPEYWDRNTRIVKANSQTDRVDLGNLRGYYNQSEAGSHTVQSMYGCDVGSDWRFLRGYHQYAYDGKDYIALKEDRLSWTAADMAAQTTKHKWEAAHVAEQDRAYLEGTCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLRWEP

SEQ ID NO: 10 - protein, 276 aa, synthetic construct
GSHSMRYFDTSVSRPGRGEPRFISVGYVDDTQFVRFDSDAASQRMEPRAPWIEQEGPEYWDRNTRIFKTNSQTDRESLGNLRGYYNQSEAGSHTLQSMYGCDVGSDWRFLRGYNQYAYDGKDYIALKEDRLSWTAADMAAQITKHKWEAAHVAEQDRAYLEGTCVEWLRRYLENGKETLQRTDAPKTHMTHHAVSDHEATLRCWALSFYPAEITLTWQRDGTDQTQDTELVETRPAGDGTFQKWVAVVVPSGQEQRYTCHVQHEGLPKPLTLRWEP

SEQ ID NO: 11 - protein, 276 aa, Homo sapiens
GSHSMRYFDTAMSRPGRGEPRFISVGYVDDTQFVRFDSDAASPREEPRAPWIEQEGPEYWDRNTQIFKTNGQTDRESLRNLRGYYNQSEAGSHTLQSMYGCDVGPDGRLLRGHNQYAYDGKDYIALNEDRLSWTAADTAAQITQRKWEAARVAEQDRAYLEGTCVEWLRRYLENGKDTLERTDAPKTHMTHHAVSDHEATLRCWALSFYPAEITLTWQRDGTDQTQDTELVETRPAGDGTFQKWVAVVVPSGQEQRYTCHVQHEGLPKPLTLRWEP

SEQ ID NO: 12 - protein, 276 aa, synthetic construct
GSHSMRYFDTSVSRPGRGEPRFIAVGYVDDTQFVRFDSDAASQRMEPRAPWIEQEGPEYWDGETRKVKAQSQTHRVSLGTLRGYYNQSEAGSHTLQRMSGCDVGSDWRFLRGYDQSAYDGKDYIALKEDLRSWTAADMAAQTTKHKLEAAHVAEQLRAYLEGTCVEWLRRYLENGKETLQRTDAPKTMHTHHAVSDHEATLRCWALSFYPAEITLTWQRDGEDQTQDTELVETRPAGDGTFQKWVAVVVPSGQEQRYTCHVQHEGLPKPLTLRWEP

SEQ ID NO: 13 - protein, 275 aa, synthetic construct
GSHSMRYFDTSVSRPGRGEPRFISVGYVDDTQFVRFDSDAASQRMEPRAPWIEQEGPEYWDRETRKYKAQSQAHRVSLGNLRGYYKSEAGSHTLQRMSGCDVGSDWRFLRGYDQSAYDGKDYIALKEDLRSWTAADMAAQTTKHKLEAAHAAEQLRAYLEGTCVEWLRRYLENGKETLQRTDAPKTMHTHHAVSDHEATLRCWALSFYPAEITLTWQRDGEDQTQDTELVETRPAGDGTFQKWVAVVVPSGQEQRYTCHVQHEGLPKPLTLRWEP

SEQ ID NO: 14 - protein, 276 aa, Homo sapiens
GSHSMRYFDTAMSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRTDAPKTMHTHHAVSDHEATLRCWALSFYPAEITLTWQRDGEDQTQDTELVETRPAGDGTFQKWVAVVVPSGQEQRYTCHVQHEGLPKPLTLRWEP

SEQ ID NO: 15 - protein, 9 aa, synthetic construct
SLLMWITQC

SEQ ID NO: 16 - protein, 9 aa, synthetic construct
  MOD_RES 7: note = 3-amino-3-(2-nitrophenyl)-propionic acid)
FLRGRAXGL

SEQ ID NO: 17 - protein, 180 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQ

SEQ ID NO: 18 - protein, 180 aa, Homo sapiens
GSHSMRYFYTSVSRPGRGEPRFIAVGYVDDTQFVRFDSDAASORMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQ

SEQ ID NO: 19 - protein, 180 aa, synthetic construct
GSHSMRYFFTSVSRPGRGEPRFIAVGYVDDTQFVRFDSDAASORMEPRAPWIEQEGPEYWDGETRKVKAQSQTDRVDLGTLRGYYNQSEAGSHTIQIMYGCDVGSDWRFLRGYRQDAYDGKDYIALKEDLRSWTAADMAAQTTKHKWEAAHVAEQLRAYLEGTCVEWLRRYLENGKETLQ

SEQ ID NO: 20 - protein, 180 aa, Homo sapiens
GSHSMRYFFTSVSRPGRGEPRFIAVGYVDDTQFVRFDSDAASQRMEPRAPWIEQEGPEYWDGETRKVKAQSQTDRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQ

SEQ ID NO: 21 - protein, 180 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYRQDAYDGKDYIALKEDLRSWTAADMAAQTTKHKWEAAHVAEQLRAYLEGTCVEWLRRYLENGKETLQ

SEQ ID NO: 22 - protein, 180 aa, Homo sapiens
GSHSMRYFFTSVSRPGRGEPRFIAVGYVDDTQFVRFDSDAASORMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQ

SEQ ID NO: 23 - protein, 180 aa, Homo sapiens
GSHSMRYFFTSVSRPGRGEPRFIAVGYVDDTQFVRFDSDAASORMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLEGTCVEWLRRYLENGKETLQ

SEQ ID NO: 24 - protein, 180 aa, Homo sapiens
GSHSMRYFFTSVSRPGRGEPRFIAVGYVDDTQFVRFDSDAASORMEPRAPWIEQEGPEYWDQETRNVKAQSQTDRVDLGTLRGYYNQSEAGSHTIQIMYGCDVGSDGRFLRGYRQDAYDGKDYIALKEDLRSWTAADMAAQITKRKWEAAHEAEQLRAYLDGTCVEWLRRYLENGKETLQ

SEQ ID NO: 25 - protein, 9 aa, synthetic construct
LLFGYPVYV

SEQ ID NO: 26 - protein, 9 aa, Homo sapiens
SLLMWITQV

SEQ ID NO: 27 - protein, 9 aa, Homo sapiens
SLLMAITQV

SEQ ID NO: 28 - protein, 9 aa, Homo sapiens
RLRGVYAAL

SEQ ID NO: 29 - protein, 9 aa, Homo sapiens
QYNPIRTTF

SEQ ID NO: 30 - protein, 9 aa, Homo sapiens
QYNPIATTF

SEQ ID NO: 31 - protein, 9 aa, Human immunodeficiency virus 1
YPNVRIHNF

SEQ ID NO: 32 - protein, 9 aa, Homo sapiens
TEADVQQWL

SEQ ID NO: 33 - protein, 9 aa, synthetic construct
YPNVNIHNF

SEQ ID NO: 34 - protein, 276 aa, synthetic construct
GSHSMRYFFTAMSRPGRGEPRFISVGYVDDTQFVRFDSDAASPRMEPRAPWIEQEGPEYWDRETQKVKTHTQTHREDLRNLRGYYNQSEAGSHTLQRMYGCDVGPDGRLLRGHHQYAYDGKDYIALNEDLRSWTAADTAAQITQRKWEAARVAEQLRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 35 - protein, 276 aa, synthetic construct
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 36 - protein, 276 aa, synthetic construct
GSHSMRYFSTAMSRPGRGEPRFIAVGYVDDTQFVRFDSDAASPRMEPRAPWIEQEGPEYWDRETQKVKTHTQTDRENLRNARGYYNQSEAGSHILQMMFGCDLGPDGRLLRGHHQYAYDGKDYIALNEDLSSWTAADTAAQITQRKWEAARVAEQQRAYLEGLCVEGLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 37 - protein, 276 aa, synthetic construct
GSHSMRYFYTAMSRPGRGEPRFIAVGYVDDTQFVRFDSDAASPRTEPRAPWIEQEGPEYWDRNTQIFKTNTQTYRESLRNLRGYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 38 - protein, 9 aa, synthetic construct
AEFQSSMTK

SEQ ID NO: 39 - protein, 276 aa, synthetic construct
GSHSMRYFHTSVSRPGRGEPRFIAVGYVDDTQFVRFDSDAASQRKEPRAPWIEQEGPEYWDGETRKSKAQSQTDRVDLGTLRGYYNQSEAGSHTIQIMYGCDVGSDWRFLRGYRQDAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLRWEP